SEQUENCE LISTING (51 sequences)

SEQ ID NO: 1 - DNA, 354 bp, Artificial Sequence
  B7-VH Full Sequence
gaggtgcagctgttggagtctgggggaggcttggtacagcctggggggtccctgagactctcctgtgcagcctctggattcacctttagccactatgccatgaactgggtccgccaggctccagggaaggggctggagtgggtttcagccattaatagtaatggtgatagcacctactatccagacactgtgaaggaccgattcaccatctccagagacaattccaagaacacgctgtatctgcaaatgaacagcctgagagccgaggacacggccgtgtattactgtgcgagagatcggcgagcagggtactttgactactggggccagggcaccctggtcacagtctcctca

SEQ ID NO: 2 - protein, 118 aa, Artificial Sequence
  B7-VH  Full Sequence
EVQLLESGGGLVQPGGSLRLSCAASGFTFSHYAMNWVRQAPGKGLEWVSAINSNGDSTYYPDTVKDRFTISRDNSKNTLYLQMNSLRAEDTAVYYCARDRRAGYFDYWGQGTLVTVSS

SEQ ID NO: 3 - DNA, 75 bp, Artificial Sequence
  B7-VH FR1
gaggtgcagctgttggagtctgggggaggcttggtacagcctggggggtccctgagactctcctgtgcagcctct

SEQ ID NO: 4 - protein, 25 aa, Artificial Sequence
  B7-VH FR1
EVQLLESGGGLVQPGGSLRLSCAAS

SEQ ID NO: 5 - DNA, 24 bp, Artificial Sequence
  B7-VH CDRH1
ggattcacctttagccactatgcc

SEQ ID NO: 6 - protein, 8 aa, Artificial Sequence
  B7-VH CDRH1
GFTFSHYA

SEQ ID NO: 7 - DNA, 51 bp, Artificial Sequence
  B7-VH FR2
atgaactgggtccgccaggctccagggaaggggctggagtgggtttcagcc

SEQ ID NO: 8 - protein, 17 aa, Artificial Sequence
  B7-VH FR2
MNWVRQAPGKGLEWVSA

SEQ ID NO: 9 - DNA, 24 bp, Artificial Sequence
  B7-VH CDRH2
attaatagtaatggtgatagcacc

SEQ ID NO: 10 - protein, 8 aa, Artificial Sequence
  B7-VH CDRH2
INSNGDST

SEQ ID NO: 11 - DNA, 114 bp, Artificial Sequence
  B7-VH FR3
tactatccagacactgtgaaggaccgattcaccatctccagagacaattccaagaacacgctgtatctgcaaatgaacagcctgagagccgaggacacggccgtgtattactgt

SEQ ID NO: 12 - protein, 38 aa, Artificial Sequence
  B7-VH FR3
YYPDTVKDRFTISRDNSKNTLYLQMNSLRAEDTAVYYC

SEQ ID NO: 13 - DNA, 36 bp, Artificial Sequence
  B7-VH CDRH3
gcgagagatcggcgagcagggtactttgactactgg

SEQ ID NO: 14 - protein, 12 aa, Artificial Sequence
  B7-VH CDRH3
ARDRRAGYFDYW

SEQ ID NO: 15 - DNA, 30 bp, Artificial Sequence
  B7-VH J Region
ggccagggcaccctggtcacagtctcctca

SEQ ID NO: 16 - protein, 10 aa, Artificial Sequence
  B7-VH J Region
GQGTLVTVSS

SEQ ID NO: 17 - DNA, 330 bp, Artificial Sequence
  B7- VL (lambda) Full Sequence
gatatcgtgctgactcagccaccctcagcgtctgggacccccgggcagagggtcaccatctcctgctctggaagcagggacaacatcgggaagaattatgtgtcctggtaccagcagcttccaggaacagcccccaaactcctcatttataggaataatcagcggccctcaggggtccctgaccggttctctggctccaagtctggcacctcagcctccctggccatcagtgggctgcggtccgaggatgaggctgattattactgctcagcgtgggacaccagcctcaatgcttatgtcttcgggcctggcacccagctcaccgtcctc

SEQ ID NO: 18 - protein, 110 aa, Artificial Sequence
  B7- VL (lambda) Full Sequence
DIVLTQPPSASGTPGQRVTISCSGSRDNIGKNYVSWYQQLPGTAPKLLIYRNNQRPSGVPDRFSGSKSGTSASLAISGLRSEDEADYYCSAWDTSLNAYVFGPGTQLTVL

SEQ ID NO: 19 - DNA, 75 bp, Artificial Sequence
  B7- VL FR1
gatatcgtgctgactcagccaccctcagcgtctgggacccccgggcagagggtcaccatctcctgctctggaagc

SEQ ID NO: 20 - protein, 25 aa, Artificial Sequence
  B7- VL FR1
DIVLTQPPSASGTPGQRVTISCSGS

SEQ ID NO: 21 - DNA, 24 bp, Artificial Sequence
  B7- VL CDRL1
agggacaacatcgggaagaattat

SEQ ID NO: 22 - protein, 8 aa, Artificial Sequence
  B7- VL CDRL1
RDNIGKNY

SEQ ID NO: 23 - DNA, 51 bp, Artificial Sequence
  B7- VL FR2
gtgtcctggtaccagcagcttccaggaacagcccccaaactcctcatttat

SEQ ID NO: 24 - protein, 17 aa, Artificial Sequence
  B7- VL FR2
VSWYQQLPGTAPKLLIY

SEQ ID NO: 25 - DNA, 10 bp, Artificial Sequence
  B7- VL CDRL2
  misc_feature (10)..(10): n is null
aggaataatn

SEQ ID NO: 26 - protein, 4 aa, Artificial Sequence
  B7- VL CDRL2
  MISC_FEATURE (4)..(4): Xaa is null
RNNX

SEQ ID NO: 27 - DNA, 108 bp, Artificial Sequence
  B7- VL FR3
cagcggccctcaggggtccctgaccggttctctggctccaagtctggcacctcagcctccctggccatcagtgggctgcggtccgaggatgaggctgattattactgc

SEQ ID NO: 28 - protein, 36 aa, Artificial Sequence
  B7- VL FR3
QRPSGVPDRFSGSKSGTSASLAISGLRSEDEADYYC

SEQ ID NO: 29 - DNA, 27 bp, Artificial Sequence
  B7- VL CDRL3
tcagcgtgggacaccagcctcaatgct

SEQ ID NO: 30 - protein, 9 aa, Artificial Sequence
  B7- VL CDRL3
SAWDTSLNA

SEQ ID NO: 31 - DNA, 36 bp, Artificial Sequence
  B7- VL J Region
tatgtcttcgggcctggcacccagctcaccgtcctc

SEQ ID NO: 32 - protein, 12 aa, Artificial Sequence
  B7- VL J Region
YVFGPGTQLTVL

SEQ ID NO: 33 - protein, 17 aa, Artificial Sequence
  PEPTIDE #49
CTSAQKASQVAEEAADC

SEQ ID NO: 34 - protein, 17 aa, Artificial Sequence
  PEPTIDE #49-SC
CSSEVQAAKADTEAQAC

SEQ ID NO: 35 - protein, 17 aa, Artificial Sequence
  PEPTIDE #50
CKASQVAEEAADAAQEC

SEQ ID NO: 36 - protein, 17 aa, Artificial Sequence
  PEPTIDE #50-SC
CDEAVAQAAAEKEAQSC

SEQ ID NO: 37 - protein, 21 aa, Artificial Sequence
  PEPTIDE #49+50
CTSAQKASQVAEEAADAAQEC

SEQ ID NO: 38 - protein, 17 aa, Artificial Sequence
  PEPTIDE #78
CTDLINRMGQAARLAGC

SEQ ID NO: 39 - protein, 18 aa, Artificial Sequence
  EPITOPE OF B7
TSAQKASQVAEEAADAAQ

SEQ ID NO: 40 - protein, 321 aa, Escherichia coli
  MISC_FEATURE: EspB, EPEC Accession number WP_001091991.1
MNTIDNNNAAIAVNSVLSSTTDSTSSTTTSTSSISSSLLTDGRVDISKLLLEVQKLLREMVTTLQDYLQKQLAQSYDIQKAVFESQNKAIDEKKAGATAALIGGAISSVLGILGSFAAINSATKGASDVAQQAASTSAKSIGTVSEASTKALAKASEGIADAADDAAGAMQQTIATAAKAASRTSGITDDVATSAQKASQVAEEAADAAQELAQKAGLLSRFTAAAGRISGSTPFIVVTSLAEGTKTLPTTISESVKSNHDINEQRAKSVENLQASNLDTYKQDVRRAQDDISSRLRDMTTTARDLTDLINRMGQAARLAG

SEQ ID NO: 41 - DNA, 945 bp, Escherichia coli
  misc_feature: EspB, EPEC Accession number AAB69980.1
atgaatactattgattatactaatcaagtaatgacggttaattctgtttcggagaatactaccggctctaatgcaattaccgcatctgctattaattcatctttgcttaccgatggtaaggtcgatgtttctaaactgatgctggaaattcaaaaactcctgggcaagatggtgcgtatattgcaggattaccaacagcaacagttgtcgcagagctatcagatccaactggccgtttttgagagccagaataaagccattgatgaaaaaaaggccgctgcaacagccgctctggttggtggggctatttcatcagtattggggatcttaggctcttttgcagcaattaacagtgctacgaaaggcgcgagtgatattgctcaaaaaaccgcctctacatcttctaaggctattgatgcggcttctgatactgcgactaaaacgttgactaaggcaacggaaagcgttgctgatgctgttgaagatgcatccagcgtgatgcagcaagcgatgactacagcaacgagagcggccagccgtacatccgacgttgctgatgacattgccgattctgctcagagagcttctcagctggctgaaaacgctgcagatgccgctcagaaggcaagtcgggcaagccgctttatggctgcagtagataagattactggctctacaccatttattgccgttaccagtcttgccgaaggcacgaagacattgccaacaacggtatctgaatcagtcaaatctaaccatgagattagcgaacagcgttataagtctgtggagaacttccagcagggtaatttggatctgtataagcaagaagttcgcagagcgcaggatgatatcgctagccgtctgcgtgatatgacaacagccgctcgcgatctcactgatcttcagaatcgtatgggtcaatcggttcgcttagctgggtaa

SEQ ID NO: 42 - protein, 380 aa, Escherichia coli
  MISC_FEATURE: EspD, EPEC Accession number WP_000935767.1
MLNVNSDIQSMRSGASAATATSGINQPEVTSALDLQLVKSTAPSASWTESTALPTPPAGHSLVTPSAAEDVLSKLFGGISGEVRSRTEGTEPQRSTQNASSGYPYLSQVNNVDPQAMMMMVTLLSLDASAQKVASMKNSNEIYTDGQNKALDNKTLEFKKQLEEQQKAEEKAQKSKIVGQVFGWLGVAVTAIAAIFNPALWAVVAVSATAMALQTAVDVMGDKAPQALKTAAQVFGGVSIAASILTAGIGGVSSLMSKVGDVANKVGSNIVKTVTKLADVFVENVVSKVAATANGFTTSARSIGTTVLNNDSAQYSILAQLSAYAVQNLTRQSENLGESAKVELDKAAAELRNQASYLQNASQLISDSARVNSRIVSGRV

SEQ ID NO: 43 - DNA, 1000 bp, Escherichia coli
  misc_feature: EspD, EPEC Accession number CAI43861.1
atgctcaatgtaaatagcgatatccagtcgatgaggtctggggccagcgctgctacggctacttcaggtattaatcagcctgaagtgacttccgcgctggatttacaactggttaaatccacggccccatcagcaagttggacagaaagtaccgctttaccaacgccaccggcaggccactcattagtgacgccctctgctgctgaggatgtcctgagtaaactgtttggtggtattagtggtgaagttaggagtcgaacagagggaaccgaaccacagcgcagcactcagaatgcaagctctggctatccttatctttctcaggtgaataacgttgaccctcaggccatgatgatgatggtcaccctgttatccctggatgcttcagcgcagaaagttgcgagtatgaaaaactccaacgagatctacacggatgggcaaaataaggcgctggataataaaacgctggagtttaaaaagcagctcgaagaacaacagaaagccgaagagaaagcacaaaaaagtaaaattgttggtcaggtctttggttggttgggcgtcgcggtaactgctattgcagctatttttaacccggcactctgggctgttgttgccgttagcgcaacagcaatggcactgcaaactgcagttgatgtaatgggcgataaggccccgcaggcgttaaagacagcagcgcaagtctttggcggagtatctattgctgcaagtattctgacagccggcattggcggggtgtcttcacttatgtctaaagttggcgatgtcgctaacaaagttggttcaaacatcgtgaaaacagttactaaactggccgatgtatttgttgagaacgttgtttcaaaagttgcggcaacggcaaatggatttacgacctctgcacgttctattgggacaactgtgctgaataatgattctgcgcaatacagcatattggctcagttatctgcttacgcggtgcaaaacttaactcgacagagtg

SEQ ID NO: 44 - protein, 21 aa, Artificial Sequence
  PEPTIDE 49-50 SC
CAEATQEADVAEQASKASQAC

SEQ ID NO: 45 - protein, 312 aa, Escherichia coli
  MISC_FEATURE: EHEC-EspB
MNTIDNTQVTMVNSASESTTGASSAVAASALSIDSSLLTDGKVDICKLMLEIQKLLGKMVTLLQDYQQKQLAQSYQIQQAVFESQNKAIEEKKAAATAALVGGIISSALGILGSFAAMNNAAKGAGEIAEKASSASSKAAGAASEVANKALVKATESVADVAEEASSAMQKAMATTTKAASRASGVADDVAKASDFAEDLADAAEKTSRINKLLNSVDKLTNTTAFVAVTSLAEGTKTLPTTISESVKSTHEVNEQRAKSLENFQQGNLELYKQDVRRTQDDITTRLRDITSAVRDLLEVQNRMGQSGRLAG

SEQ ID NO: 46 - protein, 321 aa, Citrobacter rodentium
  MISC_FEATURE: C. rodentium (CR)-EspB
MNTIDYNNAAIAVNPVVSSTTDSASSTTTLTSSSSSSLLTAGKVDISKLLLEVQKLMREMVTTLQDYLQRQLAQSYNIQQAIFQSQNKAIEEKKAGATAALIGGAISSVLGILGSFAAINSATKGASDVVQKATSASAKSVNTASEVSTKALVKASESIADAADETVGTLQQGIAAATKAANRTSGIADDVTTSAQKASQVAEEAADAAQDLTRKANLLSRFTAAAGRISGSTAFVVVTSLAEGTKTLPTTISESVKSNHDINEQRAKSVENLQASNLETYKQDVRRSQDDIASRLRDMTATARDLNDLINRMGQSARLAG

SEQ ID NO: 47 - protein, 409 aa, Salmonella
  MISC_FEATURE: Salmonella-EspB A
MLISNVGINPAAYLNNHSVENSSQTASQSVSAKDILNSIGISSSKVSDLGLSPTLSAPAPGVLTQTPGTITSFLKASIQNTDMNQDLNALANNVTTKANEVVQTQLREQQAEVGKFFDISGMSSSAVALLAAANTLMLTLNQADSKLSGKLSLVSFDAAKTTASSMMREGMNALSGSISQSALQLGITGVGAKLEYKGLQNERGALKHNAAKIDKLTTESHSIKNVLNGQNSVKLGAEGVDSLKSLNMKKTGTDATKNLNDATLKSNAGTSATESLGIKDSNKQISPEHQAILSKRLESVESDIRLEQNTMDMTRIDARKMQMTGDLIMKNSVTVGGIAGASGQYAATQERSEQQISQVNNRVASTASDEARESSRKSTSLIQEMLKTMESINQSKASALAAIAGNIRA

SEQ ID NO: 48 - protein, 27 aa, Artificial Sequence
  peptide #9-12
SISSSLLTDGRVDISKLLLEVQKLLRE

SEQ ID NO: 49 - protein, 11 aa, Artificial Sequence
  Epitope peptide
KASQVAEEAAD

SEQ ID NO: 50 - protein, 7 aa, Unknown
  Cyanobacteria
  MISC_FEATURE: Microcystin general sequence
  MISC_FEATURE (1)..(1): Xaa is equal to d-Ala
  MISC_FEATURE (2)..(2): Xaa is equal to any amino acid
  MISC_FEATURE (3)..(3): Xaa is equal to is d-erythro-beta-methylaspartic acid
  MISC_FEATURE (4)..(4): Xaa is equal to any amino acid
  MISC_FEATURE (5)..(5): Xaa is equal to (all-S,all-E)-3-amino-9-methoxy-2,6,8-trimethyl-10- phenyldeca-4,6 -dienoic acid
  MISC_FEATURE (6)..(6): Xaa is equal to d-Glu
  MISC_FEATURE (7)..(7): Xaa is equal to N-methyldehydroalanine
XXXXXXX

SEQ ID NO: 51 - protein, 7 aa, Unknown
  Cyanobacteria
  MISC_FEATURE: Microcystin LR
  MISC_FEATURE (1)..(1): Xaa is equal to d-Ala
  MISC_FEATURE (3)..(3): Xaa is equal to d-erythro-beta-methylaspartic acid
  MISC_FEATURE (5)..(5): Xaa is equal to (all-S,all-E)-3-amino-9-methoxy-2,6,8-trimethyl-10-phenyldeca-4,6 -dienoic acid
  MISC_FEATURE (6)..(6): Xaa is equal to d-Glu
  MISC_FEATURE (7)..(7): Xaa is equal to N-methyldehydroalanine
XLXRXXX

(19) United States
(12) Patent Application Publication
(10) Pub. No.: US 2025/0264433 A1
(43) Pub. Date: Aug. 21, 2025

(54) AN INTEGRATED MULTI-METHOD ELECTROCHEMICAL BIOSENSOR FOR RAPID-ON-SITE DETECTION AND/OR QUANTIFICATION OF SMALL MOLECULE TARGETS IN A SAMPLE

(71) Applicant: THE STATE OF ISRAEL, MINISTRY OF AGRICULTURE & RURAL DEVELOPMENT, AGRICULTURAL, Rishon Lezion (IL)

(72) Inventors: Sefi VERNICK, Rishon Lezion (IL); Abraham Oluwafemi OGUNGBILE, Rishon Lezion (IL); Orr h. SHAPIRO, Rishon Lezion (IL)

(21) Appl. No.: 18/254,458

(22) PCT Filed: Nov. 25, 2021
(86) PCT No.: PCT/IL21/51409

§ 371 Date: May 25, 2023

Related U.S. Application Data
(60) Provisional application No. 63/118,371, filed on Nov. 25, 2020.

(51) Int. Cl. G01N 27/327; G01N 27/02; G01N 27/48; G01N 33/487; G01N 33/543; G01N 33/58

(52) U.S. Cl. CPC G01N 27/3276; G01N 27/026; G01N 27/48; G01N 33/48707; G01N 33/5438; G01N 33/581

(57) ABSTRACT

The invention relates to immunological-based biosensor chip systems, devices, kits and diagnostic methods for detection, quantification and/or monitoring of at least one target compound, specifically, at least one small molecule compound, specifically, cyanotoxins in a sample. The present disclosure relates to a biosensor chip system usable for identifying and/or quantifying and/or monitoring at least one target in a sample. More specifically, the system comprises at least one of: at least one first and at least one second chip devices. It should be noted that the first chip device comprises a first plurality of electrodes connectable to at least one electronic device. It should be further noted that the first plurality of electrodes is configured for electrochemical impedance spectroscopy (EIS) analysis of the sample. The second chip device comprises a second plurality of electrodes connectable to at least one electronic device. The second plurality of electrodes is configured for electrochemical voltammetry or amperometry analysis of said sample.

The Sequence Listing in ASCII text file format of 33,087 bytes in size, created on Nov. 29, 2023, with the file name “2023-11-29Sequence Listing_VERNICK1,” filed in the U.S. Patent and Trademark Office on even date herewith, is hereby incorporated herein by reference.

### FIELD OF THE INVENTION

The present invention generally relates to diagnostic systems, devices, kits, methods and uses thereof in detection of target molecules. More specifically, the invention relates to immunological-based biosensor chip, devices, kits and diagnostic methods for detection, quantification and/or monitoring of at least one target compound, specifically, at least one small molecule compound, specifically, cyanotoxins in a sample.

### BACKGROUND ART

References considered to be relevant as background to the presently disclosed subject matter are listed below:
[1] T. D. Bucheli, Phytotoxins: environmental micropollutants of concern? Environ. Sci. Technol. 48 (2014) 13027-13033, https://doi.org/10.1021/es504342w.
[2] M. L. Saker, J. Fastner, E. Dittmann, G. Christiansen, V. M. Vasconcelos, Variation between strains of the cyanobacterium Microcystis aeruginosa isolated from a Portuguese river, J. Appl. Microbiol. 99 (2005) 749-757, https://doi.org/10.1111/j.1365-2672.2005.02687.x.
[3] C. Flores, J. Caixach, An integrated strategy for rapid and accurate determination of free and cell-bound microcystins and related peptides in natural blooms by liquid chromatography-electrospray-high resolution mass spectrometry and matrix-assisted laser desorption/ionization, J. Chromatogr. A 1407 (2015) 76-89, https://doi.org/10.1016/j.chroma.2015.06.022.
[4] S. Bogialli, C. Bortolini, I. M. Di Gangi, F. N. Di Gregorio, L. Lucentini, G. Favaro, P. Pastore, Liquid chromatography-high resolution mass spectrometric methods for the surveillance monitoring of cyanotoxins in freshwaters, Talanta 170 (2017) 322-330, https://doi.org/10.1016/j.talanta.2017.04.033.
[5] M. G. Antoniou, A. A. de la Cruz, D. D. Dionysiou, Cyanotoxins: new generation of water contaminants, J. Environ. Eng. 131 (2005) 1239-1243, https://doi.org/10.1061/(ASCE)0733-9372(2005)131:9(1239).
[6] R. Wood, Acute animal and human poisonings from cyanotoxin exposure—a review of the literature, Environ. Int. 91 (2016) 276-282, https://doi.org/10.1016/j.envint.2016.02.026.
[7] United Nations, Back to our Common Future: Sustainable Development in the 21^{st} Century (SD21) project, Back to Our Common Futur. Sustain. Dev. 21st Century Proj. (2012) 39.
[8] J. Kulys, U. Bilitewski, R. D. Schmid, The kinetics of simultaneous conversion of hydrogen peroxide and aromatic compounds at peroxidase electrodes, J. Electroanal. Chem. 321 (1991) 277-286, (91)85601-K.
[9] K. K. Schrader, M. Q. De Regt, P. D. Tidwell, C. S. Tucker, S. O. Duke, Compounds with selective toxicity towards the off-flavor metabolite-producing cyanobacterium Oscillatoria cf. chalybea, Aquaculture 163 (1998) 85-99.
[10] C. S. Tucker, Off-flavor problems in aquaculture, Rev. Fish. Sci. 8 (2000) 45-88, https://doi.org/10.1080/10641260091129170.
[11] United Nations, The United Nations world water development report 2015: water for a sustainable world—UNESCO Digital Library, 2015. [symbol] https://unesdoc.unesco.
[12] W. W. Carmichael, Cyanotoxins secondary metabolites—the cyanotoxins, J. Appl. Bacteriol. 72 (1992) 445-459.
[13] M. G. Weller, Immunoassays and biosensors for the detection of cyanobacterial Toxins in water, Sensors 13 (2013) 15085-15112, https://doi.org/10.3390/s131115085.
[14] E. M. L. Janssen, Cyanobacterial peptides beyond microcystins—a review on cooccurrence, toxicity, and challenges for risk assessment, Water Res. 151 (2019) 488-499, https://doi.org/10.1016/j.watres.2018.12.048.
[15] M. Y. Cheung, S. Liang, J. Lee, Toxin-producing cyanobacteria in freshwater: a review of the problems, impact on drinking water safety, and efforts for protecting public health, J. Microbiol. 51 (2013) 1-10, https://doi.org/10.1007/s12275-013-2549-3.
[16] S. B. Watson, J. Ridal, G. L. Boyer, Taste and odour and cyanobacterial toxins: impairment, prediction, and management in the Great Lakes, Can. J. Fish. Aquat. Sci. 65 (2008) 1779-1796, https://doi.org/10.1139/F08-084.
[17] B. G. Kotak, R. W. Zurawell, Cyanobacterial toxins in Canadian freshwaters: a review, Lake Reserv. Manag. 23 (2007) 109-122, https://doi.org/10.1080/07438140709353915.
[18] S. D. Richardson, Water analysis: emerging contaminants and current issues, Anal. Chem. 81 (2009) 4645-4677, https://doi.org/10.1021/ac9008012.
[19] S. D. Richardson, T. A. Ternes, Water analysis: emerging contaminants and current issues, Anal. Chem. 83 (2011) 4616-4648, https://doi.org/10.1021/ac200915r.
[20] P. C. Turner, A. J. Gammie, K. Hollinrake, G. A. Codd, Pneumonia associated with contact with cyanobacteria, Br. Med. J. 300 (1990) 1440-1441, https://doi.org/10.1136/bmj.300.6737.1440.
[21] W. Jochimsen, E. M. Carmichael, W. W. An, J. Cardo, D. M. Cookson, S. T. Holmes, C. E. M. Antunes, B. C. Melo Filho, D. A. Lyra, T. M. Barreto, V. S. T. Azevedo, S. M. F. O, Jarvis, Liver failure and death after exposure to microcystins, N. Engl. J. Med. 338 (1998) 873-878, https://doi.org/10.1080/13504509.2013.856048. M4—Citavi.
[22] G. Codd, S. Bell, K. Kaya, C. Ward, K. Beattie, J. Metcalf, Cyanobacterial toxins, exposure routes and human health, Eur. J. Phycol. 34 (1999) 405-415, https://doi.org/10.1080/09670269910001736462.
[23] W. W. Carmichael, The toxins of cyanobacteria, Sci. Am. 270 (1994) 78-86,https://doi.org/10.1038/scientificamerican0194-78.
[24] W. W. Carmichael, S. M. F. O. Azevedo, J. S. An, R. J. R. Molica, E. M. Jochimsen, S. Lau, K. L. Rinehart, G. R. Shaw, G. K. Eaglesham, Human fatalities form cyanobacteria: chemical and biological evidence for cyanotoxins, Environ. Health Perspect. 109 (2001) 663-668, https://doi.org/10.1289/ehp.01109663.
[25] Z. Svir ̌ cev, D. Drobac, N. Tokodi, B. Mijovi\acute{ } c, G. A. Codd, J. Meriluoto, Toxicology of microcystins with reference to cases of human intoxications and epidemiological investigations of exposures to cyanobacteria and cyanotoxins, Arch. Toxicol. 91 (2017) 621-650, https://doi.org/10.1007/s00204-016-1921-6.
[26] G. A. Codd, Cyanobacterial toxins, the perception of water quality, and the prioritisation of eutrophication control, Ecol. Eng. 16 (2000) 51-60, https://doi.org/10.1016/S0925-8574(00)00089-6.
[27] WHO, Guidelines for Drinking-water Quality Second Addendum to Third Edition WHO Library Cataloguing-in-Publication Data (2008) 17-19. [symbol] https://101. org/10.1016/S1462-0758(00)00006-6 [symbol] .
[28] G. A. Codd, L. F. Morrison, J. S. Metcalf, Cyanobacterial toxins: risk management for health protection, Toxicol. Appl. Pharmacol. 203 (2005) 264-272, https://doi.org/10.1016/j.taap.2004.02.016.
[29] U. States, E. P. Agency, Efit4 (2014) 1-11. [30] U. P. App, Freshwater HABs Newsletter (2017).
[31] I. Sanseverino, D. C. Ant\acute{ } onio, Cyanotoxins: methods and approaches for their analysis and detection, 2017. [symbol] https://doi.org/10.2760/36186.
[32] J. Rapala, K. Erkomaa, J. Kukkonen, K. Sivonen, K. Lahti, Detection of microcystins with protein phosphatase inhibition assay, high-performance liquid chromatography-UV detection and enzyme-linked immunosorbent assay: comparison of methods, Anal. Chim. Acta 466 (2002) 213-231, https://doi.org/10.1016/50003-2670(02)00588-3.
[33] T. A. M. Msagati, B. A. Siame, D. D. Shushu, Evaluation of methods for the isolation, detection and quantification of cyanobacterial hepatotoxins, Aquat. Toxicol. 78 (2006) 382-397, https://doi.org/10.1016/j.aquatox.2006.03.011.
[34] M. E. Silva-Stenico, R. C. Neto, I. R. Alves, L. A. B. Moraes, T. K. Shishidoa, M. F. Fiore, Hepatotoxin microcystin-LR extraction optimization, J. Braz. Chem. Soc. 20 (2009) 535-542, https://doi.org/10.1590/S0103-50532009000300019.
[35] J. Jehli ̌ cka, H. G. M. Edwards, A. Oren, Raman spectroscopy of microbial pigments, Appl. Environ. Microbiol. 80 (2014) 3286-3295, https://doi.org/10.1128/AEM.00699-14.
[36] H. kholafazad Kordasht, S. Hassanpour, B. Baradaran, R. Nosrati, M. Hashemzaei, A. Mokhtarzadeh, M. de la Guardia, Biosensing of microcystins in water samples; recent advances, Biosens. Bioelectron. 165 (2020), 112403, https://doi.org/10.1016/j.bios.2020.112403.
[37] I. Y. Massey, P. Wu, J. Wei, J. Luo, P. Ding, H. Wei, F. Yang, A mini-review on detection methods of microcystins, Toxins 12 (2020) 1-32, https://doi.org/10.3390/toxins12100641.
[38]V. Vogiazi, A. De La Cruz, S. Mishra, V. Shanov, W. R. Heineman, D. D. Dionysiou, A comprehensive review: development of electrochemical biosensors for detection of cyanotoxins in freshwater, ACS Sens. 4 (2019) 1151-1173, https://doi.org/10.1021/acssensors.9b00376.
[39] L. Hou, Y. Ding, L. Zhang, Y. Guo, M. Li, Z. Chen, X. Wu, An ultrasensitive competitive immunosensor for impedimetric detection of microcystin-LR via antibody-conjugated enzymatic biocatalytic precipitation, Sens. Actuators B Chem. 233 (2016) 63-70, https://doi.org/10.1016/j.snb.2016.04.034.
[40] M. Boffadossi, A. Di Tocco, G. Lassabe, M. Pirez-Schirmer, S. N. Robledo, H. Fern\acute{ } andez, M. A. Zon, G. Gonz\acute{ } alez-Sapienza, F. J. Ar\acute{ } evalo, Development of an impedimetric immunosensor to determine microcystin-LR. New approaches in the use of the electrochemical impedance spectroscopy was used in determining to determine kinetic parameters of immunoreactions, Electrochim. Acta 353 (2020), 136621, https://doi.org/10.1016/j.electacta.2020.136621.
[41] X. Sun, L. Guan, H. Shi, J. Ji, Y. Zhang, Z. Li, Determination of microcystin-LR with a glassy carbon impedimetric immunoelectrode modified with an ionic liquid and multiwalled carbon nanotubes, Microchim. Acta 180 (2013) 75-83, https://doi.org/10.1007/s00604-012-0912-4.
[42] I. I. Suni, Impedance methods for electrochemical sensors using nanomaterials, TrAC Trends Anal. Chem. 27 (2008) 604-611, https://doi.org/10.1016/j.trac.2008.03.012.

### BACKGROUND OF THE INVENTION

Cyanobacteria, commonly referred to as green-blue algae, are a ubiquitous group of photosynthetic bacteria present in freshwater and marine environments, as well as in many habitats across the globe [1-4]. The ability of certain cyanobacterial species to proliferate in environments with high nutrient loads and light intensities results in the rapid development of harmful cyanobacterial blooms (HCBs), especially in eutrophic water bodies[5,6]. Eutrophication and global climate change are driving the proliferation and expansion of HCBs [7]. These HCBs are recognized as a major threat to the management of open freshwater bodies for aquaculture, drinking, recreation, and tourism worldwide[8-10], and it is projected that by 2050 the incidence of HCBs-contaminated lakes will increase by at least 20%[11]. During blooming events, these HCBs can contain multiple cyanotoxin (CT)-producing species [12-14]. Following cell rupture, a mass of toxins is released into the water, leading to public health and environmental issues [15-17] associated with increased animal and human poisonings[18,19].

Among the toxins produced by different cyanobacterial strains during HCBs, Microcystins (MCs) are of particular concern[20,21]. MCs are cyclic heptapeptides produced by many genera, including Microcystis, Anabaena, Aphanizomenon, and Planktothrix[22]. Microcystin-LR (MC-LR), the most common and studied variant[2,14], accounts for most reported poisonings. MC-LR is a hepatotoxin, a carcinogen, and a potent inhibitor of two key enzymes in cellular processes, protein phosphatases 1A (PP1A) and 2A (PP2A)[23-26]. Due to the toxicity and risks associated with MCs, a provisional guideline for total MC-LR in drinking water of 1 μg/L was established by the World Health Organization (WHO) in 1998[27] and has been used by many countries to set their health alert or drinking water regulations [5,28-31]. Subsequently, it became clear that a fast, reliable, and sensitive detection method for monitoring low MC-LR levels was needed to manage the drinking water and recreational health risks. Methods that have been developed over the years include bioassays, immunoassays, and several chromatographic techniques [32-34]. Although these methods provide sensitive detection and accurate identification, the operating costs and the need for skilled personnel and dedicated laboratory limit their application in the operation of water bodies[13,35]. Moreover, the hazards associated with the toxins produced during blooming events are highly time-dependent. Conventional detection methods generally rely on occasional sampling, and heavy toxin peaks, which require very short sampling intervals, could be completely missed[13]. Therefore, an affordable, portable, and highly sensitive tool that can overcome the limitations of current methods and allow on-site, real-time monitoring is urgently needed.

Recently, the development of electrochemical (EC) biosensors for the detection of environmental micro-pollutants has received much attention due to their high specificity, ultra-sensitivity, and broad dynamic range[36,37]. In particular, EC immunosensors contain a biorecognition element in the form of an antibody that reacts specifically with an analyte. This biological binding event occurs at the interface between the bulk solution and the surface of an electrode. The electrochemical cell transduces the immunoreaction into a measurable electrical signal[38]. Immunosensors employ chemically and biochemically modified electrodes to implement different measurement techniques such as amperometry, potentiometry, voltammetry, or electrochemical impedance spectroscopy (EIS). EIS-based immunosensors are particularly suited for on-site applications since they are label-free and require little to no sample preparation.

The study and development of EIS-based immunosensors for MC-LR detection have been pursued recently [39-41]. The transduction mechanism generally relies on the binding of a target analyte to an antibody-functionalized chip (biochip)[38,42]. In faradic EIS, such binding affects the kinetics of electron transfer between a redox probe and the electrode surface, resulting in changes to the impedance spectra and specifically, the charge transfer resistance (R_{ct}) component[39]. The combination of selectivity, provided by specific antibodies, and sensitivity, which is intrinsic to EC transduction, makes impedimetric immunosensors excellent candidates for user-friendly and inexpensive on-site diagnostic devices, and the development of such devices for MC-LR detection in water reservoirs is highly desirable.

Thus, there is an urgent need for an advanced portable detection tool to enable a frequent examination and quick monitoring of MCs in fishponds, drinking water reservoirs, and other surface water. This would help mitigate the risks associated with the safety of aquaculture produce as well as drinking water, and rapidly apply the necessary remedial measures.

### SUMMARY OF THE INVENTION

A first aspect of the present disclosure relates to a biosensor chip system usable for identifying and/or quantifying and/or monitoring at least one target in a sample. More specifically, the system comprises at least one of: at least one first and at least one second chip devices. It should be noted that the first chip device comprises a first plurality of electrodes connectable to at least one electronic device. The at least one of the electrodes is a working electrode. More specifically, in some embodiments, the working electrode is connected directly or indirectly to at least one target binding site and/or moiety. Still further, in some embodiments, the target binding site and/or moiety specifically binds the at least one target or any component thereof. It should be further noted that the first plurality of electrodes is configured for electrochemical impedance spectroscopy (EIS) analysis of the sample. In some alternative and/or additional embodiments the system of the present disclosure may comprise in addition to the first device, or as an alternative to the first device, at least one second chip device that comprises: a second plurality of electrodes connectable to at least one electronic device. It should be noted that the at least one of the electrodes is a working electrode. The at least working electrode of the second device is connected directly or indirectly to the target or any component thereof. It should be noted that this second plurality of electrodes is configured for electrochemical voltammetry or amperometry analysis of the sample.

In some embodiments, the biosensor chip system of the present disclosure further comprises a packaging assembly configured to sealably enclose the electrodes portion of the substrate and define at least one measurement chamber encompassing the electrodes.

In some embodiments, the system disclosed herein comprises at least one of the first device. In yet some alternative embodiments, the system of the present disclosure may comprise at least one second device. In yet some alternative embodiments, the disclosed systems may comprise at least one first device and at least one second device.

Thus, in yet some further embodiments, the biosensor chip system of the present disclosure, comprises the at least one first and the at least one second chip devices. According to such embodiments, the respective pluralities of electrodes of the first and second chip devices are positioned in respective first and second separated measurement chambers.

Still further, in some embodiments, the biosensor chip system of the present disclosure may further comprise at least one inlet for introducing the sample into the measurement chamber; and at least one inlet filter for selectively passing the sample from the inlet into the measurement chamber.

In some embodiments, the biosensor chip system of the present disclosure comprises an outlet formed in the packaging assembly and at least one outlet filter for selectively passing sample material from the measurement chamber to the outlet.

In certain embodiments of the biosensor chip system of the preset disclosure, the packaging assembly comprises a base portion configured to receive the electrodes portion of the substrate, and a cover portion having an open cavity and configured to sealably attach to the base portion over the electrodes portion of the substrate and define the measurement chamber by its open cavity.

In some embodiments of the biosensor chip system of the present disclosure, the first and second plurality of electrodes of at least one of the first and second chip devices comprises at least one working electrode, at least one counter electrode configured to vary electrical potential and enable current transmission into the measurement chamber, and at least one reference electrode for measuring electrical voltage between the at least one working electrode and the at least one reference electrode.

Still further, in some embodiments of the biosensor chip system, the at least one electronic device comprises one or more potentiostat circuitries connected at the one of first and second chip devices.

In some further embodiments, of the biosensor chip system according to the invention, the at least one electronic device comprises a plurality of potentiostat circuitries. More specifically, the system comprising a plurality of measurement chambers comprising at least one first measurement chamber associated with the first chip device and/or at least one second measurement chamber associated with the second chip device. Each of the measurement chambers comprises at least three of the plurality of electrodes defining a working electrode, a reference electrode, and a counter electrode, and is associated with respective potentiostat circuitries electrically connected to the at least three electrodes of its respective measurement chamber.

In some embodiments of the biosensor chip system of the present disclosure, the plurality of potentiostat circuitries comprises at least one first potentiostat circuitry associated with electrodes of the first chip device and configured for operating electrochemical impedance spectroscopy (EIS), and at least one second potentiostat circuitry associated with electrodes of the second chip device and configured for operating at least one of voltammetry and amperometry measurement.

In yet some further embodiments, the biosensor chip system of the present disclosure comprises a plurality of one or more first chip devices and one or more second chip devices located in a plurality of separated measurement chambers, the respective pluralities of electrodes comprise a plurality of working electrodes, reference electrode, and counter electrodes. It should be noted that the device comprises a potentiostat circuitry and a multiplexer device configured to selective transfer signals between the respective pluralities of electrode to the potentiostat circuitry.

As noted above, the biosensor chip system in accordance with the preset disclosure is particularly usable for identifying and/or quantifying and/or monitoring at least one target in a sample. In some embodiments, such target may be at least one small molecule compound. In more specific embodiments, the biosensor chip system of the present disclosure may be particularly useful for detecting small molecule compounds that may comprise at least one toxin.

In yet some further particular and non limiting embodiments, such toxin may be any toxin produced by at least one bacterial cell. In some specific embodiments, the biosensor chip system of the present disclosure may be particularly applicable for detecting, monitoring and/or quantitating at least one toxin, specifically small molecule toxin produced by cyanobacteria.

Thus, in some specific embodiments, the biosensor chip system disclosed herein is applicable for detecting, monitoring and/or quantitating at least one toxin that may be at least one cyanotoxin.

Still further, in some embodiments, the biosensor chip system disclosed herein is applicable for detecting, monitoring and/or quantitating any cyanotoxin, for example, any cyanotoxin of any group, specifically, at least one of: at least one cyclic peptide, at least one alkaloid and at least one lipopolysaccharide, or any combinations thereof.

Still further, the cyanotoxin detectable by the biosensor chip system of the present disclosure is at least one cyclic peptide. In yet some further embodiments, the cyclic peptide is at least one microcystin (MC), and at least one nodularin (NOD).

In some embodiments, the biosensor chip system of the present disclosure is applicable for detecting, monitoring and/or quantitating at least one toxin that is at least one microcystin. More specifically, in some embodiments, the microcystin is at least one of Microcystin-leucine-arginine (MC-LR), Microcystin-arginine-arginine (MC-RR), Microcystin-tyrosine-arginine (MC-YR), and Microcystin-leucine-alanine (MC-LA), and any combination, derivatives and variants thereof.

In some specific embodiments, the biosensor chip system according to the present disclosure is applicable for detecting, monitoring and/or quantitating at least one microcystin, specifically, Microcystin-LR (MC-LR), or any derivatives and variants thereof. Thus, the invention provides at least one biosensor chip system specifically applicable for detecting, monitoring and/or quantitating MC-LR.

In yet some further embodiments of the biosensor chip system of the present disclosure, at least one of: (i) the at least one working electrode of the first chip device is connected directly or indirectly to at least one antibody that specifically binds the at least one cyanotoxin; and (ii) the at least one working electrode of the second chip device is connected directly or indirectly to the at least one cyanotoxin. Thus, the disclosed system may comprise working electrode/s that ae connected to antibody that recognizes the specific cyanotoxin, and operates via EIS, or alternatively, and/or additionally, working electrodes that are bound to cyanotoxin itself, and are operated via voltammetry or amperometry, and work in some embodiments in a competitive assay as discussed herein after.

In some embodiments, the biosensor chip system of the present disclosure is applicable for any sample, specifically, any of the samples disclosed by the present disclosure. In some embodiments, the sample is an environmental sample or a biological sample.

A further aspect of the present disclosure relates to a kit comprising: In component (a), at least one biosensor chip system usable for identifying and/or quantifying and/or monitoring at least one target in a sample. The system of the disclosed kit comprises at least one of: at least one first and at least one second chip devices. Thus, in some embodiments, the kit may comprise a system comprising at least one of the first device, in yet some alternative embodiments the kit disclosed herein may comprise at least one second device. In yet some alternative embodiments, the disclosed kits may comprise at least one system comprising at least one first device and at least one second device. In more specific embodiments, the first chip device of the system of the disclosed kit comprises a first plurality of electrodes connectable to at least one electronic device. At least one of the electrodes is a working electrode. The working electrode is connected directly or indirectly to at least one target binding site and/or moiety. It should be noted that the target binding site and/or moiety specifically binds the at least one target or any component thereof. In some embodiments, the plurality of electrodes is configured for electrochemical impedance spectroscopy (EIS) analysis of said sample. Still further, the second chip device comprises: a second plurality of electrodes connectable to at least one electronic device. The at least one of the electrodes is a working electrode. More specifically, the working electrode is connected directly or indirectly to the target or any component thereof. In yet some further embodiments, the plurality of electrodes is configured for electrochemical voltammetry or amperometry analysis of the sample. Still further, in some embodiments, the kit may further comprises at least one of: (b), at least one control sample and/or control standard value; and (c), instructions for use.

In some embodiments, the at least one biosensor chip system of the disclosed kit is as defined by the present disclosure.

A further aspect of the present disclosure relates to a method for identifying and/or quantifying and/or monitoring at least one target in a sample. More specifically, the method comprising at least one of the following steps: (a) performing an electrochemical impedance spectroscopy (EIS) analysis of the sample, and/or (b), performing an electrochemical voltammetry or amperometry analysis of the sample. More specifically, the disclosed method may comprise the step of performing an electrochemical impedance spectroscopy (EIS) analysis of the sample. In some embodiments, performing such analysis comprises: in a first step (i), contacting with the sample a first plurality of electrodes comprising at least one working electrode and at least one reference electrode, or any first chip device or system comprising the same. It should be noted that the at least one working electrode is connected directly or indirectly to at least one target binding site and/or moiety. The next step (ii), involves applying voltage signal between the at least one working electrode and the at least one reference electrode, and determining electrical current between the electrodes in response to the voltage signals for a selected number of one or more signal frequencies; and (iii), determining relations between electrical current response and voltage signal for the one or more signal frequencies; and determining electrical impedance between the at least one working electrode and the at least one counter electrode. It should be noted that the impedance variation being indicative of presence and/or quantity of the at least one target in the sample.

As discussed herein, the methods of the present disclosure may comprise either as an alternative step, or as an additional step, (b), performing an electrochemical voltammetry or amperometry analysis of the sample. In some embodiments, such additional and/or alternative analysis comprising: (i), contacting with the sample a second plurality of electrodes comprising at least one working electrode and at least one reference electrode, or any second chip device or system comprising the same. In some embodiments, at least one working electrode is connected directly or indirectly to the at least one target or any component thereof. Accordingly, the sample further comprises at least one first binding molecule specific for the at least one target, and at least one second binding molecule specific for the first binding molecule. It should be noted that the second binding molecule comprises at least one labeling moiety that comprises and/or produces at least one electroactive product.

Still further, the next step (ii), applying voltage signal between the at least one working electrode and at least one reference electrode and determining electrical current through the at least one working electrode in response to varying voltage signal; and (iii) determining peak current value, the peak current value is inversely indicative of presence and/or quantity of the at least one target.

In some embodiments, where the sample is subjected to an electrochemical impedance spectroscopy (EIS) analysis. Accordingly, the method further comprising processing electrical impedance determined based on one or more voltage signal frequencies for determining charge transfer electrical resistance between the at least one working electrode and the at least one counter electrode, and determining presence of the at least one target in the sample whenever said charge transfer electrical resistance is greater than a predetermined threshold value.

Still further, determining the charge transfer electrical resistance comprises determining an electrical circuit model representing charge transfer between the electrodes, the electrical circuit may comprise capacitance model connected in parallel to inductance model and charge transfer electrical resistance model, thereby allowing to determine charge transfer electrical resistance in accordance with total impedance of the circuit.

In yet some further embodiments, where the method alternatively, or additionally, comprises subjecting wherein said sample is subjected to an electrochemical voltammetry or amperometry analysis, the method further comprises applying the peak current value determined for the sample on a predetermine standard curve for determining concentration of said at least one target in the sample.

According to such embodiments, where the second device and/or systems thereof is used by the methods of the invention, the at least one labeling moiety of the at least one second binding molecule, produces at least one electroactive product.

Still further, such labeling moiety of the second binding molecule added to the sample, comprises at least one enzyme that catalyzes the conversion of at least one substrate into at least one electroactive product.

In some embodiments of the disclosed methods, the enzyme is at least one of horseradish peroxidase (HRP), and alkaline phosphatase (ALP).

Still further, in some embodiments, the enzyme is HRP that catalyzes the oxidation of at least one substrate. More specifically, the at least one of the substrates of this enzyme is acetaminophen.

Thus, in some embodiments, the method, when using systems that comprise the second device, may further comprise the step of adding or providing the sample with an effective amount of acetaminophen.

In yet some alternative embodiments of the disclosed methods, the second binding molecule provided with the sample where systems that comprise the second device are used, the at least one labeling moiety of such at least one second binding molecule comprises at least one electroactive product. In some specific embodiments, such labeling moiety is at least one Ferrocene molecule.

Still further, in some embodiments, the at least one first binding molecule is at least one primary antibody specific for the at least one target, and the at least one second binding molecule, is at least one secondary antibody specific for the primary antibody.

In some embodiments, the methods of the present disclosure are specifically applicable for identifying and/or quantifying and/or monitoring at least one target in a sample. In some embodiments, such target may be at least one small molecule compound.

In more specific embodiments, the methods of the present disclosure may be particularly useful for detecting small molecule compounds that may comprise at least one toxin.

Thus, in some specific embodiments, the methods disclosed herein is applicable for detecting, monitoring and/or quantitating at least one toxin that may be at least one cyanotoxin.

Still further, in some embodiments, methods disclosed herein is applicable for detecting, monitoring and/or quantitating any cyanotoxin, for example, any cyanotoxin of any group, specifically, at least one of: at least one cyclic peptide, at least one alkaloid and at least one lipopolysaccharide, or any combinations thereof.

Still further, the cyanotoxin detectable by the methods of the present disclosure is at least one cyclic peptide. In yet some further embodiments, the cyclic peptide is at least one microcystin (MC), and at least one nodularin (NOD).

In some embodiments, the methods are applicable for detecting, monitoring and/or quantitating at least one toxin that is at least one microcystin. More specifically, in some embodiments, the microcystin is at least one of Microcystin-leucine-arginine (MC-LR), Microcystin-arginine-arginine (MC-RR), Microcystin-tyrosine-arginine (MC-YR), and Microcystin-leucine-alanine (MC-LA), and any combination, derivatives and variants thereof.

In some specific embodiments, the methods according to the present disclosure is applicable for detecting, monitoring and/or quantitating at least one microcystin, specifically, Microcystin-LR (MC-LR), or any derivatives and variants thereof. Thus, the invention provides methods using at least one biosensor chip system specifically applicable for detecting, monitoring and/or quantitating MC-LR.

In yet some further embodiments of the methods of the present disclosure, at least one of: (i) the at least one working electrode of the first chip device used by the methods is connected directly or indirectly to at least one antibody that specifically binds the at least one cyanotoxin; and (ii) the at least one working electrode of the second chip device used b the methods is connected directly or indirectly to the at least one cyanotoxin. Thus, the disclosed system may comprise working electrode/s that ae connected to antibody that recognizes the specific cyanotoxin, and operates via EIS, or alternatively, and/or additionally, working electrodes that are bound to cyanotoxin itself, and are operated via voltammetry or amperometry, and work in some embodiments in a competitive assay as discussed herein after.

In some embodiments, the methods of the present disclosure are applicable for any sample, specifically, any of the samples disclosed by the present disclosure. In some embodiments, the sample is an environmental sample or a biological sample.

In some embodiments, the environmental sample comprises at least one sample obtained from natural or artificial water reservoir, reclaimed water, and wastewater treatment and sewage treatment.

Still further, in some embodiments, the method of the present disclosure are performed using any of the systems defined by the present disclosure.

A further aspect of the present disclosure relates to a method of treating, preventing, ameliorating, reducing or delaying the onset of a disorder associated with exposure to at least one toxin in a subject in need thereof. In some embodiments, the method comprising: First in step (a), classifying a subject as exposed to the toxin if the presence of the at least one toxin is determined in at least one biological sample of the subject, or in at least one environmental sample associated with the subject. In some embodiments, determination of the presence of the at least one toxin in the sample is performed by at least one of: (I) performing an electrochemical impedance spectroscopy (EIS) analysis of the sample, and/or (II), performing an electrochemical voltammetry or amperometry analysis of the sample. More specifically, in some embodiments, for classifying the subjects, the disclosed method may comprise the step of (I) performing an electrochemical impedance spectroscopy (EIS) analysis of the sample. In some embodiments, performing such analysis comprises: in a first step (i), contacting with the sample a first plurality of electrodes comprising at least one working electrode and at least one reference electrode, or any first chip device or system comprising the same. It should be noted that the at least one working electrode is connected directly or indirectly to at least one target binding site and/or moiety. The next step (ii), involves applying voltage signal between the at least one working electrode and the at least one reference electrode, and determining electrical current between the electrodes in response to the voltage signals for a selected number of one or more signal frequencies; and (iii), determining relations between electrical current response and voltage signal for the one or more signal frequencies; and determining electrical impedance between the at least one working electrode and the at least one counter electrode. It should be noted that the impedance variation being indicative of presence and/or quantity of the at least one target in the sample.

As discussed herein, in some embodiments, for classifying the subjects, the disclosed method may comprise either as an alternative step, or as an additional step, (II), performing an electrochemical voltammetry or amperometry analysis of the sample. In some embodiments, such additional and/or alternative analysis comprising: (i), contacting with the sample a second plurality of electrodes comprising at least one working electrode and at least one reference electrode, or any second chip device or system comprising the same. In some embodiments, at least one working electrode is connected directly or indirectly to the at least one target or any component thereof. Accordingly, the sample further comprises at least one first binding molecule specific for the at least one target, and at least one second binding molecule specific for the first binding molecule. It should be noted that the second binding molecule comprises at least one labeling moiety that comprises and/or produces at least one electroactive product. Still further, the next step (ii), applying voltage signal between the at least one working electrode and at least one reference electrode and determining electrical current through the at least one working electrode in response to varying voltage signal; and (iii) determining peak current value, the peak current value is inversely indicative of presence and/or quantity of the at least one target.

The next step (b), of the disclosed therapeutic methods administering to a subject classified as an infected subject in step (a), a therapeutically effective amount of at least one anti-toxin agent and/or additional therapeutic agent.

In some embodiments, determination of the presence of the at least one toxin in the sample is performed by the method as defined by the invention, and specified herein above.

In some embodiments, the toxin is cyanotoxin, preferably, MC-LR. Thus, the therapeutic methods may be applicable for treating disorders associated with exposure to the MC-LR. In some embodiments, such disorders may comprise at least one of liver damage, renal failure and neoplastic disorders.

Still further aspect of the present disclosure relates to a method for identifying and/or quantifying at least one cyanotoxin in a sample, the method comprising:
contacting the sample with at least one working electrode, at least one reference electrode, and at least one counter electrode, or any biosensor chip or kit comprising said electrodes, wherein the at least one working electrode is connected directly or indirectly to at least one cyanotoxin binding site and/or moiety;
measuring electrical voltages between the at least one working electrode and said at least one reference electrode in response to electric currents of different frequencies applied between said at least one working electrode and the at least one reference electrode;
determining electrical impedances based on the measured electrical voltage and the electric currents applied at the different frequencies;
determining a charge transfer electrical resistance based on the determined impedances; and
   determining presence of the at least one cyanotoxin in the sample whenever said charge transfer electrical resistance is greater than a predetermined threshold value.

These and other aspects of the invention will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1B. mAb-EspB-B7 binds EspB with high affinity

FIG. 1A. mAb-EspB-B7 binding affinity to purified EspB was evaluated by ELISA. A 96-well plate coated with EspB was incubated with serially diluted mAb-EspB-B7. mAb-EspB-B7 binding was determined using anti-human IgG HRP-conjugated antibody. Error bars represent±SD.

FIG. 1B. SPR sensorgrams of mAb-EspB-B7 binding to an EspB-coated chip. mAb-EspB-B7 was added at various concentrations between 10 and 90 nM. Sensorgrams were fitted to the steady-state model.

FIG. 2A-2B. mAb-EspB-B7 binds to recombinant and native EspB

FIG. 2A. EPEC wild type (WT), ΔescN, ΔespB and ΔespB expressing EspB-His strains were grown under T3SS-inducing conditions for 6 hr. The bacterial pellets and supernatants were separated and analyzed using SDS-PAGE and western blotting with mAb-EspB-B7. EspB expression within the bacteria (pellet) was observed only for the ΔespB+EspB-His strain, while EspB secretion (supernatant) was observed for both WT EPEC and the complemented ΔespB+EspB-His strain.

FIG. 2B. EPEC WT, ΔescN, ΔespB and ΔespB+EspB-His bacteria were grown under T3SS-inducing conditions for 3 hr. Thereafter, 1×10⁷ bacteria were incubated with mAb-EspB-B7, washed, and stained with Alexa Fluor 488 goat anti-human IgG antibody. Flow cytometry analysis was performed on a Gallios instrument (Beckman coulter).

FIG. 3A-3C. mAb-EspB-B7 binding to EspB under various conditions mAb-EspB-B7 binding to EspB was evaluated by ELISA.

FIG. 3A. shows evaluation of the binding in different media.

FIG. 3B. shows evaluation of the binding under various pH conditions.

FIG. 3C. shows evaluation of the binding at different NaCl concentrations. Error bars represent±SD.

FIG. 4. mAb-EspB-B7 is thermally stable

The melting temperatures (Tₘ) of mAb-EspB-B7 alone or in combination with recombinant EspB were determined by nano Differential Scanning Fluorimetry (nanoDSF), Prometheus NT.48, NanoTemper.

FIG. 5. mAb-EspB-B7 does not interfere with the EspB-EspD interaction

Supernatants of EPEC ΔespD expressing EspD-³⁵His were purified using Ni-NTA beads. EPEC ΔespD strain without the pEspD-³⁵His expression vector, was used as a negative control. Samples of supernatants (S) and elution (E) fractions were loaded on SDS-PAGE and analyzed by western blotting with mouse anti-His and anti-EspB antibodies (to avoid detection of the human EspB antibody). Analysis of the supernatants confirmed EspB and EspD secretion into the extracellular medium. The co-elution of EspB with EspD-³⁵His was not affected by the absence or presence (100 nM and 200 nM) of mAb-EspB-B7. Low EspB non-specific binding to the Ni-NTA beads was detected (in the absence of EspD-³⁵His).

FIG. 6A-6D. mAb-EspB-B7 epitope mapping

FIG. 6A. An EspB pepstar peptide array of 78 cyclic peptides (15-residue long peptides with an 11-residue overlap) was examined for mAb-EspB-B7 binding. Image analysis was carried out with Genepix Pro 6.0 analysis software (Molecular Devices) to detect antibody binding; fluorescence signals were normalized showing their relative intensities. The putative binding site of mAb-EspB-B7 along the EspB protein is marked in light gray. Arrows indicate the signals obtained from peptides #49 and #50, which displayed the highest signal intensities. The EspB amino acid sequence in the figure is denoted by SEQ ID NO. 40.

FIG. 6B. shows mAb-EspB-B7 binding to EspB following pre-incubation with peptide #49 and peptide #49 scrambled.

FIG. 6C. shows mAb-EspB-B7 binding to EspB following pre-incubation with peptide #50 and peptide #50 scrambled

FIG. 6D. shows mAb-EspB-B7 binding to EspB following pre-incubation with peptide #49+50. The binding was evaluated by competitive ELISA and detected using anti-human IgG HRP-conjugated antibody. Peptide #78 was used as a negative control. Error bars represent±SD.

FIG. 7A-7D. mAb-EspB-B7 binding to EspB peptides

FIGS. 7A. shows mAb-EspB-B7 binding to peptide #49 and peptide #49 scrambled (SEQ ID NO. 33, and 34, respectively).

FIG. 7B. shows mAb-EspB-B7 binding to peptide #50 and peptide #50 scrambled (SEQ ID NO. 35, 36, respectively).

FIG. 7C. shows mAb-EspB-B7 binding to peptide #49+50 (SEQ ID NO. 37). mAb-EspB-B7 binding to the various peptides was evaluated by ELISA. A 96 well plate was coated with the peptides before being incubated with serially diluted mAb-B7 and detected using anti-human IgG HRP-conjugated antibody. Peptide #78 (SEQ ID NO. 38), that was used as a negative control. Error bars represent+/−SD.

FIG. 7D. Sequences of peptides #49, #49 scrambled (SEQ ID NO. 33, 34, respectively), #50, #50 scrambled (SEQ ID NO. 35, 36, respectively), #49+50 and #78 (SEQ ID NO. 37, 38, respectively). Each peptide was synthesized with the addition of cysteine residues at the C and N-termini, to enable peptide cyclization.

FIG. 8A-8B. mAb-EspB-B7 binds EspB homologs in other T3SS-expressing bacteria FIG. 8A. Wild type and mutant EPEC, EHEC, C. rodentium and Salmonella were grown under T3SS-inducing conditions. EPEC, EHEC and C. rodentium mutant strains contain a deletion in the escN gene, while Salmonella contains a deletion in the invA gene, which results in non-functional T3SSs in these mutants. The bacterial cultures were centrifuged, and the supernatants were collected, normalized, and analyzed by SDS-PAGE and western blotting using mAb-EspB-B7.

FIG. 8B (B-1 to B-3). Amino acid sequence alignment of EspB from EPEC (SEQ ID NO. 40) with C. rodentium (SEQ ID NO. 46), EHEC (SEQ ID NO. 45), or Salmonella (SEQ ID NO. 47) EspB homologs. The dark bars and/or dots represent identical, amino acids in each corresponding sequence, the different residues are indicated. The mAb-EspB-B7 epitope is annotated above the amino acids that are part of the epitope.

FIG. 9A-9B. mAb-EspB-B7 does not inhibit EPEC translocation activity into HeLa cells.

FIG. 9A. Scheme of the effector translocation assay. Infection of HeLa cells with EPEC was monitored by detecting the degradation profile of JNK, a human kinase that is subjected to cleavage by the EPEC effector, NleD.

FIG. 9B. HeLa cells were infected with wild-type (WT) EPEC in the presence or absence of 400 nM mAb-EspB-B7. After 3 hr, cells were washed, and host cell proteins were extracted and subjected to western blot analysis using anti-JNK and anti-actin (loading control) antibodies. JNK and its degradation fragments are indicated at the right of the gel. Degradation of JNK was evident in the WT EPEC, sample but not in the uninfected sample or in the samples infected with EPEC ΔescN. HeLa cells infected with WT EPEC in the presence of 400 nM mAb-EspB-B7 showed a JNK degradation profile similar to that of WT EPEC in the absence of mAb-EspB-B7.

FIG. 10A-10C. Electrochemical chip device

The figure schematically illustrates an electrochemical chip device configuration for detection of cell (e.g., EPEC) suspension based on EIS techniques according to some possible embodiments.

FIG. 10A. shows the electrochemical chip device and a sample collector.

FIG. 10B shows an exploded view of the chip device.

FIG. 10c shows a sectional view of the Chip device.

FIG. 11. The device Scheme

The figure shows a possible embodiments of an electrochemical chip device packaged in a chamber along with an inlet “rough” filter (2 μm) and an outlet fine filter (500 nm). Once a sample collector perforates the seal an integrated syringe plunger is operated, extracting bacteria cells from the sampler towards the measurement chamber. The microelectrode array is connected through pads that are perpendicular to the package and are inserted into a ‘dongle-like’ potentiostat device. The measurement is handled by e.g., a smartphone application displaying electrochemical impedance spectroscopy (EIS) readouts, which is also responsible for data acquisition and storage, and is potentially capable of uploading the results to a designated cloud (not shown).

FIG. 12A-12E. mAb-EspB-B7-based impedimetric biosensor

Figure shows schematically illustrates a biosensor (e.g., mAb-EspB-B7-based impedimetric biosensor), and cell suspension measurement conducted therewith, according to some possible embodiments;

FIG. 12A. demonstrates EIS-based detection of whole bacterial EPEC cells. In this non-limiting example electrochemical chips (with a working electrode e, radius of about 0.3 mm, counter electrode e_{c} having radius of about 0.6 mm, and a square reference electrode eᵣ having surface area of about 0.25 mm², and respective contact pads 13w,13c,13r electrically connecting thereto) fabricated in/on a substrate (13) using microelectronic fabrication technologies and are subsequently modified with a thiolated mAb-EspB-B7 using thiol-gold chemistry. The electrodes e_{w}, eᵣ, e_{c} are sealably enclosed inside an electrochemical cell structure, configured to receive a sample. The immobilization of mAb-EspB-B7 and capture of antigen affect the impedance measured between the underlying electrodes. An EIS measurement thus allow for the interrogation of the electrochemical system and separation of the individual components that affect the electrochemical cell circuit established by introducing the sample into the electrochemical cell (ci). The generated Nyquist plot is fitted to an equivalent circuit from which the different resistance values are extracted (inset).

FIG. 12B. Shows the Nyquist plots obtained from measurements of a bare gold working electrode (bare GE), from the working electrode after the immobilization of mAb-EspB-B7 (GE+mAb) thereon, and the mAb-EspB-B7-coated working electrode after incubation with 250 μg/mL purified EspB protein (GE+mAb+EspB).

FIG. 12C. Shows relative R_{ct} (charge transfer resistance) values of purified EspB protein (1, 4, 10 and 250 μg/ml) demonstrating a dose-dependent increase in the detected R_{ct} values. Relative R_{ct} values are the means of the R_{ct} ratios (before and after antigen capture) calculated from 3-6 measurements. Error bars represent the ±SD.

FIG. 12D. Shows that the change in the detected R_{ct} values is exponentially dependent on EspB concentration.

In FIG. 12E. specific binding of WT EPEC cells is indicated, resulting in a larger contribution to R_{ct} compared with the ΔespB null strain. The percent change in R_{ct} ratios measured for EPEC WT and ΔespB was calculated and averaged from 20 repeats (five measurements each containing four samples) for each strain. The mean of the averaged ratios and the standard error of the mean were calculated.

FIG. 13. Electrochemical cell device

The figure shows schematically illustrates an electrochemical cell device (ci) with a potentiostat (PS) and connection thereof to a computer device (e.g., smartphone), demonstrating how the binding of the EPEC cells to the mAb-EspB-B7 coated working electrode affects the EIS measurements.

FIG. 14. Electrochemical cell device

The figure shows modification of a gold electrode (or any other suitable electrically conducting metal or carbon, or other conductive polymeric material that can be used as a working electrode in an electrochemical setup) with anti-pathogenic E. coli monoclonal antibodies such as: anti-EspB or others specific mAb's, and the impedance response measured over a predefined frequency range, according to possible embodiments. The impedance spectra is fitted to an electric circuit (right). Specific binding of antigens affects certain circuit parameters and enable detection and quantification of the bound antigen.

FIG. 15. The determination process

The figure illustrates cell suspension determination process according to is the indicated flowchart.

FIG. 16A-16D. electrochemical cells chip device

These figures schematically exemplify selected configuration of electrochemical cells chip device configured with plurality of electrode arrangement according to some possible embodiments.

FIG. 16A. shows a chip configuration (60) comprising a plurality of electrochemical cells (C₁, C₂, . . . Cₙ) and respective plurality of electronic circuitries (65) electrically connected thereto.

FIG. 16B shows a chip configuration (69) comprising a plurality of working electrodes (e₁, e₂, . . . eₙ) enclosed inside a single electrochemical cells (cᵢ) operated using a single electronic circuitry (65).

FIGS. 16C and 16D. show the exploded and assembled chip configuration configured with a plurality of working and reference electrode and a common counter electrode.

FIG. 17A-17D. EIS-based biosensor for MC-LR detection, and fabrication thereof

FIG. 17A. Schematic illustration of the developed biochip. Multiple electrochemical cells are fabricated by microelectronic manufacturing techniques. Anti-MC-LR monoclonal antibodies are chemically modified and covalently immobilized to an activated gold working electrode surface. The biochip is interfaced with a portable potentiostat device (a generalized circuit diagram is shown on the left). Exposure to a water sample contaminated with MC-LR-secreting cyanobacteria results in specific binding of the toxins to the electrode-bound antibodies, affecting the electrode's impedance. This change can be measured and analyzed in real-time, allowing the quantification of toxins in the sample.

FIG. 17B. An EIS measurement is used to interrogate the electrochemical system and separate the individual components that affect the circuit. A Nyquist plot depicts the change in the “real” component of the impedance (Z′ or Zᵣₑₐₗ) versus the “imaginary” component (Z″ or −Z_{imag}, which results from capacitance) over a wide range of frequencies. The generated Nyquist plot is fitted to an equivalent circuit from which the different resistance values are extracted (inset). Solution resistance, Rₛ, charge transfer resistance, R_{ct}, Warburg resistance, Z_{w}, and double layer capacitance, C_{dl}, can all be modeled and calculated.

FIG. 17C. process flow of chip fabrication by photolithography and sputtering: (a) The wafer is cleaned with acetone, isopropanol, and distilled water; (b) Photoresist (PR) coat is spun onto the wafer and soft baked. (c) Patterns are projected onto the wafer (photolithography); (d) The substrate is developed and unexposed PR is removed. (e) Titanium and gold are sputtered onto the substrate (f) The PR and gold are removed by a lift-off process. Following this, the wafer is rinsed with ACT, IPA, and DI, and (g) The wafer is ready for electroplating.

FIG. 17D. Following fabrication (and surface characterization of the deposited electrodes), the reference electrodes are electroplated. Briefly, the formation of a reference electrode is carried out by electroplating silver (from a silver plating bath) followed by anodic generation of a silver chloride layer to obtain a silver/silver chloride layer (Ag/AgCl). The electroplating of silver yields a typical white luster deposit that appears, in a SEM analysis, as a homogenous crystalline deposit with denseAg nuclei of ˜1 μm (Bar: 5 μm).

FIG. 18A-18B. The biosensing platform

FIG. 18A. Silicon-based electrochemical chips are microfabricated using photolithography and metal deposition.

FIG. 18B. Custom manufactured apparatus. Image of a machined PTFE apparatus providing electrical contacts to electrochemical chips and chambers for interrogating multiple samples.

FIG. 19. Characterization of the EC cell

Verification of a newly formed Ag/AgCl reference electrode is carried out by measuring its potential versus a commercial reference electrode in varying electrolyte (NaCl) concentrations. The response of the electrode is plotted against the log[NaCl] such that any log change in Cl concentration is expected to yield a 59 mV potential difference, according to the Nernst equation. In practice, deviations from this value are expected to evolve from the nature of the measured electrode (an open reference electrode), the quality differences, and experimental conditions (mainly varying distances between the measuring electrodes that affect solution resistance). The disclosed reference electrodes demonstrate a ‘Nernstian behavior’, close to the theoretical value.

FIG. 20A-20C. Characterization of the EC cell

Verification of the whole cell is obtained by i-E curve (cyclic voltammogram) with the redox couple ferricyanide.

FIG. 20A. CV at different scan rates with a solution of 20 mM ferricyanide/ferrocyanide. 4 different scan rates were used, consecutively.

FIG. 20B. Corresponding analysis obtained from the biochip. The peak height increased as the scan rate increased and was linearly proportional to the square root of the scan rate, showing the anodic peaks (top) and cathodic (bottom).

FIG. 20C. peak separation is relatively independent of scan rate.

FIG. 21A-21B. Biofunctionalization of EC chips

FIG. 21A. Immobilization of antibodies is based on covalent attachment using well-established gold-thiol chemistry. Antibodies were thiolated by using the thiolating reagent 2-imminothiolane hydrochloride (Traut's reagent), which reacts with primary amines (—NH₂) to introduce sulfhydryl (—SH) groups while maintaining charge properties similar to the original amino group. The reaction was optimized to obtain an average of ˜6 —SH group per antibody. FIG. 21B. Ellman assay using DTNB (left) was used to assess the thiolation efficiency. The reaction is monitored by a spectrophotometer.

FIG. 22A-22F. Surface characterization of functionalized electrodes

FIG. 22A-22D. Assessment of thiolated antibodies immobilization to the gold working electrode is carried out by fluorescence microscopy analysis. Thiolated Cy3-labeled antibody is incubated on the gold WE. As a control, a non-thiolated Cy3 antibody was used. Incubation is followed by rigorous rinsing of the electrodes.

FIG. 22E-22F. AFM image of gold working electrode surface before and after the covalent immobilization of thiol-modified antibodies.

FIG. 23. EIS response of the developed immunosensor

The impedance spectra of a bare electrode (‘bare GE’) are characterized by low charge transfer resistance (R_{ct}) and high Warburg (Z_{w}) impedance. After antibody immobilization (‘GE+mAb’), the R_{ct} increases, and the Z, is no longer dominant. Following the binding of the toxins (‘3 μg/L’), the R_{ct} increases dramatically. This increase is proportional to the concentration of the bound toxin and allows its quantification in the sample.

FIG. 24. Nyquist plots obtained from antibody-functionalized electrodes following incubation with MC-LR

Change in R_{ct} signal following MC-LR binding to MC10E7/GE at different incubations times was evaluated. Measurements conducted in PBS pH 7.4 containing 10 mM Fe(CN)₆^{4-/3-} and 0.1 M KCl show the dependence of impedimetric response (R_{ct}) on immunoreaction time. Bar plots (change in R_{ct} response) were calculated from the ratio of MC-LR/MC10E7/GE and MC10E7/GE normalized to 1 (error bars: SEM, n=3).

FIG. 25A-25D. MC-LR detection with an impedimetric immunosensor

FIG. 25A. The obtained Nyquist plots from measurements of a bare gold electrode (‘bare GE’), electrode after the immobilization of anti-MC-LR mAb (‘GE+mAb’), and after incubating with six different concentrations of purified MC-LR toxin: 0.0003, 0.003, 0.03, 0.3, 3, and 30 μg/L. (The lowest concentration yielded a similar impedimetric signal as the background).

FIG. 25B. Relative R_{ct} values of purified MC-LR toxin protein demonstrating a dose-dependent increase in R_{ct}.

FIG. 25C. An exponential increase in R_{ct} is observed. Inset shows a linear dependence at lower concentrations, yielding a calibration curve for target MC-LR.

FIG. 25D. Detection of MC-LR from cyanobacterial suspensions is feasible with the developed biosensor. Specific binding of MC-LR, contributing to an increase in R_{ct} is indicated with Microcystis suspensions, whereas no response was observed with Spirulina suspensions. Higher signals were obtained from filtered Microcystis suspension, as expected. Incubation of MC-LR on an electrode functionalized with an unrelated antibody (mAb-EspB-B7), showed no MC-LR binding, further supporting the specificity of the biosensor. The changes in R_{ct} values (% ΔR_{ct}) are the means of the R_{ct} ratios (before and after antigen-capture), calculated from triplicates. The error bars represent±SD.

FIG. 26A-26B. The ic-ELISA for Microcystin-LR detection

FIG. 26A. Different concentrations of MC-LR were detected by ic-ELISA ranging from 0.03 μg/L to 30 μg/L (error bars: SD, n=3).

FIG. 26B. The standard curve obtained from ic-ELISA measured in 8 repeats of ELISA plate wells that were coated with 3 μg/mL MC-LR toxin. The antibody MC10E7 dilution was 1:3,000; enzyme Immunoconjugate dilution was 1:4,000. The experimental data are shown as a discrete plot with error bars in black. The solid black curve is a fit of the Hill equation to the experimental data using OriginLab. The inset image shows the range of quantitative detection with good linearity.

FIG. 27A-27B. Raw cyanobacterial cultures used as a model for contaminated water

FIG. 27A. figure shows whole bacterial cell suspensions of Microcystis aeruginosa PPC 7806.

FIG. 27B. figure shows whole bacterial cell suspensions of Spirulina sp. Both samples were cultured, grown, and maintained in BG-11 at a temperature of 24-26° C. and light intensity of 6 μmol photons m⁻²s⁻¹.

FIG. 28A-28B. Assessment of the specificity of the impedimetric immunosensor

FIG. 28A. The obtained Nyquist plots from measurements of a bare gold electrode (‘bare GE’), electrode after the immobilization of mAb-EspB-B7 (‘GE+mAb-EspB-B7’), and after incubation with 2 μg/mL purified MC-LR toxin. Incubation of MC-LR on an electrode functionalized with a nonspecific antibody (mAb-EspB-B7), did not affect the impedimetric signal, indicating no MC-LR binding.

FIG. 28B. Nyquist plots from measurements of a bare gold electrode, electrode after the immobilization of anti-MC-LR mAb (GE+mAb), and after incubation with Spirulina suspensions. No response was observed with Spirulina suspensions. These two measurements provide further support for the specificity of the biosensor.

FIG. 29. Schematic illustration of the indirect competitive ELISA

Antibodies (mAbs) specific to MCs are incubated with the antigen to be measured in the raw sample. The formed antigen-antibody (Ag-Ab) complexes with free and unbound mAbs are added to a well plate-coated MC-LR toxin, and the free mAbs bind to the adsorbed MC-LR on the plate well. Following incubation and washing, HRP-conjugated secondary antibody is added followed by a substrate allowing the enzymatic electro-active product to produce a color that can be measured using an ELISA plate reader.

FIG. 30A-30E. Schematic description of the steps involved in the development of the amperometric biosensor for the ECI assay

FIG. 30A. Mercaptoundecanoic acid (MUA) modified gold electrode surface.

FIG. 30B. EDC/NHS activated MUA gold electrode surface.

FIG. 30C. MC-LR immobilized on an activated gold sensor surface followed by BSA blockage

FIG. 30D. Following a washing step with PBS-T, 1°-HRP-Ab conjugate complexes are added to the MC-LR-coated WE. Unbound antibodies are removed by washing. Different concentrations of MCs standard solution are incubated to the 1° Ab-HRP-Ab/BSA/MC-LR/EDC-NHS/MUA/GE modified electrode. MCs bound to free antibodies from the 1° Ab-HRP-Ab mixture bound to the adsorbed MC-LR.

FIG. 30E. Following a washing step, the HRP substrate (acetaminophen) is added. Then a potential of −100 mV is applied to allow the enzymatic electro-active product to be reduced on the WE generating measurable current.

FIG. 31A-31C. The ic-ELISA for MC-LR detection

FIG. 31A. Different concentrations of MC-LR were detected by ic-ELISA ranging from 0.03 μg/L to 30 μg/L (error bars: SD, n=3). The standard curve of ic-ELISA was measured in 8 repeats of ELISA plate wells that were coated with 3 μg/mL MC-LR toxin. The antibody MC10E7 dilution was 1:3 000; enzyme Immunoconjugate dilution was 1:4 000.

FIG. 31B. Standard curve obtained from the ic-ELISA. The experimental data are shown as a discrete plot with error bars in black. The solid black curve is a fit of the Hill equation to the experimental data using OriginLab.

FIG. 31C. Range of quantitative detection with good linearity.

FIG. 32. EC Characterization of MC-LR

Cyclic voltammograms of 8 mM Fe(CN)₆^{4-/3-} and four different concentrations of MC-LR solutions (20 and 30 μg/L) in PBS (pH 7.4) at a scan rate of 100 mV/sec.

FIG. 33A-33B. Cyclic voltammetry at a scan rate of 50 mV/sec of PBS, pH 7.4, substrate (a mixture of 0.3 mM H₂O₂, and 0.45 mM APAP), and the reaction of HRP with the substrate

FIG. 33A. Scan initiated following 1 min incubation of solution reactants. CVs were performed separately.

FIG. 33B. repeated CV cycles at a scan rate of 50 mV/sec of a solution containing 0.3 mM APAP and H₂O₂ and 0.5 μg/ml HRP. Scan initiated following 1 min incubation of solution reactants. Cycles 4-6 were initiated after a 2 minutes pause, where no potential was applied.

FIG. 34A-34B. Characterization of the electrode

Nyquist plot (FIG. 34A), and analysis using an equivalent Randles circuit (FIG. 34B) of the bare gold electrode (ge) (black square), the EDC-NHS/MUA/ge functionalized electrode (black circle) and the immobilized MC-LR toxin (black triangle) in the presence of 10 mM Fe(CN)₆^{3-/4-} in 1×PBS (pH 7.4). Impedance spectra were acquired at the formal potential of 10 mV in the 10 kHz to 0.1 Hz frequency range. The symbols represent the experimental data. The Change in R_{ct} signal following the biofunctionalization of the electrode bare gold working electrode (ge) with 11-mercaptoundecanoic acid (MUA) and EDC-NHS chemistry to activate —COOH (EDC-NHS/MUA/ge) for MC-LR binding (MC-LR/EDC-NHS/MUA/ge). Bar plots were the ratio of MC-LR/EDC-NHS/MUA/ge and EDC-NHS/MUA/ge normalized to 1 (error bars: SEM, n=5).

### DETAILED DESCRIPTION OF THE INVENTION

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

In recent years, advances in mAb discovery and production have ushered in the development of pathogen-specific mAb's to be used either per se as antibacterial drugs or to be integrated into various diagnostic platforms for the detection of specific pathogens. In the latter regard, the high affinity and specificity of mAbs are characteristics that can be exploited in diagnostic tools giving reduced false positive/negative results. Such tools could provide rapid and accurate identification of bacterial agents at POC, thus supporting better clinical management of patients and preventing the transmission of infectious diseases in the community.

The present disclosure describes a mAb raised against EspB, an essential component within the T3SS that is crucial for the infectivity of numerous Gram-negative bacteria, including EPEC. The results disclosed herein demonstrate that mAb-EspB-B7 binds EspB with nM affinity and high specificity. As commercial monoclonal antibodies against bacterial species, targeted mostly against common bacterial antigen such as the flagella or the bacterial Lipopolysaccharides (LPS), have been reported to have micromolar affinities [18], the mAb-EspB-B7 holds greater potential to allow efficient detection of bacterial pathogens due to its nM affinity.

The antibody binding to its EspB target was stable over a wide range of pH values, excluding acidic pH values, and across various salt concentrations. A reduced binding capacity was detected only under high salt concentrations (>250 mM), suggesting that the antibody-antigen binding interface is governed by electrostatic interactions. This idea is supported by the observation that the identified EspB epitope contains nearly 50% of charged amino acids, which might be involved in the antibody-antigen binding. mAb-EspB-B7 demonstrated a relatively high melting temperature, which was moderately elevated when the antibody was complexed with its antigen. This result suggests that EspB binding has a stabilizing effect on the antibody, as was previously reported for anti-ricin neutralizing antibody. Furthermore, the melting temperature profile of mAb-EspB-B7 showed three distinct events that probably correspond to the melting order of the CH2 region, followed by the Fab and CH3, as reported previously. This melting profile indicates that the mAb-EspB-B7 would be suitable for applications that require relatively high thermal stability. The rational for pinpointing EspB derived from the fact that EspB is getting exposed to the extracellular environment following EPEC entrance to the digestive system and in response to thermal and chemical signals [6]. Based on the number of T3SS complexes expressed on each bacteria and the predicted number of EspB subunits found in each T3SS complex, the inventors estimate that there are approximately 100 EspB molecules per each bacterial cell [7]. The present disclosure reports the development and characterization of mAb-EspB-B7 and further demonstrate its potential as a bio-recognition element in a reliable and easy to use electrochemical biosensor. The mAb-EspB-B7 demonstrated high specificity and affinity towards EspB, binding capacity to soluble EspB and in the context of whole bacteria, and high stability under a variety of conditions. These characteristics make mAb-EspB-B7 an excellent candidate to serve as an integral component of a mAb-based biosensor. Indeed, a biosensor based on mAb-EspB-B7 demonstrated excellent performance in recognizing both soluble EspB and in the context of the whole bacteria. Such a biosensor can be used as a powerful tool for more rapid, cost-effective, and sensitive assays that can identify infective agents at the point of care (POC).

Epitope mapping using the specially designed cyclic-peptide array of the present disclosure revealed that mAb-EspB-B7 binds mostly to a specific amino acid sequence located at positions 193-210 along the EspB sequence (SEQ ID NO. 39). In a previous study, it was shown that this region was not important for EspB-EspD interactions, a fact that was further corroborated by our observation that mAb-EspB-B7 does not disrupt the interaction between the two proteins. Moreover, the observation that mAb-EspB-B7 binds EspB as a component of the fully assembled T3SS complex supports the notion that the epitope of EspB is exposed and not buried within the EspB-EspD interface. It is noteworthy that the peptide array results also identified an additional region, corresponding to peptides #9-12 (SEQ ID NO. 48), that demonstrated mAb-EspB-B7 binding. This finding could perhaps suggest that the epitope recognized by mAb-EspB-B7 is conformational rather than linear. As the main epitope sequence (positions 193-210) is fully conserved in EPEC and C. rodentium, the lower similarity along this second region might provide an explanation for the reduced western blot signal that was observed for C. rodentium EspB (FIG. 8A). In addition, while mAb-EspB-B7 binding to a protein was observed in the supernatants of WT EHEC and C. rodentium, no binding was detected in the Salmonella supernatant. This result is in agreement with the presence of the epitope in EHEC and C. rodentium but not in Salmonella (FIG. 8B).

The ability of mAb-EspB-B7 to recognize and bind C. rodentium EspB is highly important, as it provides the scientific grounds for the use of the mAb-EspB-B7 antibody as diagnosis tool of mice infection model. In addition, while mAb-EspB-B7 did not demonstrate a reduction of bacterial infectivity in the ex vivo system, the inventors posit that examining it in a mouse model will provide a more comprehensive picture that will include the effect of the antibody in promoting certain activities of the immune system against bacteria, such as opsonization and phagocytic clearance. These activities may prevent the spread of the bacterial infection within the host body and induce a humoral response with serological memory that will shorten the infection duration, promote recovery and provide cellular and serological memory.

Another key aspect of mAb-EspB-B7 is its ability to bind both the secreted form of EspB and EspB as a component of the assembled T3SS complex within the bacterial cell. This finding provides further support for its potential as a diagnostic agent capable of detecting bacterial infections directly in clinical samples in a short time with high accuracy, as previously reported [19, 20].

Demonstrating the diagnostic potential of mAb-EspB-B7 in electrochemical biosensing is particularly interesting. Electrochemical biosensors are perfectly suited for POC diagnosis due to their inherently high sensitivity and direct electronic transduction. Direct electronic detection avoids the use of optics and light sources and allows for small form-factor devices. Moreover, bioelectrochemical sensing is indifferent to sample turbidity thus obviating the need for extensive sample purification steps. Finally, these devices are attractive since they are amenable for miniaturization and can be manufactured using conventional microelectronic fabrication techniques. The inventors developed a biochip, functionalized it with the specific mAb-EspB-B7, and applied a label-free, EIS-based detection of EspB or alternatively, EspB-presenting bacteria by simply incubating the sample for several minutes. This direct approach to electrode functionalization is advantageous compared to well-established self-assembled monolayer (SAM) generation methods since it involves a straightforward preparation and avoids a complete electrode passivation often achieved with SAM. As shown in the present disclosure, despite obvious limitations related to nonspecific adsorption and sample inhomogeneity, the biosensor provides a concentration-dependent signal that can be fit to an exponential function yielding a calibration curve. Nonlinear calibration curves have been previously reported in impedimetric biosensors [21, 22]. In addition, the inventors observed that the biosensor differentiates between T3SS-containing- and lacking-bacteria, thus providing a simple tool to detect pathogenic bacteria.

In the present disclosure, the mAb-EspB-B7 that binds with high affinity and selectivity to a T3SS-exposed protein, has been characterized and provided clear indication for using this antibody integrated into a miniaturized electrochemical biosensor to identify T3SS-containing bacteria. The mAb-EspB-B7 antibody may also be used in development of anti-bacterial drug. The present disclosure provides the use of this antibody as a part of high throughput diagnostic device, such as a portable standalone antibody-based biosensor described herein.

Therefore, in a first aspect, the present disclosure provides a biosensor system usable for identifying and/or quantifying a target in a sample. The biosensor system includes at least one of first and second chip devices, each utilizing an electrode arrangement and configured for electrochemically identifying and/or quantifying a target in a sample.

The first chip device utilizes electrochemical impedance spectroscopy (EIS) analysis of the sample. Accordingly, the first chip device includes an electrode arrangement including at least one working electrode. The working electrode is connected directly or indirectly to at least one target binding site and/or moiety, wherein said target binding site and/or moiety specifically targets said at least one target or any component thereof. The plurality of electrodes is configured for electrochemical impedance spectroscopy (EIS) analysis of said sample.

The second chip device utilizes voltammetry or amperometry analysis of the sample. The second chip device includes an electrode arrangement including at least one working electrode. The working electrode is connected directly or indirectly to at least one antibody binding site and/or moiety, wherein said antibody binding site and/or moiety comprises at least a component said target and specifically targets corresponding antibodies in the sample or any component thereof. The electrode arrangement of the second chip device is configured for voltammetry or amperometry analysis of the sample.

Generally, the biosensor chip system is formed with an enclosure defining respective measurement chambers for the first and/or second chip devices. Measurement chambers of the first chip device are generally separated from measurement chamber of the second chip device, to prevent interactions between materials used by the different chip devices.

Further, the electrode arrangements of the first and/or second chip devices are connectable to one or more electronic device enabling electrochemical analysis of the respective sample. The first chip device is configured for electrochemical impedance spectroscopy, indicating attachment of the target to the binding site on the working electrode. The second chip device is configured for voltammetry or amperometry, determining data on concentration of the target in the sample using competitive ELISA measurement techniques.

The present disclosure provides a biosensor chip device.

More specifically, a biosensor chip device usable for identifying and/or quantifying a target in a sample by electrochemical impedance spectroscopy (ETS) analysis. The chip device includes an arrangement of two or more electrodes configured to be in contact with a sample, typically within a measurement chamber. One of the two or more electrodes carries one or more binding sites, e.g., carrying antibodies such as the above described mAb-EspB-B7, or any detecting molecules applicable for the disclosed small molecules, specifically, toxins as discussed herein. Therefore, in a first aspect, the present disclosure relates to a biosensor chip system usable for identifying and/or quantifying and/or monitoring at least one target in a sample. More specifically, the system comprises at least one of: at least one first and at least one second chip devices. It should be noted that the first chip device comprises a first plurality of electrodes connectable to at least one electronic device. The at least one of the electrodes is a working electrode. More specifically, in some embodiments, the working electrode is connected directly or indirectly to at least one target binding site and/or moiety. Still further, in some embodiments, the target binding site and/or moiety specifically binds the at least one target or any component thereof. It should be further noted that the first plurality of electrodes is configured for electrochemical impedance spectroscopy (EIS) analysis of the sample. In some alternative and/or additional embodiments the system of the present disclosure may comprise in addition to the first device, or as an alternative to the first device, at least one second chip device that comprises: a second plurality of electrodes connectable to at least one electronic device. It should be noted that the at least one of the electrodes is a working electrode. The at least working electrode of the second device is connected directly or indirectly to the target or any component thereof. It should be noted that this second plurality of electrodes is configured for electrochemical voltammetry or amperometry analysis of the sample.

In some embodiments, the biosensor chip system of the present disclosure further comprises a packaging assembly configured to sealably enclose the electrodes portion of the substrate and define at least one measurement chamber encompassing the electrodes.

In some embodiments, the system disclosed herein comprises at least one of the first device. In yet some alternative embodiments, the system of the present disclosure may comprise at least one second device. In yet some alternative embodiments, the disclosed systems may comprise at least one first device and at least one second device.

Thus, in yet some further embodiments, the biosensor chip system of the present disclosure, comprises the at least one first and the at least one second chip devices. According to such embodiments, the respective pluralities of electrodes of the first and second chip devices are positioned in respective first and second separated measurement chambers.

The electrode arrangement is connectable to an electronic device for providing selected voltage variations between the two or more electrodes, enabling EIS analysis of material in the sample.

In some examples, the EIS analysis enables to determine data on one or more bacteria cells in accordance with binding of the bacteria cells to respective binding sites on the electrodes.

For example, in some embodiments the chip device comprises:
a substrate portion having a plurality of electrodes formed in an electrodes portion thereof, and
at least one electronic circuitry (e.g., potentiostat circuitry) electrically connected to said electrodes. In some embodiments, at least one of the electrodes is connected directly or indirectly to at least one target binding site and/or moiety; and
a packaging assembly configured to sealably enclose the electrodes portion of the substrate and define a measurement chamber encompassing the electrodes.

FIGS. 10A to 10C schematically illustrate an electrochemical chip device (10) configuration for sample analysis according to some embodiments of the present disclosure. The biochip (10) contains an electrochemical cell (cᵢ) configured for holding an arrangement of a micro-working electrode array in communication of sample to be inspected. the electrode array includes a plurality of two or more electrodes, typically including at least one working electrode (e_{w}), at least one reference electrode (eᵣ) and at least one counter electrode (e_{c}). The electrode array may be formed on a substrate (13) to simplify alignment and electrical connections. In some embodiments, the electrode array may be made of-Polytetrafluoroethylene (Teflon) or Acetal homopolymer (Delrin) or polypropylene, or polymethyl methacrylate or polyimide or polyvinylidene fluoride or polystyrene or other thermoplastics or heat-resistant plastic materials. The biochip (10) may generally be a part of a biosensor chip system, acting as a first chip device or a second chip device.

More specifically, FIG. 10A shows the electrochemical chip device (10) and, a sample collector (12) usable for introducing a sample into the chip device (10). The portion of the substrate (13) carrying active end of the electrodes (e_{w}, eᵣ, e_{c}) of the chip device (10) is packaged in a chamber (cᵢ) and electrical contacts of the electrodes are shown (13w,13r,13e). Generally, when acting as first chip device, the working electrode is connected to, or carrying, one or more binding sites/moieties selected to interact with one or more bacteria cells as described hereinabove. When acting as second chip device, the working electrode is connected to, or carrying, the target (e.g., molecules of the target) or any component thereof. At least the working electrode (e_{w}) may preferably be formed and/or coated by a layer of gold, to enable biofunctionalization thereof.

Electrical operation of the electrode arrangement may vary in accordance with operation as first chip device or second chip device. Generally, the first chip device operates for electrochemical impedance spectroscopy, i.e., determining impedance between electrodes in different signal frequencies. The second chip device is operating for voltammetry or amperometry measurements. These measurements use voltage (or current) variation in a (generally slow) constant linear rate, between an initial selected potential difference and a final (maximal) potential difference, and back to the initial potential difference. Detection of current through the working electrode along the varying potential provides indication on electrochemical interaction with the electrode or any material bound thereto, and provides indication on presence and quantity of selected reactants.

FIG. 10B illustrates an exploded view of the electrochemical chip device (10). In this example the measurement chamber (11c) is defined between a base portion (11b) to which the electrodes potion of the substrate (13) is fitted, and a cover portion (11v) configured to sealably attach over the electrodes' potion of the substrate. The cover (11v) comprises a cavity (11c) configured to enclose the electrodes and define the measurement chamber of the chip device (10), and a sample insertion opening (11p). The sample insertion opening may generally be sealably covered by a sealer (11r). The chamber may further comprise one or more filters along general flow of sample material between sample insertion opening (11p) and the measurement chamber (11c), and downstream of the measurement chamber toward optional output port (not specifically shown). The one or more filters may include an inlet filter (11x), generally configured to be a “rough” filter, e.g., having pores in a range between 1 μm and 5 μm, and an outlet filter (11y), generally configured to be a fine filter, e.g., having pores in range between 100 nm and 1000 nm. Generally, the “rough” filter (11x) is configured to separate the electrolyte-containing sample loading chamber from the measurement chamber where large objects, such as cell debris, are filtered out. For example, the rough filter (11x) may have pores with average size of 2 μm. The fine filter (11y) is generally configured to separate the measurement chamber from a reservoir and to filter all objects, organisms, molecules, or any entity, that may be associated with the measurement, thereby maintaining such objects within the measurement chamber (11c). In some examples, filter (11y) may have pores of average size of 500 nm.

FIG. 10C exemplifies insertion of sample into chip device (10) using sample collector (12). In this example, sample collector (12) is configured to perforate the sealer (11c) and introduce bacteria cells from the sampler into the chip device (10). The bacteria cells are transmitted into the measurement chamber (11c) enabling interaction of the bacteria cells with one or more binding sites/moieties on the working electrode (e_{w}).

The electrode array (e_{w},eᵣ,e_{c}) is connectable to an electronic device through respective contact pads (11w,11r,11c) e.g., extending perpendicular to the package, for providing electrical current/voltage and enabling EIS and/or voltametric measurements in the sample. When operating on first chip device, the electronic device is configured to provide voltage signal in selected varying frequencies to determine of impedance between the electrodes. When operating on a second chip device, the electronic device is configured to vary voltage in cyclic, generally slow, way and determine current response along the voltage variation range. To this end, contact pads (11w,11r,11c) may extend outside of chip device (10) enabling inserting of the contact pads end as a ‘dongle-like’ attachment to a selected electronic device for performing measurements. In some configurations, the electronic device is configured to provide potentiostat measurements, typically acting as potentiostat device. The electronic device may be connectable/operated by one or more processors and corresponding computer readable instructions. For example, in some embodiments, the electronic device may be connectable (using wired or wireless connection) to a hand-held electronic device (e.g., a smartphone) carrying computer readable instructions for performing electrochemical impedance spectroscopy (EIS) measurement using the electrode array (e_{w},eᵣ,e_{c}) and provide corresponding readouts. The electronic device may also include a user interface enabling presentation of EIS readout, as well as storage and/or network communication ports for storing the readout data and transmitting such data to remote systems for analyzing. The electronic device may also be responsible for data acquisition and storage e.g., using internal storage and/or remote/cloud storage.

Generally, in some configurations, electrochemical chip device (10) of being second chip device may be configured to receiving input sample formed the is pre-treated by introduction of at least one first binding molecule specific to selected target (e.g., toxin) in the sample, and at least one second binding molecule that is specific to the first binding molecule. The second binding molecule includes or carries at least one labeling moiety that may include and/or produces at least one electroactive product. This enables the use of voltammetry to determine level of generation of the electroactive product, providing indication of binding molecules that attach to the working electrode. The level of electroactive reaction is thus inversely indicative of quantity of the target in the sample. Typically, the sample in the measurement chamber may be allowed to incubate with the electrodes for a selected time, and washed to remove material that is not bound to the working electrode.

Biofunctionalization of the working electrode (e_{w}) can be carried out using thiol chemistry. The mAbs are first thiolated by incubation with Traut's reagent at a molar ratio of 1:15 for 1 hour at room temperature followed by washing with 0.1M phosphate buffer pH 5 to remove the unreacted reagent. Thiolated mAbs are then covalently immobilized onto the gold working electrodes (e_{w}) of the chips devices (10) by drop-casting after thoroughly cleaning the electrodes by immersing 20 min in a solution of 50 mM KOH and 25% H₂O₂ followed by thorough rinsing with Milli-Q water.

FIG. 15 generally describes technique for characterization of sample impedance using EIS technique according to some embodiments of the present disclosure. FIG. 15 illustrates operational actions typically implemented by the electronic circuit connectable to the electrical contacts (13w,13r,13e) of the electrodes in accordance with EIS techniques. As shown, the technique includes applying a voltage probe signal S1, typically in a selected signal frequency, and monitoring current passing through the electrodes in response S2. Based on the amplitude and phase relation between voltage and current the technique include determining cell impedance response S3. This can be visualized using Nyquist plot associated with equivalent electronic circuit S4. The impedance is given by the general notation indicting v=Zi, all being functions of signal frequency. Generally, impedance of the cell depends on interaction between any binding site on the working electrode (e_{w}) and biological materials in the measurement chamber (11c). In accordance with impedance variations, typically visualized by Nyquist plot, the technique includes determining charge transfer resistance S5. Impedance signature, including generally resistance, capacitance, and inductance, i.e., real and imaginary portions of the impedance, provide a signature of cells in the sample S6. Generally, in accordance with the selected binding sites and/or target binding/recognition moieties, carried by the working electrode, this enables determining data on one or more target, for example, bacteria types in the sample based on interaction of the target with the respective binding sites.

FIG. 11 shows a further detailed view of an electrochemical chip device according to some embodiments of the present disclosure. As shown, the electrochemical chip device is generally formed by an electrode arrangement, carrying at least one working electrode and at least one counter electrode, and typically also at least one reference electrode. The measurement chamber may be defined using one or more filters as described above, as well as sample input port. Once a sample collector is placed at the input port, e.g., perforates the seal, the device may utilize a plunger for introducing sample material into the measurement chamber. The plunger is illustrated in FIG. 11 by a syringe, and may be integral to the chip device or connectable thereto. Plunger operation generally pushes liquids through the chamber, extracting bacteria cells from the sampler towards the measurement chamber. Within the measurement chamber, the introduced bacteria may interact with one or more binding sites on the working electrode end located therein. Interaction between the bacteria and the binding sites (e.g., antibody) varies electrical characteristics between the working and counter electrodes, measurable using EIA technique. To this end, the electrode array may be connectable to an electronic device, exemplified in FIG. 11 by a smartphone device carrying a USB stick potentiostat, for providing electrical signals in accordance with EIS technique. The electronic device may also include one or more processors, memory, and communication ports for providing voltage signals, determining current response between the electrodes and determining impedance variation of the circuit as described above. The electronic device thereby provides electrochemical impedance spectroscopy (EIS) readouts, store such results, transmit the results and/or provide further processing.

FIGS. 12A to 12E illustrate the use of mAb-EspB-B7 as binding site in electrochemical chip device as described herein. FIG. 12A illustrates binding of bacterial EPEC cells to mAb-EspB-B7 and respective Nyquist plot; FIG. 12B shows Nyquist plot measurements using bare electrode, electrode carrying mAb-EspB-B7 binding sites and detection in a sample containing purified EspB protein; FIG. 12C shows relative charge transfer resistances (R_{ct}) for samples containing different amounts of charge transfer resistance compared to reference electrodes and samples; FIG. 12D show an exponential fit (using log scale) between detected R_{ct} values and EspB concentration; and FIG. 12E illustrates changes in R_{ct} for specific binding of WT EPEC cells is indicated, resulting in a larger contribution to R_{ct} compared between EPEC WT and ΔespB samples.

FIG. 12A illustrates the details of EIS-based detection of whole bacterial EPEC cells. In this non-limiting example, electrochemical chips as described herein interact with bacterial EPEC cells, thereby varying impedance response along the electrode array. In this example, the electrode array includes a working electrode e_{w} radius of about 0.3 mm, counter electrode e_{c} having radius of about 0.6 mm, and a square reference electrode eᵣ having surface area of about 0.25 mm², and respective contact pads 13w,13c,13r electrically connecting thereto. The working electrode is modified with a thiolated mAb-EspB-B7 using thiol-gold chemistry. The electrodes e_{w},eᵣ,e_{c} are enclosed inside an electrochemical cell structure, configured to receive a sample.

The immobilization of mAb-EspB-B7 and capture of antigen affect the impedance measured between the underlying electrodes as shown in FIGS. 12B to 12E. As shown, an EIS measurement allows for the interrogation of the electrochemical system and separation of the individual components that affect the electrochemical cell circuit established by introducing the sample into the electrochemical cell (cᵢ). The generated Nyquist plot may be fitted to an equivalent circuit from which the different resistance values are extracted (illustrated in an inset in FIG. 12A).

The Nyquist plots shown in FIG. 12B were obtained by EIS measurements of a bare gold working electrode (bare GE), working electrode after the immobilization of mAb-EspB-B7 (GE+mAb) thereon, the mAb-EspB-B7-coated working electrode after incubation with 250 μg/mL purified EspB protein (GE+mAb+EspB). Variation between the Nyquist plots indicates the electrochemical effects of the binding sites and interaction thereof of materials in the sample, thus enabling characterization of the sample.

A suitable one-dimensional parameter that can be extracted from the Nyquist plots, using equivalent circuit fitting, in the relative charge transfer resistance (Rea) values. FIG. 12C shows measured R_{ct} values for different concentrations of purified EspB protein (1, 4, 10 and 250 μg/ml), reference sample using modified working electrode. This variation demonstrates a dose-dependent increase in the detected R_{ct} values. Relative R_{ct} values are the means of the R_{ct} ratios (before and after antigen capture) calculated from 3-6 measurements. Error bars represent the ±SD. The variation in R_{ct} was fitted to exponential formula as a function of EspB protein concentration as shown in FIG. 12D. This model provides a fit R² of 0.978 indicating good agreement with the results.

FIG. 12E shows measurement of specific binding of WT EPEC cells. The specific binding is indicated by larger contribution to R_{ct} compared with the ΔespB null strain. The percent change in R_{ct} ratios measured for EPEC WT and ΔespB was calculated and averaged from 20 repeating measurements (five measurements each containing four samples) for each strain.

FIG. 13 schematically illustrates an electrochemical cell device (cᵢ) using electrode array and electronic circuit for EIS measurement. The working electrode carried binding sites formed of the mAb-EspB-B7 to provide selective binding to EPEC cells. This is illustrated in FIG. 13 as E-coli cells do not attach to the binding sites and therefor provide EIS measurement associated with working electrode coated by the mAb-EspB-B7 binding sites that do not interact with bacterial cells. Presence of EPEC cells result in suitable interaction varying the EIS results as shown in FIG. 12C.

FIG. 14 shows an arrangement of electrode arrays on a chip device (PCB) and modification of the working electrode with selected binding sites. The working electrode may be formed of gold, or any other suitable electrically conducting metal, carbon, or conductive polymeric material that can be used as a working electrode in an electrochemical setup. The working electrode is coated by anti-pathogenic E. coli monoclonal antibodies such as: anti-EspB or others specific mAb's. Inset image of FIG. 14 shows impedance response measured over a predefined frequency range, according to some embodiments. The impedance spectra is fitted to an electric circuit (right) to determine simplified parameter such as charge transfer resistance R_{ct}. Specific binding of selected antigens affects certain circuit parameters and enable detection and quantification of the antigen bound hereto.

FIGS. 16A to 16D illustrate various configuration of electrochemical cells chip devices and electrode arrangement thereof. FIG. 16A shows chip configuration having a plurality of electrochemical cells; FIG. 16B illustrates an arrangement of a plurality of working electrodes in a single electrochemical cell; FIGS. 16C and 16D illustrate components in exploded and assembled views.

As shown in FIG. 16A, the device 60 may be formed as a printed circuit (e.g., chip) including a plurality of individual electrochemical cells (C₁, C₂, . . . Cₙ). Each electrochemical cells (Cᵢ) includes at least working (e_{w}) and counter (e_{c}) electrodes and is shows to also include a reference electrode (Eᵣ). Generally, electrode arrangement of each cell is associated with respective electronic circuit represented by respective potentiostat circuitries (65) for applying EIS measurement technique therethrough. The different electrochemical cells (C₁, C₂, . . . Cₙ) may be placed within a common measurement chamber, where each cell carries different binding sites, or configured to be placed in separated measurement chambers to simultaneous analysis of different samples.

Thus, the multiple electrochemical cell arrays (C₁, C₂, . . . , Cₙ) shown in FIG. 16A are formed on a common substrate (13), where each electrochemical cell (Cᵢ, where 0≤i≤n is an integer) includes individual working, reference and counter electrodes (e_{w},eᵣ,e_{c}). In such embodiments the “reader” circuitry can be implemented utilizing respective potentiostat circuitries (65) for each one of the electrochemical cells. The measurement data generated by the potentiostat circuitries (65) may be used in various processing technique. For example, the measurement data may be digitized by digitizer unit (60a) for processing using a processing unit (60u) to determine amounts of bacteria suspension over the mAbs coated working electrodes (e_{w}) in the different electrochemical cells. The determined results can be locally stored in the memory device (60m), and/or communicated (wirelessly or over data lines) to external system/device (not shown) by the interface unit (60i).

Generally, device 60 may be formed of an arrangement of first chip devices and second chip devices in accordance with bind sites of the working electrodes in each electrochemical cell array, voltage signals applied between the electrodes and data processing thereof. As described above, treatment of the sample for the second chip devices may also include introducing additional binding molecules and labeling moiety, as the working electrode is generally connected to the target or parts thereof. The binding molecules generally include at least one first binding molecule that is specific for the at least one target, and at least one second binding molecule that is specific for the first binding molecule. The labeling moiety attached to the second binding molecules may be used to promote certain electrochemical reaction detectable by voltammetry.

The chip configuration (69) illustrated in FIG. 16B utilizes a plurality of working electrodes (e₁, e₂, . . . eₙ) associated with a single electrochemical cells (cᵢ). the different working electrodes may carry respective one or more different binding sites and may be operated using a common electronic circuit (e.g., single potentiostat circuitry) (65), or using one or more different electronic circuits. When operating using a common circuit, the readout may be enables using a multiplexer device (60x) providing selective signal feed to the different working electrodes (e₁, e₂, . . . eₙ), enabling to differentiate between readout from the different electrodes. Generally, the multiplexer device (60x) may also include circuitry, or connected to a control circuit, configured for varying profile of electrical signal provided by the potentiostat, when feeding a first chip device or a second chip device.

Similarly to FIG. 16A output EIS signals may be digitized (60a) and transmitted for processing by processor (60u) to provide indication of one or more bacteria in the sample. This configuration enables (multiplexed) sequential measurements of a sample for various different agents (different bacterial agents).

FIGS. 16C and 16D illustrate another chip configuration (69) including a plurality of working electrodes (e_{w}) a respective plurality of reference electrodes (eᵣ) and a common counter electrode (e_{c}). As seen, in this non-limiting example each reference electrode (eᵣ) is positioned adjacent its respective working electrodes (e_{w}), and the common counter electrode is positioned around the arrangement of the plurality of working electrodes (e_{w}) and reference electrode (eᵣ). The different working electrodes maybe modified to carry similar or different binding sites in accordance with desired sample analysis profile.

The substrate (13) carrying the electrodes may be any insulating substrate. Generally, the respective electronic EIS circuitry (e.g., potentiostat circuitry) may be placed on the same substrate as the electrodes, or connectable thereto via contact pads. Accordingly, this configuration may be implemented as a printed circuit boards, foils or film on which the electrode arrangement is deposited. In some embodiments the substrate may be fabricated using a semiconductor (e.g., Silicon) substrate and conventional semiconductor production techniques to implement the circuitries and electrodes on/in the substrate.

As indicated above, the present disclosure also provides a biosensor system for MC-LR detection. FIGS. 17A to 17D illustrate scheme of operation and fabrication of the biosensor system. FIG. 17A is a schematic illustration of the biochip sensor and includes an illustration of sensor operation. The sensor includes an electrode arrangement as described above. The working electrode is connected directly or indirectly to Anti-MC-LR monoclonal antibodies. Generally, the Anti-MC-LR monoclonal antibodies may be chemically modified and covalently immobilized to the working electrode surface. The electrode arrangement is connectable to an electronic device (e.g., potentiostat illustrated by generalized circuit diagram). When the active ends of the electrode arrangement is exposed to a sample contaminated with MC-LR-secreting cyanobacteria, the toxins bind the electrode-bound antibodies, affecting the electrode's impedance. This change can be measured and analyzed in real-time, allowing the quantification of toxins in the sample.

FIG. 17B shows an EIS measurement results in the form of Nyquist plot. Generally, the impedance data may be processed in accordance with model circuit to determine data on charge transfer resistance Rₛ. The charge transfer resistance is typically indicative of one or more parameters of existence and amount of MC-LR toxins attached to the working electrode and according to amount/concentration in the sample.

FIG. 17C illustrates fabrication process of the electrode arrangement on a substrate. In the fabrication process, (a) a wafer is cleaned with acetone, isopropanol, and distilled water; (b) photoresist (PR) coat is spun onto the wafer and soft baked. (c) Patterns are projected onto the wafer (photolithography); (d) The substrate is developed and unexposed PR is removed. (e) Titanium and gold are sputtered onto the substrate (f) The PR and gold are removed by a lift-off process. Following this, the wafer is rinsed with ACT, IPA, and DI, and (g) The wafer is ready for electroplating. FIG. 17D shows surface characterization of the deposited electrodes. In this specific figure, the reference electrode is shown. Generally, the reference electrode provides potential reference and should not carry any current to or from the sample. Accordingly the reference electrode may be formed by electroplating silver (from a silver plating bath) followed by anodic generation of a silver chloride layer to obtain a silver/silver chloride layer (Ag/AgCl).

A configuration of the complete biosensor system is illustrated in FIGS. 18A and 18B. FIG. 18A shows an electrode arrangement providing 8 biosensor chip devices fabricated on substrate. The electrode arrangement is configured to be positioned within a casing, defining a plurality of measurement chambers, where a set of three electrodes is positioned within each measurement chamber. FIG. 18B shows custom manufactured system including an arrangement of biochip devices. Electrode arrangement of each measurement unit are separately connectable to electrode device enabling selective electrochemical detection process. More specifically, in accordance with material selection connected to the working electrode, the different chip devices may utilize EIS, voltammetry or amperometry as described herein. FIG. 19 and FIGS. 20A to 20C show characterization of the chip device operating as electrochemical cell. FIG. 19 shows electrical verification of an Ag/AgCl reference electrode, carried out by measuring its potential versus a commercial reference electrode in varying electrolyte (NaCl) concentrations indicating the electrode demonstrates a ‘Nernstian behavior’, close to the theoretical value.

FIG. 20A-20C show verification measurements of the complete biosensor chip by voltametric techniques. FIG. 20A shows cyclic voltammogram at scan rates of 50 mV/sec, 100 mV/sec, 150 mV/sec and 200 mV/sec. The voltammogram is performed with a solution of 20 mM ferricyanide/ferrocyanide. FIG. 20B shows peak current analysis for anodic (top) and cathodic (bottom) currents through the voltametric characterization showing increased peak height linear with square root of the scan rate. FIG. 20C shows that the peak separation is generally independent of the scan rate.

FIG. 21A-21B illustrate biofunctionalization of the working electrode. FIG. 21A shows antibody modification and immobilization using covalent attachment. Following the biofunctionalization, FIG. 22A-22D show surface characterization of functionalized electrodes. As shown, the immobilized antibodies are shown in AFM image in FIG. 22E-22F as compared to bare gold electrode.

Activity of the working electrode is illustrated in FIG. 23 shows Nyquist plot detected by EIS analysis of the system using base working electrode (bare GE), working electrode modified by immobilized antibodies on clean solution (GE+mAb), and modified electrode following binding with toxins from solution at 3 μg/L concentration (‘3 μg/L’). As shown, absolute value of the impedance is increased with attachment of the antibodies to the electrode, and further with binding of the toxins, indicating increase in charge transfer resistance Rct. This increase is proportional to the concentration of the bound toxin and allows its quantification in the sample.

FIG. 24 shows a comparison of charge transfer resistance for different incubation times. MC-LR in a sample was allowed to bind to MC10E7/GE at incubations times of 10 minutes, 30 minutes, and 60 minutes. The measurements were conducted in PBS pH 7.4 containing 10 mM Fe(CN)₆^{4-/3-} and 0.1 M KCl and show variation in charge transfer resistance (R_{ct}) on immunoreaction time. Bar plots (change in R_{ct} response) were calculated from the ratio of MC-LR/MC10E7/GE and MC10E7/GE normalized to 1 (error bars: SEM, n=3).

Concentration measurements on various solutions with different toxin levels are shown in FIGS. 25A to 25D. FIG. 25A shows Nyquist plots obtained by EIS measurements of a bare gold electrode (‘bare GE’), electrode after the immobilization of anti-MC-LR mAb (‘GE+mAb’), and after incubating with six different concentrations of purified MC-LR toxin: 0.0003, 0.003, 0.03, 0.3, 3, and 30 μg/L. (The lowest concentration yielded a similar impedimetric signal as the background). The increase in charge transfer resistance is shown with increase of absolute value of the impedance. FIG. 25B shows charge transfer resistance values of purified MC-LR toxin protein for varying toxin concentrations, showing increase in resistance with concentration. The increase shows exponential rise in Rct with concentration as illustrated in FIG. 25C. This provides a calibration curve for target MC-LR, enabling to determine concentration in an unknown sample.

FIG. 25D charge transfer resistance variation in charge transfer resistance for toxins obtained from different solutions. As shown specific binding of MC-LR, contributes to an increase in R_{ct} in Microcystis suspensions, whereas no response was observed with Spirulina suspensions. Higher signals were obtained from filtered Microcystis suspension, as expected. Incubation of MC-LR on an electrode functionalized with an unrelated antibody (mAb-EspB-B7), showed no MC-LR binding, further supporting the specificity of the biosensor. The changes in R_{ct} values (% ΔR_{ct}) are the means of the Ret ratios (before and after antigen-capture), calculated from triplicates.

As indicated above, the present disclosure also provides for detection of toxins using voltametric measurement based on competitive ic-ELISA. FIG. 26A-26B illustrate measurement results for ic-ELISA for Microcystin-LR detection. Different concentrations of MC-LR were detected by ic-ELISA ranging from 0.03 μg/L to 30 μg/L in FIG. 26A. FIG. 26B show standard curve obtained from ic-ELISA measured in 8 repeats of ELISA plate wells that were coated with 3 μg/mL MC-LR toxin. The antibody MC10E7 dilution was 1:3,000; enzyme Immunoconjugate dilution was 1:4,000. The experimental data are shown as a discrete plot with error bars in black. The solid black curve is a fit of the Hill equation to the experimental data using OriginLab. The inset image shows the range of quantitative detection with good linearity.

As indicated above, the present disclosure utilizes electrochemical ELISA measurement technique utilizing voltammetry and/or amperometry analysis using a working electrode, where the working electrode is connected directly or indirectly to the target (e.g., toxin) or any component thereof. First antibody specific to the toxin and second antibody specific to the first antibody, and carrying a detectable label are added to the sample. According voltametric and/or amperometry analysis provides output data on level of antibodies bound to the electrode, which is generally inverse to concentration of the toxin in the sample.

FIGS. 27A-27B show raw cyanobacterial cultures used as a model for contaminated water.

FIG. 27A figure shows whole bacterial cell suspensions of Microcystis aeruginosa PPC 7806, and FIG. 27B shows whole bacterial cell suspensions of Spirulina sp. Both samples were cultured, grown, and maintained in BG-11 at a temperature of 24-26° C. and light intensity of 6 μmol photons m⁻²s⁻¹. It should be noted that the present technique may generally utilize electrical characteristics of the sample for determining data on toxins in the sample.

FIGS. 28A-28B illustrate assessment of the specificity of target binding site and its effectiveness in determining target in the sample. FIG. 28A shows obtained Nyquist plots from measurements of a bare gold electrode (‘bare GE’), electrode after the immobilization of mAb-EspB-B7 (‘GE+mAb-EspB-B7’), and after incubation with 2 μg/mL purified MC-LR toxin. As shown, the impedance response varies due to presence of the MC-LR on the electrode functionalized. In FIG. 28B, the electrode is modified with a nonspecific antibody (mAb-EspB-B7). Accordingly, presence of MC-LR in the sample is not visible in the impedance measurement, indicating no MC-LR binding.

To provide detection of the target in the sample, the present disclosure utilizes first and second binding molecules. More specifically, the technique may utilize treating the sample to further include at least one first binding molecule that is specific for the target, and at least one second binding molecule that is specific for the first binding molecule. Also, the said second binding molecule carries, or is connected to, at least one labeling moiety that comprises and/or produces at least one electroactive product. FIG. 29 is a schematic illustration of the resulting electroactive reaction, i.e., indirect competitive ELISA. The first binding molecules include antibodies (mAbs) that are specific to MCs. The antibodies may be incubated with the antigen to be measured in the raw sample. This forms antigen-antibody (Ag-Ab) complexes with free and unbound mAbs. The sample is added to a well plate-coated MC-LR toxin, exemplifying the working electrode of the second chip device, and the free mAbs bind to the adsorbed MC-LR on the plate well. The combine chip device may generally be incubated and washed, to maintain bound material. And HRP-conjugated secondary antibody is added followed by a substrate. This allows enzymatic electro-active reaction of the substrate. In typical ELISA, the reaction produces a color that can be measured using an ELISA plate reader. The present disclosure utilizes a working electrode as the plate-coated MC-LR toxin, enabling electrochemical measurement of the labeling interaction, e.g., the HRP causing substrate reaction.

FIG. 30A-30E illustrate binding target or a portion thereof on the working electrode and electrochemical detection of the target in a sample according to some embodiments of the present disclosure. FIG. 30A shows Mercaptoundecanoic acid (MUA) modified gold surface of the working electrode of the second chip device. Moving to FIG. 30B EDC/NHS activates the MUA gold surface to enable binding to the target. In FIG. 30C target molecules, in this example MC-LR, are immobilized on an activated gold working electrode. At this stage the working electrode is ready to operate. The electrode in the chip device is interacted with a sample for detection of presence and quantity of the target (e.g., MC-LR) in the sample. As mentioned above, selected binding molecules (e.g., antibodies) are added to the sample. This is exemplified by BSA and HRP-Ab conjugate illustrated in FIG. 30D. The binding molecules attach to the target in the sample, as well as to the target (or portion thereof) bound to the working electrode. The sample may be washed away from the measurement unit, leaving only material that is bound to the electrode to remain. In FIG. 30E substrate material, e.g., 1°-HRP-Ab conjugate complexes, is added to the measurement chamber, to interact with the labeling moieties on the MC-LR-coated working electrode. Unbound antibodies are removed by washing, and the level of binding molecules attached to the working electrode inversely relate to quantity og the target in the original sample. The voltametric measurement may range a few hundreds of mV to allow the electrochemical interaction to take place and be detected, FIG. 31A-31C exemplify target detection using optical ELISA technique. FIG. 31A shows absorbance for different concentrations of MC-LR ranging from 0.03 μg/L to 30 μg/L (error bars: SD, n=3). The theoretical/standard curve of ic-ELISA was measured in 8 repeats of ELISA plate wells that were coated with 3 μg/mL MC-LR toxin. The antibody MC10E7 dilution was 1:3 000; enzyme Immunoconjugate dilution was 1:4 000. FIG. 31B shows the absorbance results in a plot with error bars in black. The solid black curve is a fit of the Hill equation to the experimental data. FIG. 31C shows a range of quantitative detection with good linearity.

FIG. 32 exemplifies voltammogram measured on sample containing 8 mM Fe(CN)₆^{4-/3-} and samples containing four different concentrations of MC-LR solutions (20 and 30 μg/L) in PBS (pH 7.4) at a scan rate of 100 mV/sec. As shown, the MC-LR by itself is not electrochemically active. This allows MC-LR coated working electrode to operate efficiently in voltametric analysis of a sample to determine quantity of MC-LR therein.

FIG. 33A-33B exemplify measured cyclic voltammetry at a scan rate of 50 mV/sec of PBS, pH 7.4, substrate (a mixture of 0.3 mM H₂O₂, and 0.45 mM APAP), and the reaction of HRP with the substrate. FIG. 33A shows results of a scan initiated following 1 min incubation of solution reactants. CVs were performed separately, and FIG. 33B shows repeated CV cycles at a scan rate of 50 mV/sec of a solution containing 0.3 mM APAP and H₂O₂ and 0.5 μg/ml HRP. FIG. 33A clearly shows electrochemical raction detectable in the sample containing both substrate and HRP, while no reactions in other samples. FIG. 33B exemplifies diminishing of the reaction along time. The scans in FIG. 33B where initiated following 1 min incubation of solution reactants and cycles 4-6 were initiated after a 2 minutes pause, where no potential was applied.

Thus, as exemplified by the above figures, a working electrode connected to molecules of the target as described herein may be used for voltamteric detection of the respective target in a sample using selected binding molecules in the sample.

FIG. 34A-34B exemplify characterization of the electrode. FIG. 34A shows Nyquist plots of the electrode and FIG. 34B shows analysis using an equivalent Randles circuit. The electrodes used include bare gold electrode (ge), the EDC-NHS/MUA/ge functionalized electrode without MC-LR target, and electrode carrying immobilized MC-LR toxin. The characterization was performed in the presence of 10 mM Fe(CN)₆^{3-/4-} in 1×PBS (pH 7.4). Impedance spectra were acquired at the formal potential of 10 mV in the 10 kHz to 0.1 Hz frequency range. FIGS. 34A and 34B exemplify increase in charge transfer resistance with the larger elements attached to the working electrode. This measurement is in agreement with the EIS detection technique described herein, and further provides indication to presence of target molecules (or at least portions thereof) connected to the working electrode.

Accordingly, the present technique may utilize one or more, or combination, of electrochemical chip devices using EIS analysis and/or voltammetry analysis. The different chip devices utilize working electrode connected directly or indirectly to selected binding sites. In the first chip device, the binding site may include binding molecules specific for binding of the target sought. In the second chip device, utilizing voltametric analysis, the binding site may include the target or a portion thereof, and the sample may be treated to enable competitive ELISA technique, within the electrochemical chip device, and determine electrochemical data thereof.

Accordingly, in some embodiments, the present disclosure provides a biosensor chip carrying an electrode arrangement formed of at least two electrodes comprising at least one working electrode carrying at least one target binding site and/or moiety, and at least one counter electrode. The biosensor chip may be configured to place the electrodes within a measurement chamber to be in liquid communication with sample solution, for analysis of one or more agents within the sample solution that attach to the at least one target binding site and/or moiety. The biosensor chip is connectable to an electronic device for electrical analysis of impedance between the electrodes, thereby determining data on the one or more agents within the sample solution.

Generally, the EIS analysis describe above may refer to faradic current transmitted between the working and counter electrodes, passing through the sample solution. This current may vary in response with attachment of one or more agents within the sample solution to the working electrode, thereby adjusting charge transmission into the sample solution.

In some embodiments, the plurality of electrodes of the biosensor chip device of the present disclosure may comprise at least one working electrode, at least one counter electrode configured to introduce electrical currents into the measurement chamber, and at least one reference electrode for measuring electrical voltage between the at least one working electrode and the at least one reference electrode. In more specific embodiments, the at least one working electrode is connected directly or indirectly to at least one target binding site and/or moiety. The reference electrode may provide reference impedance data associated with electrical characteristics of the sample solution, while being generally invariant to the one or more agents within the sample solution that attach to the at least one target binding site and/or moiety of the working electrode.

In yet some further embodiments, the biosensor chip device of the present disclosure may further comprise at least one inlet for introducing the sample into the measurement chamber; and at least one inlet filter for selectively passing the sample from the inlet into said measurement chamber.

In some other embodiments, the chip device of the present disclosure may comprise an outlet formed in the packaging assembly and at least one outlet filter for selectively passing sample material from the measurement chamber to the outlet.

Still further, in some embodiments, the packaging assembly of the biosensor chip device of the present disclosure comprises a base portion configured to receive the electrodes portion of the substrate, and a cover portion having an open cavity and configured to sealably attach to the base portion over the electrodes portion of the substrate and define the measurement chamber by its open cavity.

In some embodiments, the chip device of the present disclosure may comprise a plurality of measurement chambers, each comprising at least three of the plurality of electrodes defining a working electrode, a reference electrode, and a counter electrode, and a respective plurality of potentiostat circuitries each of which electrically connected to the at least three electrodes of its respective measurement chamber. More specifically, when referring to a plurality of measurement chambers and/o to a plurality of electrodes and/or a plurality of potentiostat circuitries, it is meant that in some embodiments, at least 3, 6, 9, 12, 15, 18, 21, 24, 27, 30 or more, 60, 90, 120, 150, 180, 210, 240, 270, 300 or more.

In some embodiments, the plurality of electrodes in the measurement chamber comprises define a plurality of working electrodes, at least one reference electrode, and at least one counter electrode. Still further, the device may comprise a single electronic circuit (e.g., potentiostat circuitry) and a multiplexer device configured to selective transfer signals measured by the plurality of working electrodes to the single potentiostat circuitry.

In some embodiments, Biosensor measurements are based on Electrochemical Impedance Spectroscopy (EIS). The faradaic current response of a routinely employed redox couple (10 mM K₃Fe(CN)₆) found within the measurement buffer, is monitored by EIS. The impedance spectra are obtained with a potential amplitude of 5 mV at a frequency range between 100 kHz and 10 Hz. The charge transfer resistance (R_{ct}) values may be obtained by fitting the generated Nyquist plots to equivalent circuits. The percent change in charge transmission resistance R-ratios between the biofunctionalized electrodes and varying EspB concentrations may be determined in accordance with ΣR_{ct}(EspB)/R_{ct}(mAb)−1.

In some embodiments of the device of the present disclosure, a variety of different types of working electrode may be used. For example, the working electrode may be carbon electrode, including glassy carbon, activated carbon cloth electrode, carbon felt, platinized carbon cloth, plain carbon cloth etc. the working electrode may be made of any conductive metal, for example, gold, platinum or silver, or any other conductive material including polymeric materials. The counter electrode may be made of similar material as the working electrode, or of a selected different conductive material. The reference electrode may for example be saturated calomel electrode, may be an Ag/AgCl electrode. Furthermore, the electrodes may be of a screen-printed electrode which can be inserted into the vessel comprising the cells without the need to withdraw a sample and transport it into a separate electrochemical cell. The electrodes used in the device of the invention, to detect the target according to the methods of the present disclosure, may be reusable electrodes or disposable ones. Reusable electrodes may for example be electrodes made of glassy carbon in a disk or rod shape which are embedded in Teflon. Disposable electrodes may for-example be electrodes in the form of carbon paper, carbon cloth, carbon felts, or the screen-printed electrode of the kind noted above. According to some embodiments, the electrochemical cell is a three-electrode cell. According to other embodiments, the electrochemical cell is a two-electrode cell. According to some further embodiments, the electrochemical cells are provided as an array (i.e. chip) comprising a plurality of such cells i.e. a multi-well/multi-spot array where each well is of a nano-volume size.

Still further, in some embodiments, the biosensor chip system of the present disclosure may further comprise at least one inlet for introducing the sample into the measurement chamber; and at least one inlet filter for selectively passing the sample from the inlet into the measurement chamber.

In some embodiments, the biosensor chip system of the present disclosure comprises an outlet formed in the packaging assembly and at least one outlet filter for selectively passing sample material from the measurement chamber to the outlet.

In certain embodiments of the biosensor chip system of the preset disclosure, the packaging assembly comprises a base portion configured to receive the electrodes portion of the substrate, and a cover portion having an open cavity and configured to sealably attach to the base portion over the electrodes portion of the substrate and define the measurement chamber by its open cavity.

In some embodiments of the biosensor chip system of the present disclosure, the first and second plurality of electrodes of at least one of the first and second chip devices comprises at least one working electrode, at least one counter electrode configured to vary electrical potential and enable current transmission into the measurement chamber, and at least one reference electrode for measuring electrical voltage between the at least one working electrode and the at least one reference electrode.

Still further, in some embodiments of the biosensor chip system, the at least one electronic device comprises one or more potentiostat circuitries connected at the one of first and second chip devices.

In some further embodiments, of the biosensor chip system according to the invention, the at least one electronic device comprises a plurality of potentiostat circuitries. More specifically, the system comprising a plurality of measurement chambers comprising at least one first measurement chamber associated with the first chip device and/or at least one second measurement chamber associated with the second chip device. Each of the measurement chambers comprises at least three of the plurality of electrodes defining a working electrode, a reference electrode, and a counter electrode, and is associated with respective potentiostat circuitries electrically connected to the at least three electrodes of its respective measurement chamber.

In some embodiments of the biosensor chip system of the present disclosure, the plurality of potentiostat circuitries comprises at least one first potentiostat circuitry associated with electrodes of the first chip device and configured for operating electrochemical impedance spectroscopy (EIS), and at least one second potentiostat circuitry associated with electrodes of the second chip device and configured for operating at least one of voltammetry and amperometry measurement.

In yet some further embodiments, the biosensor chip system of the present disclosure comprises a plurality of one or more first chip devices and one or more second chip devices located in a plurality of separated measurement chambers, the respective pluralities of electrodes comprise a plurality of working electrodes, reference electrode, and counter electrodes. It should be noted that the device comprises a potentiostat circuitry and a multiplexer device configured to selective transfer signals between the respective pluralities of electrode to the potentiostat circuitry.

As noted above, the biosensor chip system in accordance with the preset disclosure is particularly usable for identifying and/or quantifying and/or monitoring at least one target in a sample. In some embodiments, such target may be at least one small molecule compound.

A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In more specific embodiments, the biosensor chip system of the present disclosure may be particularly useful for detecting small molecule compounds that may comprise at least one toxin. Non-restrictive samples of these include veterinary residues, small molecules as well as metal ions, drugs of abuse, toxins, pesticides, personal care products (including human pharmaceuticals, fragrances, etc.) and industrial.

In yet some further embodiments, such at least one toxin, may be any toxin produced by at least one pathogenic organism. In yet some further particular and non limiting embodiments, such toxin may be any toxin produced by at least one bacterial cell. In some specific embodiments, the biosensor chip system of the present disclosure may be particularly applicable for detecting, monitoring and/or quantitating at least one toxin, specifically small molecule toxin produced by cyanobacteria. Cyanobacteria, also known as Cyanophyta, Also known as blue-green algae, are a phylum of Gram-negative bacteria that obtain energy via photosynthesis. Blue-green algae play an important role in carbon and nitrogen balance in the biosphere. They produce a high number of bioactive molecules, and certain species produce cyanotoxins that contribute as defense mechanisms against different ambient stress factors. Cyanobacteria are proved in various habitats, such as drinking water reservoirs and recreational waters, at the basis of food chains, and thus, with a substantial impact on ecosystems and human health. Centurial observations of a correlation between water blooms and health issues in animals and humans are extended in numerous epidemiological, in vivo and in vitro, studies. Various bioactive compounds under the common name cyanotoxins are established as the reason for blooming water toxicity.

Thus, in some specific embodiments, the biosensor chip system disclosed herein is applicable for detecting, monitoring and/or quantitating at least one toxin that may be at least one cyanotoxin.

Still further, in some embodiments, the biosensor chip system disclosed herein is applicable for detecting, monitoring and/or quantitating any cyanotoxin, for example, any cyanotoxin of any group, specifically, at least one of: at least one cyclic peptide, at least one alkaloid and at least one lipopolysaccharide, or any combinations thereof.

Still further, the cyanotoxin detectable by the biosensor chip system of the present disclosure is at least one cyclic peptide. In yet some further embodiments, the cyclic peptide is at least one microcystin (MC), and at least one nodularin (NOD).

In some embodiments, the biosensor chip system of the present disclosure is applicable for detecting, monitoring and/or quantitating at least one toxin that is at least one microcystin. More specifically, in some embodiments, the microcystin is at least one of Microcystin-leucine-arginine (MC-LR), Microcystin-arginine-arginine (MC-RR), Microcystin-tyrosine-arginine (MC-YR), and Microcystin-leucine-alanine (MC-LA), and any combination, derivatives and variants thereof.

More than 90 microcystin isoforms, Cyanotoxins have various chemical structures; thus, their toxic effects are due to different mechanisms. Cyanotoxins are classified into three major groups according to their chemical structure: alkaloids (cylindrospermopsin, saxitoxin, lyngbyatoxin-a, and aplysiatoxin,), cyclic peptides (microcystins, MCs, and nodularins, NODs), and lipopolysaccharides. The cyclic pentapeptide nodularins and cyclic heptapeptide microcystins are the most widespread cyanotoxins in water blooms. MCs are produced by different cyanobacterial species (Microcystis, Oscillatoria, Aphanocapsa, Cyanobium, Arthrospira, Limnothrix, Phormidium, Hapalosiphon, Anabaenopsis, Nostoc, and Synechocystis). It is known that nodularins are produced only by cyanobacteria from the genus Nodularia (Nodularia spumigena). Approximately 250 variants of MCs known, with the most toxic and widely distributed MC being MC-LR. NODs and MCs are among the most common natural cyanotoxins. Their toxicity is mainly due to the ability to inhibit the eukaryotic protein serine/threonine phosphatase families 1 and 2A (PP1 and PP2A), which are essential for many signal transduction pathways of eukaryotic cells. This inhibition is linked to protein hyperphosphorylation, thus leading to modification of cytoskeleton and disturbances of many cellular processes: loss of cell-cell adhesion at the desmosomes, disruption of actin filaments, and altered cell signaling pathways, for example MAPKs signaling pathways that regulate cellular proliferation. As potent inhibitors of protein serine/threonine phosphatase, MCs and NODs have a profound effect on cell signaling leading to the affected cell's death.

Microcystins are composed of seven amino acids that include a unique β-amino acid (ADDA). It contains alanine (D-ala), D-β-methyl-isoaspartate (D-β-Me-isoAsp), and glutamic acid (D-glu). Furthermore, microcystins contain two variable residues (positions 2 and 4), which make the differentiation between variants of microcystins. These two variable elements are always standard L-amino acids. In microcystin-LR these are leucine and arginine. These modifications include demethylation of Masp and Mdha and methylesterification of D-Glu. Different microcystins have different toxicity profiles, with microcystin-LR found to be the most toxic. The principal amino acids sequence of microcystins is: cyclo-(d-Ala¹-X²-d-MeAsp³-X⁴-Adda⁵-d-Glu⁶-MDha⁷), where d-MeAsp is d-erythro-β-methylaspartic acid, and Mdha is N-methyldehydroalanine. In some embodiments, the general sequence of microcystins is denoted by SEQ ID NO: 50. In some specific embodiments, the biosensor chip system according to the present disclosure is applicable for detecting, monitoring and/or quantitating at least one microcystin, specifically, Microcystin-LR (MC-LR), or any derivatives and variants thereof. Thus, the invention provides at least one biosensor chip system specifically applicable for detecting, monitoring and/or quantitating MC-LR. MC-LR has leucine in position 2 and arginine in position 4. Specifically, cyclo-(d-Ala¹-Leu²-d-MeAsp³-Arg⁴-Adda⁵-d-Glu⁶-MDha⁷). In some specific embodiments, the amino acid sequence of MC-LR is as denoted by SEQ ID NO: 51.

In yet some further embodiments of the biosensor chip system of the present disclosure, at least one of: (i) the at least one working electrode of the first chip device is connected directly or indirectly to at least one antibody that specifically binds the at least one cyanotoxin; and (ii) the at least one working electrode of the second chip device is connected directly or indirectly to the at least one cyanotoxin. Thus, the disclosed system may comprise working electrode/s that ae connected to antibody that recognizes the specific cyanotoxin, and operates via EIS, or alternatively, and/or additionally, working electrodes that are bound to cyanotoxin itself, and are operated via voltammetry or amperometry, and work in some embodiments in a competitive assay as discussed herein after.

In some embodiments, the biosensor chip system of the present disclosure is applicable for any sample, specifically, any of the samples disclosed by the present disclosure. In some embodiments, the sample is an environmental sample or a biological sample.
a sample can be obtained from food, beverage product, medical devices and surfaces.

A further aspect of the present disclosure relates to a kit comprising:

In component (a), at least one biosensor chip system usable for identifying and/or quantifying and/or monitoring at least one target in a sample. The system of the disclosed kit comprises at least one of: at least one first and at least one second chip devices. Thus, in some embodiments, the kit may comprise a system comprising at least one of the first device, in yet some alternative embodiments the kit disclosed herein may comprise at least one second device. In yet some alternative embodiments, the disclosed kits may comprise at least one system comprising at least one first device and at least one second device. In more specific embodiments, the first chip device of the system of the disclosed kit comprises a first plurality of electrodes connectable to at least one electronic device. At least one of the electrodes is a working electrode. The working electrode is connected directly or indirectly to at least one target binding site and/or moiety. It should be noted that the target binding site and/or moiety specifically binds the at least one target or any component thereof. In some embodiments, the plurality of electrodes is configured for electrochemical impedance spectroscopy (EIS) analysis of said sample. Still further, the second chip device comprises: a second plurality of electrodes connectable to at least one electronic device. The at least one of the electrodes is a working electrode. More specifically, the working electrode is connected directly or indirectly to the target or any component thereof. In yet some further embodiments, the plurality of electrodes is configured for electrochemical voltammetry or amperometry analysis of the sample. Still further, in some embodiments, the kit may further comprises at least one of: (b), at least one control sample and/or control standard value; and (c), instructions for use.

In some embodiments, the at least one biosensor chip system of the disclosed kit is as defined by the present disclosure.

A further aspect of the present disclosure relates to a method for identifying and/or quantifying and/or monitoring at least one target in a sample. More specifically, the method comprising at least one of the following steps: (a) performing an electrochemical impedance spectroscopy (EIS) analysis of the sample, and/or (b), performing an electrochemical voltammetry or amperometry analysis of the sample. More specifically, the disclosed method may comprise the step of performing an electrochemical impedance spectroscopy (EIS) analysis of the sample. In some embodiments, performing such analysis comprises: in a first step (i), contacting with the sample a first plurality of electrodes comprising at least one working electrode and at least one reference electrode, or any first chip device or system comprising the same. It should be noted that the at least one working electrode is connected directly or indirectly to at least one target binding site and/or moiety. The next step (ii), involves applying voltage signal between the at least one working electrode and the at least one reference electrode, and determining electrical current between the electrodes in response to the voltage signals for a selected number of one or more signal frequencies; and (iii), determining relations between electrical current response and voltage signal for the one or more signal frequencies; and determining electrical impedance between the at least one working electrode and the at least one counter electrode. It should be noted that the impedance variation being indicative of presence and/or quantity of the at least one target in the sample.

As discussed herein, the methods of the present disclosure may comprise either as an alternative step, or as an additional step, (b), performing an electrochemical voltammetry or amperometry analysis of the sample. In some embodiments, such additional and/or alternative analysis comprising: (i), contacting with the sample a second plurality of electrodes comprising at least one working electrode and at least one reference electrode, or any second chip device or system comprising the same. In some embodiments, at least one working electrode is connected directly or indirectly to the at least one target or any component thereof. Accordingly, the sample further comprises at least one first binding molecule specific for the at least one target, and at least one second binding molecule specific for the first binding molecule. It should be noted that the second binding molecule comprises at least one labeling moiety that comprises and/or produces at least one electroactive product. It should be appreciated that in some embodiments, the sample may be provided with these additional first and second binding molecules, however, the invention further encompasses methods that further comprise the step of adding the first and second binding molecules to the sample, and/or to the system or the second device. In yet some further embodiments, the sample is incubated with the second device for a sufficient time period. In yet some alternative embodiments, the method of the present disclosure may further comprise a washing step, where all unbound material is washed, and any first and second binding molecules present in the sample are bound to the target attached to the working electrode of the second device of the system of the invention. Still further, the next step (ii), applying voltage signal between the at least one working electrode and at least one reference electrode and determining electrical current through the at least one working electrode in response to varying voltage signal; and (iii) determining peak current value, the peak current value is inversely indicative of presence and/or quantity of the at least one target.

In some embodiments, where the sample is subjected to an electrochemical impedance spectroscopy (EIS) analysis. Accordingly, the method further comprising processing electrical impedance determined based on one or more voltage signal frequencies for determining charge transfer electrical resistance between the at least one working electrode and the at least one counter electrode, and determining presence of the at least one target in the sample whenever said charge transfer electrical resistance is greater than a predetermined threshold value.

Still further, determining the charge transfer electrical resistance comprises determining an electrical circuit model representing charge transfer between the electrodes, the electrical circuit may comprise capacitance model connected in parallel to inductance model and charge transfer electrical resistance model, thereby allowing to determine charge transfer electrical resistance in accordance with total impedance of the circuit.

In yet some further embodiments, where the method alternatively, or additionally, comprises subjecting wherein said sample is subjected to an electrochemical voltammetry or amperometry analysis, the method further comprises applying the peak current value determined for the sample on a predetermine standard curve for determining concentration of said at least one target in the sample.

According to such embodiments, where the second device and/or systems thereof is used by the methods of the invention, the at least one labeling moiety of the at least one second binding molecule, produces at least one electroactive product.

Still further, such labeling moiety of the second binding molecule added to the sample, comprises at least one enzyme that catalyzes the conversion of at least one substrate into at least one electroactive product.

In some embodiments of the disclosed methods, the enzyme is at least one of horseradish peroxidase (HRP), and alkaline phosphatase (ALP).

Still further, in some embodiments, the enzyme is HRP that catalyzes the oxidation of at least one substrate. More specifically, the at least one of the substrates of this enzyme is acetaminophen.

Thus, in some embodiments, the method, when using systems that comprise the second device, may further comprise the step of adding or providing the sample with an effective amount of acetaminophen.

In some alternative embodiments, where ALP is used as the labeling moiety, the method of the present disclosure further comprises the step of providing the sample with an effective amount of the substrate pAPP (para-aminophenol phosphate), that is hydrolyzed by ALP to yield pAP (para aminophenol) which is electroactive, specifically, it undergoes oxidation (1-electron oxidation) at low potentials (redox potential of +200 mV vs. Ag/AgCl reference electrode).

In yet some alternative embodiments of the disclosed methods, the second binding molecule provided with the sample where systems that comprise the second device are used, the at least one labeling moiety of such at least one second binding molecule comprises at least one electroactive product. In some specific embodiments, such labeling moiety is at least one Ferrocene molecule.

Still further, in some embodiments, the at least one first binding molecule is at least one primary antibody specific for the at least one target, and the at least one second binding molecule, is at least one secondary antibody specific for the primary antibody.

In some embodiments, the methods of the present disclosure are specifically applicable for identifying and/or quantifying and/or monitoring at least one target in a sample. In some embodiments, such target may be at least one small molecule compound.

In more specific embodiments, the methods of the present disclosure may be particularly useful for detecting small molecule compounds that may comprise at least one toxin.

In yet some further embodiments, such at least one toxin, may be any toxin produced by at least one pathogenic organism. In yet some further particular and non-limiting embodiments, such toxin may be any toxin produced by at least one bacterial cell. In some specific embodiments, the methods of the present disclosure may be particularly applicable for detecting, monitoring and/or quantitating at least one toxin, specifically small molecule toxin produced by cyanobacteria.

Thus, in some specific embodiments, the methods disclosed herein is applicable for detecting, monitoring and/or quantitating at least one toxin that may be at least one cyanotoxin.

Still further, in some embodiments, methods disclosed herein is applicable for detecting, monitoring and/or quantitating any cyanotoxin, for example, any cyanotoxin of any group, specifically, at least one of: at least one cyclic peptide, at least one alkaloid and at least one lipopolysaccharide, or any combinations thereof.

Still further, the cyanotoxin detectable by the methods of the present disclosure is at least one cyclic peptide. In yet some further embodiments, the cyclic peptide is at least one microcystin (MC), and at least one nodularin (NOD).

In some embodiments, the methods are applicable for detecting, monitoring and/or quantitating at least one toxin that is at least one microcystin. More specifically, in some embodiments, the microcystin is at least one of Microcystin-leucine-arginine (MC-LR), Microcystin-arginine-arginine (MC-RR), Microcystin-tyrosine-arginine (MC-YR), and Microcystin-leucine-alanine (MC-LA), and any combination, derivatives and variants thereof.

In some specific embodiments, the methods according to the present disclosure is applicable for detecting, monitoring and/or quantitating at least one microcystin, specifically, Microcystin-LR (MC-LR), or any derivatives and variants thereof. Thus, the invention provides methods using at least one biosensor chip system specifically applicable for detecting, monitoring and/or quantitating MC-LR.

In yet some further embodiments of the methods of the present disclosure, at least one of: (i) the at least one working electrode of the first chip device used by the methods is connected directly or indirectly to at least one antibody that specifically binds the at least one cyanotoxin; and (ii) the at least one working electrode of the second chip device used b the methods is connected directly or indirectly to the at least one cyanotoxin. Thus, the disclosed system may comprise working electrode/s that ae connected to antibody that recognizes the specific cyanotoxin, and operates via EIS, or alternatively, and/or additionally, working electrodes that are bound to cyanotoxin itself, and are operated via voltammetry or amperometry, and work in some embodiments in a competitive assay as discussed herein after.

In some embodiments, the methods of the present disclosure are applicable for any sample, specifically, any of the samples disclosed by the present disclosure. In some embodiments, the sample is an environmental sample or a biological sample.

In some embodiments, the environmental sample comprises at least one sample obtained from natural or artificial water reservoir, reclaimed water, and wastewater treatment and sewage treatment. The sample can be obtained from reclaimed water samples, i.e, wastewater that were treated in a water treatment facility and are re-used for various purposes, namely agriculture.

Still further, in some embodiments, the method of the present disclosure are performed using any of the systems defined by the present disclosure.

A further aspect of the present disclosure relates to a method of treating, preventing, ameliorating, reducing or delaying the onset of a disorder associated with exposure to at least one toxin in a subject in need thereof. In some embodiments, the method comprising:

First in step (a), classifying a subject as exposed to the toxin if the presence of the at least one toxin is determined in at least one biological sample of the subject, or in at least one environmental sample associated with the subject. In some embodiments, determination of the presence of the at least one toxin in the sample is performed by at least one of: (I) performing an electrochemical impedance spectroscopy (EIS) analysis of the sample, and/or (II), performing an electrochemical voltammetry or amperometry analysis of the sample. More specifically, in some embodiments, for classifying the subjects, the disclosed method may comprise the step of (I) performing an electrochemical impedance spectroscopy (EIS) analysis of the sample. In some embodiments, performing such analysis comprises: in a first step (i), contacting with the sample a first plurality of electrodes comprising at least one working electrode and at least one reference electrode, or any first chip device or system comprising the same. It should be noted that the at least one working electrode is connected directly or indirectly to at least one target binding site and/or moiety. The next step (ii), involves applying voltage signal between the at least one working electrode and the at least one reference electrode, and determining electrical current between the electrodes in response to the voltage signals for a selected number of one or more signal frequencies; and (iii), determining relations between electrical current response and voltage signal for the one or more signal frequencies; and determining electrical impedance between the at least one working electrode and the at least one counter electrode. It should be noted that the impedance variation being indicative of presence and/or quantity of the at least one target in the sample.

As discussed herein, in some embodiments, for classifying the subjects, the disclosed method may comprise either as an alternative step, or as an additional step, (II), performing an electrochemical voltammetry or amperometry analysis of the sample. In some embodiments, such additional and/or alternative analysis comprising: (i), contacting with the sample a second plurality of electrodes comprising at least one working electrode and at least one reference electrode, or any second chip device or system comprising the same. In some embodiments, at least one working electrode is connected directly or indirectly to the at least one target or any component thereof. Accordingly, the sample further comprises at least one first binding molecule specific for the at least one target, and at least one second binding molecule specific for the first binding molecule. It should be noted that the second binding molecule comprises at least one labeling moiety that comprises and/or produces at least one electroactive product. It should be appreciated that in some embodiments, the sample may be provided with these additional first and second binding molecules, however, the invention further encompasses methods that further comprise the step of adding the first and second binding molecules to the sample, and/or to the system or the second device. In yet some further embodiments, the sample is incubated with the second device for a sufficient time period. In yet some alternative embodiments, the method of the present disclosure may further comprise a washing step, where all unbound material is washed, and any first and second binding molecules present in the sample are bound to the target attached to the working electrode of the second device of the system of the invention. Still further, the next step (ii), applying voltage signal between the at least one working electrode and at least one reference electrode and determining electrical current through the at least one working electrode in response to varying voltage signal; and (iii) determining peak current value, the peak current value is inversely indicative of presence and/or quantity of the at least one target.

The next step (b), of the disclosed therapeutic methods administering to a subject classified as an infected subject in step (a), a therapeutically effective amount of at least one anti-toxin agent and/or additional therapeutic agent.

In some embodiments, determination of the presence of the at least one toxin in the sample is performed by the method as defined by the invention, and specified herein above.

In some embodiments, the toxin is cyanotoxin, preferably, MC-LR. Thus, the therapeutic methods may be applicable for treating disorders associated with exposure to the MC-LR. In some embodiments, such disorders may comprise at least one of liver damage, renal failure and neoplastic disorders.

It should be noted that in certain embodiments, disorders associated with cyanotoxins include poisoning of humans with cyanotoxins is possible through various pathways, mainly by the consumption of contaminated food (vegetables, fish, seafood, and livestock), as well by bathing and recreational activities with contaminated water. Still further, most of the microcystins have hydrophilic structure; thus, their cell uptake should be facilitated by transporting systems, such as the organic anion transporting polypeptides (OATPs). The fact that MC accumulation is primarily in the liver is explained by the amount of OATPs present in this organ, which is why MCs are considered as hepatotoxins. More specifically, the MC-LR has been determined as a substrate for OATP1A2, OATP1B1, and OATP1B3. MCs are established to require active transport for human cell uptake, and the high expression of these OATP1B1 and OATP1B3 transporters in the liver accounts for their selective liver toxicity. MC accumulation has been also shown as involved in liver, colon, and pancreatic tumors, as well as in hepatocellular carcinoma. Therefore, the therapeutic methods of the present disclosure that further comprise the diagnostic methods discussed herein, are applicable for these neoplastic disorders as well.

Still further aspect of the present disclosure relates to a method for identifying and/or quantifying at least one cyanotoxin in a sample, the method comprising:
contacting the sample with at least one working electrode, at least one reference electrode, and at least one counter electrode, or any biosensor chip or kit comprising said electrodes, wherein the at least one working electrode is connected directly or indirectly to at least one cyanotoxin binding site and/or moiety;
measuring electrical voltages between the at least one working electrode and said at least one reference electrode in response to electric currents of different frequencies applied between said at least one working electrode and the at least one reference electrode;
determining electrical impedances based on the measured electrical voltage and the electric currents applied at the different frequencies;
determining a charge transfer electrical resistance based on the determined impedances; and
determining presence of the at least one cyanotoxin in the sample whenever said charge transfer electrical resistance is greater than a predetermined threshold value.

In some embodiments, the determining a charge transfer electrical resistance comprises determining an electrical circuit model equivalent to a circuitry defined by the electrodes and the sample based on the determined electrical impedances.

Still further, the determining of the equivalent electrical circuit model comprises correlating Nyquist presentation of the electrical impedances determined at the different frequencies to Nyquist presentation of electrical impedances of the equivalent electrical circuit model.

In yet some further embodiments, the measurement chamber comprises a plurality of working electrodes, each connected directly or indirectly to at least one target binding site and/or moiety, and wherein the method comprising determining a respective plurality of electrical impedances associated with at least some of the plurality of working electrodes, and determining the charge transfer electrical resistance based of the determined respective plurality of electrical impedances.

Still further, the measurement chamber comprises a plurality of working electrodes, each connected directly or indirectly to at least one target binding site and/or moiety, and a respective plurality of reference electrodes, and wherein the method comprising determining a respective plurality of electrical impedances associated pairs of said working and reference electrodes, and determining the charge transfer electrical resistance.

It should be appreciated that any term disclosed by the present disclosure in connection with various aspects of the present disclosure is applicable for any of the aspects of the present invention, even if specified for other aspects.

Therefore, in some further aspects thereof, the present disclosure provides a biosensor chip device, specifically applicable for T3SS.

More specifically, a biosensor chip device usable for identifying and/or quantifying a target in a sample by electrochemical impedance spectroscopy (EIS) analysis. The chip device includes an arrangement of two or more electrodes configured to be in contact with a sample, typically within a measurement chamber. One of the two or more electrodes carries one or more binding sites, e.g., carrying antibodies such as the above described mAb-EspB-B7. The device of the present disclosure, or any system for measuring the electrical signal generated by the reaction product may further comprise a control module which may be a computer, electronic device/circuitry (e.g., a potentiostat) and may include one or more multiplexer modules for providing separation between plurality of measurement channels when used.

As indicated above, the biosensor chip disclosed herein is usable for identifying and/or quantifying a target in a sample. As used herein, in some embodiments, the target is any entity comprising a proteineous material recognized by the target binding site/entity of the disclosed biosensor chip. As used herein, proteineous material may comprise proteins, peptides and any amino acid sequence as disclosed herein after.

In yet some further embodiments, the target identified and/or quantified is a pathogen comprising at least one proteineous material recognized by the binding moiety of the working electrode of the disclosed device.

Still further, a target pathogen as used herein refers to any pathogenic agents include any pathogens, such as viruses, prokaryotic microorganisms, lower eukaryotic microorganisms, complex eukaryotic organisms, fungi, prions, parasites, yeasts, as well as toxins and venoms.

Of particular relevance are bacterial pathogens. A prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Pseudomonas sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum.

A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

In some further embodiments, viral pathogen/s may be detected and/or quantified by the biosensor chip of the present disclosure. The term “viruses” is used in its broadest sense to include viruses of the families adenoviruses, papovaviruses, herpesviruses: simplex, varicella-zoster, Epstein-Barr, CMV, pox viruses: smallpox, vaccinia, hepatitis B, rhinoviruses, coronaviruses, retroviruses, zika virus, Ebola virus, hepatitis A, poliovirus, rubella virus, hepatitis C, arboviruses, rabies virus, influenza viruses A and B, measles virus, mumps virus, HIV, HTLV I and II. The term “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis. The term “parasite” includes, but not limited to, infections caused by somatic tapeworms, blood flukes, tissue roundworms, ameba, and Plasmodium, Trypanosoma, Leishmania, and Toxoplasma species.

In some embodiments, the target detected and/or quantified by the chip device of the present disclosure is at least one pathogen expressing at least one component of the Type III Secretion System (T3SS).

Still further, in some embodiments, the chip device of the present disclosure comprises at least one target binding site and/or moiety that may be comprised within or comprises at least one antibody that recognizes and binds at least one proteineous component of any of the disclosed pathogens. In some embodiments, the chip device of the present disclosure comprises at least one target binding site and/or moiety that may be comprised within at least one antibody that recognizes and binds at least one component of the T3SS, or any combination or complex thereof. In certain specific embodiments, at least one antibody is used as a target binding site, such antibody or any functional fragments thereof is directly or indirectly immobilized in some embodiments to the at least one working electrodes. A wide range of Ab immobilization chemistries are applicable in the present disclosure, provided that they all must assure that the immobilized antibody strongly retained to the surface (the working electrode) in a functionally oriented fashion such that its antigen-binding sites are free to bind the antigen, that is the target discussed herein. Some include simply adsorption of the antibody onto the substrate after a prolonged incubation by passive adsorption. In yet some further embodiments, various functionalization and cross-linking strategies may be used, for example, those described by the present methods that include the direct covalent attachment of thiolated antibodies to a gold electrode surface. More specifically, the thiolation reaction is optimized to obtain an average of ˜6 —SH group per antibody by tuning the ratio of reagent to antibody. This fine-tuning enables control of the level of thiolation and ensures that antibody molecules are introduced with a sufficient number of thiols allowing their immobilization. Antibodies are thiolated in order to obtain a firm immobilization via gold-sulfur covalent bond, as discussed in the experimental procedures.

Still further, in some embodiments, the target binding site or moiety in the biosensor chip device of the present disclosure, is according to certain embodiments, at least one antibody that specifically recognizes and binds at least one component of the Type III Secretion System (T3SS) of at least one bacterium. Specifically, T3SS of Enteropathogenic Escherichia coli (EPEC).

The “Type III Secretion System or T3SS” is a complex structure composed of several subunits, which in turn are made up of approximately 20 bacterial proteins. The proteins that make up the T3SS apparatus are termed structural proteins. Additional proteins called “translocators” serve the function of translocating another set of proteins into the host cell cytoplasm. The translocated proteins are termed “effectors,” since they are the virulence factors that affect the changes in the host cells, allowing the invading pathogen to colonize, multiply, and in some cases chronically persist in the host. Briefly, the T3SS apparatus consists of two rings that provide a continuous path across the inner and outer membranes, including the peptidoglycan layer. The inner membrane ring is the larger of the two coaxial rings, and protein components that make up the inner ring have been identified for a number of bacteria. The outer membrane ring is composed of the secretin protein family, which is also known to be involved in type 2 secretion and in the assembly of type IV bacterial pili. A needle-like structure associates with the outer membrane ring and projects from the bacterial surface. It varies in length among the different pathogens and, in the case of pathogenic Escherichia coli, is extended by the addition of filaments that are thought to facilitate attachment to the host cells through the thick glycocalyx layer. Effectors are thought to be transported through the hollow tube-like needle into the host cell through the pores formed in the host cell membrane by the translocator proteins. Translocators are usually conserved among the different pathogens possessing a T3SS and show functional complementarity for secretion and translocation, whereas the effectors are most often distinct, having unique functions suited to a particular pathogen's virulence strategy. However, effector homologues also exist among different T3SS-possessing bacteria.

Still further, in some embodiments, the antibody comprised in the chip device of the present disclosure recognizes at least one component of the T3SS, for example, at least one of the Enteropathogenic Escherichia coli (EPEC) secreted protein A (EspA), EPEC secreted protein B (EspB), and EPEC secreted protein D (EspD), or any fragments or peptides thereof, and any combination or complex thereof.

In some embodiments, the chip device of the present disclosure comprises at least one antibody that recognizes and binds the EspB protein, or any fragments or peptides thereof, or any complex thereof with EspD protein.

In some embodiments, an antibody useful as a target binding site in the diagnostic biosensor chip devices, kits and methods of the invention may bind the Escherichia Coli secreted protein B (EspB) expressed by the bacterium, or any fragments or peptides thereof. Thus, in some embodiments, the diagnostic biosensor chip devices, kits and methods disclosed herein are used for detecting EspB expressing bacteria.

Among the virulence factors comprising the T3SS of these bacteria are the secreted proteins (Esps). The Esp responsible for the syringe-like structure of T3SS is secreted protein A (EspA), which is the needle-shaped protein of approximately 25 kDa, while secreted proteins B [Escherichia coli-secreted protein B (EspB)] and D [Escherichia coli-secreted protein D (EspD)] are responsible for the pore structure assembled in the eukaryotic membrane. Escherichia coli-secreted protein B is approximately 37 kDa in size and forms the pore assembled “needle tip” in the host cell membrane together with EspD. Also, EspB participates in phagocytosis evasion and binding to eukaryotic cell myosin, inhibition of actin interaction, and damage to the microvilli. There are three variants of EspB, i.e., α, β, and γ, where the α variant is subdivided into 1, 2, and 3. Allele frequency studies have shown α EspB to be the most prevalent, followed by β EspB.

In some embodiment, the EspB protein comprises the amino acid sequence as denoted by SEQ ID NO: 40 (Accession number: WP_001091991.1), or any homologs or derivatives thereof. In some specific embodiments, the EspB protein is encoded by a nucleic sequence as denoted by SEQ ID NO: 41 (Accession number: AAB69980.1), or any homologs or derivatives thereof.

In some further embodiments, the EspD protein comprises the amino acid sequence as denoted by SEQ ID NO: 42 (Accession number: WP_000935767.1), or any homologs or derivatives thereof. In some specific embodiments, the EspD protein is encoded by a nucleic sequence as denoted by SEQ ID NO: 43 (Accession number: CAI43861.1).

In some embodiments, the isolated antibody used in the diagnostic biosensor chip devices, kits and methods of the invention, specifically recognizes and binds an epitope comprising residues 185 to 250, specifically residues 190 to 215, more specifically, residues 193 to 210, of the EspB protein, specifically, the EspB protein that comprises the amino acid sequence as denoted by SEQ ID NO. 40. In yet some further embodiments, the epitope recognized by the antibody of the invention may comprise the amino acid sequence of TSAQKASQVAEEAADAAQ, or at least part thereof. In yet some further embodiments, the epitope recognized by the antibody of the invention may comprise the amino acid sequence as denoted by SEQ ID NO: 39.

In some specific embodiments, the chip device of the present disclosure comprises (optionally directly or indirectly immobilized therein) at least one antibody that recognizes and binds the EspB protein. In more specific and non-limiting embodiments, the antibody of the chip device of the present disclosure comprises a heavy chain complementarity determining region (CDRH) 1 comprising the amino acid sequence GFTFSHYA, as denoted by SEQ ID NO. 6, CDRH2 comprising the amino acid sequence INSNGDST, as denoted by SEQ ID NO. 10, CDRH3 comprising the amino acid sequence ARDRRAGYFDYW, as denoted by SEQ ID NO. 14, and a light chain complementarity determining region (CDRL) 1 comprising the amino acid sequence RDNIGKNY as denoted by SEQ ID NO. 22, a CDRL2 comprising the amino acid sequence RNN as denoted by SEQ ID NO. 26, and a CDRL3 comprising the amino acid sequence SAWDTSLNA as denoted by SEQ ID NO. 30, or any derivative, variant and biosimilar thereof. As used herein, the term “biosimilar” relates in some embodiments, to a biological product, for example, proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97 percent or greater to the amino acid sequence of its reference medicinal product, e.g., 97 percent, 98 percent, 99 percent or 100 percent. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product.

In some embodiments, the antibody may comprise a heavy chain complementarity determining region (CDRH) 1 comprising the amino acid sequence GFTFSHYA, as denoted by SEQ ID NO. 6, or any homologs or derivatives thereof, CDRH2 comprising the amino acid sequence INSNGDST, as denoted by SEQ ID NO. 10, or any homologs or derivatives thereof, CDRH3 comprising the amino acid sequence ARDRRAGYFDYW, as denoted by SEQ ID NO. 14, or any homologs or derivatives thereof, and a light chain complementarity determining region (CDRL) 1 comprising the amino acid sequence RDNIGKNY as denoted by SEQ ID NO. 22, or any homologs or derivatives thereof, a CDRL2 comprising the amino acid sequence RNN as denoted by SEQ ID NO. 26, or any homologs or derivatives thereof, and a CDRL3 comprising the amino acid sequence SAWDTSLNA as denoted by SEQ ID NO. 30, or any homologs or derivatives thereof, or any derivative, variant and biosimilar of the antibody of the invention.

In some embodiments, the antibody may comprise a heavy chain variable region and a light chain variable region, specifically, comprising CDR sequences as described above. In some specific embodiments, the heavy chain variable region is encoded by a nucleic acid sequence which is at least 70% identical to the nucleic acid sequence denoted by SEQ ID NO.1, or any homologs or derivatives thereof. In yet some further embodiments, the light chain variable region is encoded by a nucleic acid sequence which is at least 70% identical to SEQ ID NO.17, or any homologs or derivatives thereof.

In some other embodiments, the antibody may comprise a heavy chain variable region comprising the amino acid sequence denoted by SEQ ID NO.2 or any homologs, derivatives or variants thereof and a light chain variable region comprising the amino acid sequence denoted by SEQ ID NO.18 or any homologs, derivatives or variants thereof.

In more specific embodiments, the isolated monoclonal antibody or any antigen-binding fragment thereof may comprise a Heavy chain Framework Region 1 (FR1) comprising the amino acid sequence denoted by SEQ ID NO: 4, or any homologs or derivatives thereof, a heavy chain FR2 comprising the amino acid sequence denoted by SEQ ID NO: 8, or any homologs or derivatives thereof and a heavy chain FR3 comprising the amino acid sequence denoted by SEQ ID NO: 12, or any homologs or derivatives thereof, and a Light chain Framework Region 1 (FR1) comprising the amino acid sequence denoted by SEQ ID NO: 20, or any homologs or derivatives thereof, a Light chain FR2 comprising the amino acid sequence denoted by SEQ ID NO: 24, or any homologs or derivatives thereof, and a Light chain FR3 comprising the amino acid sequence denoted by SEQ ID NO: 28, or any homologs or derivatives thereof.

The term “antibody” as used herein, means any antigen-binding molecule or molecular complex that specifically binds to or interacts with a particular antigen of any fragments thereof. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region (CH). The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. Typically, an antibody is composed of two immunoglobulin (Ig) heavy chains and two Ig light chains. In humans, antibodies are encoded by three independent gene loci, namely kappa (κ) chain (Igκ) and lambda (λ) chain (Igλ) genes for the Light chains and IgH genes for the Heavy chains, which are located on chromosome 2, chromosome 22, and chromosome 14, respectively.

The antibody of the invention may be a monoclonal antibody, and in some embodiments a humanized or human antibody or any antigen-binding fragment thereof. In some embodiments, the antibody of the invention is a monoclonal antibody. A monoclonal antibody, as used herein refers to an antibody produced by a single clone of cells or cell line producing identical antibody molecules. Monoclonal antibodies display monovalent affinity in binding the same epitope. It should be further understood that the present invention further encompasses any functional fragments of then antibody of the invention, such fragments are referred to herein as antigen binding fragments. The term “an antigen-binding fragment” refers to any portion of an antibody that retains binding to the antigen.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

Examples of antibody functional fragments include, but are not limited to a single-domain antibody (sdAb) which refers to an antibody fragment consisting of a single monomeric variable antibody domain. The first single-domain antibodies were engineered from heavy-chain antibodies found in camelids; these are called VHH fragments. Cartilaginous fishes also have heavy-chain antibodies (IgNAR, ‘immunoglobulin new antigen receptor’), from which single-domain antibodies called variable new antigen receptor antibody (V-NAR) fragments can be obtained.

An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes. Thus, it should be further appreciated that in some embodiments, the invention further encompasses a polypeptide comprising a variable region of a light chain comprising at least one of the CDR comprising the amino acid sequences as denoted by SEQ ID NO. 22, 26 and 30, or any homologs or derivatives thereof. In yet some further embodiments, the polypeptide of the invention may comprise the sequence of a variable region, as denoted by SEQ ID NO. 18, or any homologs thereof. In yet some further embodiments, the invention further provides a polypeptide comprising a variable region of an antibody heavy chain. In some specific embodiments, such polypeptide may comprise the amino acid sequence of at least one of the following CDRs, specifically, CDRs comprising the amino acid sequences as denoted by any one of SEQ ID NO. 6, 10 and 14, or any homologs or derivatives thereof. In yet some further embodiments, the polypeptide of the invention may comprise the variable region of the heavy chain as denoted by SEQ ID NO. 2, or any homologs or derivatives thereof.

As appreciated by one of skill in the art, various antibody fragments can be obtained by a variety of methods, for example, digestion of an intact antibody with an enzyme, such as pepsin, or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries. The term antibody also includes multivalent antibodies, specifically, bivalent molecules, diabodies, triabodies, tetrabodies and the like.

References to “V_{H}” or a “VH” refer to the variable region of an immunoglobulin heavy chain, including an Fv, scFv, a disulfilde-stabilized Fv (dsFv) or Fab. References to “V_{L}” or a “VL” refer to the variable region of an immunoglobulin light chain, including of an Fv, scFv, dsFv or Fab.

More specifically, the phrase “single chain Fv” or “scFv” refers to an antibody in which the variable domains of the heavy chain and of the light chain of a traditional two chain antibody have been joined to form one chain. Typically, a linker peptide is inserted between the two chains to allow for the stabilization of the variable domains without interfering with the proper folding and creation of an active binding site. A single chain antibody applicable for the invention, e.g., may bind as a monomer. Other exemplary single chain antibodies may form diabodies, triabodies, and tetrabodies.

It should be appreciated that in some embodiments, any antibody provided by the present disclosure and used by the diagnostic biosensor chip device, methods, and kits of the present disclosure is not a naturally occurring antibody. Specifically, any of the antibodies used herein cannot be considered as a product of nature. In yet some further embodiments, it should be noted that the epitope recognized by the antibodies of the invention may comprise, at least part of residues 185 to 250, specifically, residues 190 to 215, more specifically, 193 to 210 of the EspB protein, specifically, the EspB as denoted by SEQ ID NO. 40. Still further, in some embodiments, the antibody of the invention comprises at least part of the amino acid sequence TSAQKASQVAEEAADAAQ, as denoted by SEQ ID NO. 39. According to Donnenberg et al. (Donnenberg et a. (2011) Journal of Bacteriology; p 2972-2980), the EspB protein adopts a transmembrane topology with its C-terminus facing the host cytoplasm. Therefore, the epitope should be found inside the host cell following bacterial infection. It should be appreciated that the invention further encompasses in some embodiments thereof any antibody that recognizes and binds an epitope comprising the amino acid sequence as denoted by SEQ ID NO. 39, or any homologs or derivatives thereof.

The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three-dimensional structural characteristics as well as specific charge characteristics.

In yet some further embodiments, the antibody of the invention cannot be considered as naturally occurring antibody. As such, the antibody of the invention is not a product of nature.

Still further, it should be understood, that in some embodiments thereof, the invention further encompasses the use of any antibody that competes with any of the antibodies disclosed herein, specifically, any antibody that competes with an antibody comprising at least one of the CDRs as denoted by SEQ ID NO. 6, 10, 14, 22, 26 and 30, or any homologs or derivatives thereof. In yet some further embodiments, the invention further encompasses any antibody that competes with an antibody comprising the variable heavy chain as denoted by SEQ ID NO. 2, or any homologs or derivatives thereof, and/or the variable light chain that comprises the amino acid sequence as denoted by SEQ ID NO. 18, or any homologs or derivatives thereof. In yet some further embodiments, the term “competes” as used herein refers to any competition that results in reduction, attenuation, decrease or inhibition of binding of at least one of, the binding of the antibody of the invention to its epitope.

The invention relates to the use of antibodies that are polypeptides comprising amino acid sequences. “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

It should be appreciated that the invention encompasses the use of any variant or derivative of the antibody of the invention and any antibodies that are substantially identical or homologue to the antibodies encoded by the nucleic acid sequence of the invention. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof. Proteins orthologs or homologues having a sequence homology or identity to the proteins of interest in accordance with the invention, specifically antibodies described herein, may share at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the proteins of interest in accordance with the invention, for example, any of the antibodies that comprise the amino acid sequence as denoted by any one of SEQ ID NO. 2 and 18, or any one of the CDRs of SEQ ID NO. 6, 10, 14, 22, 26 and 30. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO. 2 and 18 specifically, the entire sequence as denoted by SEQ ID NO. 2 and 18, or any one of the CDRs of SEQ ID NO. 6, 10, 14, 22, 26 and 30.

In some embodiments, derivatives refer to antibodies, which differ from the antibodies specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

Variants of the antibodies of the invention may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the antibodies of the invention.

In some embodiments, the invention relates to a biosimilar derived from the mAb-B7 antibody described above.

In some embodiments, the chip device of the present disclosure is usable for detecting the presence of a pathogen expressing at least one T3SS component in a sample. In some embodiments, such pathogen is a bacterial pathogen. In yet some further embodiments, the bacterium is at least one Multiple Drug Resistant (MDR) bacterium.

In more specific embodiments, the MDR bacterium is at least one of Enteropathogenic Escherichia coli (EPEC) and Enterohemorrhagic Escherichia coli (EHEC).

In some embodiments, a sample that may be used for the chip device of the present disclosure may be a biological sample or an environmental sample, as will be described herein after.

A further aspect of the inventio relates to a kit comprising:

First (a), at least one biosensor chip device usable for identifying and/or quantifying a target in a sample by electrochemical impedance spectroscopy (EIS) analysis, the chip device comprising:

An arrangement of two or more electrodes configured to be in contact with a sample within a measurement chamber and to be connectable to an electronic device for enabling EIS measurement between the electrodes; wherein at least one of said electrodes is connected directly or indirectly to at least one target binding site and/or moiety.

In some embodiments the arrangement of two or more electrodes may be carried by a substrate, such as printed circuit.

In some embodiments, the measurement chamber may be formed by a packaging assembly configured to sealably enclose said electrodes portion of the substrate and define a measurement chamber encompassing said electrodes.

In some embodiments, the kit of the present disclosure optionally further comprises at least one of: (b) at least one control sample and/or control standard value, and (c) instructions for use.

In some embodiments, the kit disclosed herein may comprise at least one biosensor chip device as defined by the present disclosure.

A further aspect of the present disclosure relates to a method for identifying and/or quantifying at least one target in a sample, the method comprising:
providing an electrode arrangement comprising at least one counter electrode and at least one working electrode within connection with the sample, wherein said at least one working electrode carries at least one target binding site and/or moiety;
applying voltage signal between said at said least one working electrode and said at least one counter electrode, and determining current response between the electrodes for a selected number of one or more signal frequencies;
utilizing a relation between current response and voltage signal and determining electrical impedance between the working electrode and counter electrode; impedance variation being indicative of presence and concentration of said at least one target in said sample.

It should be noted that in some embodiments, voltage between the electrodes may be determined in response to current signal driven therebetween in one or more selected frequencies.

In some embodiments, the method further comprises using one or more computer processor for processing electrical impedance determined based on one or more voltage signal frequencies for determining charge transfer electrical resistance between the working and counter electrodes, and determining presence of said at least one target in said sample based on said charge transfer electrical resistance. Generally, presence of the at least one target may be determined in accordance with a look-up table and/or predetermined threshold limits selected in accordance with data on said sample and said at least one target.

Generally, the voltage signal may be in the form of alternating voltage signals, e.g., sinusoidal wave, having one or more selected frequencies. Electrical impedance between the electrodes may be determined in accordance with magnitude of current response and phase shift between the current response and the voltage signal.

Generally, the charge transfer electrical resistance may be determined in accordance with a electrical circuit model representing charge transfer between the electrodes, such electrical circuit may comprise capacitance model connected in parallel to inductance model and charge transfer electrical resistance model, thereby allowing to determine charge transfer electrical resistance in accordance with total impedance of the circuit.

A yet further aspect of the present disclosure relates to a method for identifying and/or quantifying at least one target in a sample. More specifically, the method comprising the following steps:

The first step involves contacting at least one sample with at least one working electrode, at least one reference electrode, and at least one counter electrode or any biosensor chip or kit comprising the electrodes. It should be noted that the at least one working electrode is connected directly or indirectly to at least one target binding site and/or moiety.

The next step involves measuring electrical voltages between the at least one working electrode and the at least one reference electrode in response to electric currents of different frequencies applied by the at least one counter electrode.

The next step involves determining electrical impedances based on the measured electrical voltage and the electric currents applied at the different frequencies.

In the next step, determining a charge transfer electrical resistance based on the determined impedances is performed.

The following step involves determining presence of the target in the sample whenever the charge transfer electrical resistance determined in the previous step is greater than a predetermined threshold value.

In some embodiments, determining a charge transfer electrical resistance by the method of the invention as indicated above, may comprise determining an electrical circuit model equivalent to a circuitry defined by the electrodes and the sample based on the determined electrical impedances. Such electrical circuit model may be associated with capacitance, inductance and resistance parameters, where at least a portion of total resistance model is associated with said charge transfer electrical resistance.

Still further, in some embodiments of the methods of the present disclosure, the determining of the equivalent electrical circuit model comprises correlating Nyquist presentation of the electrical impedances determined at the different frequencies to Nyquist presentation of electrical impedances of the equivalent electrical circuit model.

In yet some further embodiments, the measurement chamber used by the methods of the present disclosure comprises a plurality of working electrodes, each connected directly or indirectly to at least one target binding site and/or moiety. More specifically, the method comprising determining a respective plurality of electrical impedances associated with at least some of the plurality of working electrodes, and determining the charge transfer electrical resistance based of the determined respective plurality of electrical impedances.

In some embodiments, the measurement chamber comprises a plurality of working electrodes, each connected directly or indirectly to at least one target binding site and/or moiety, and a respective plurality of reference electrodes. Still further, according to these embodiments, the method comprising determining a respective plurality of electrical impedances associated pairs of said working and reference electrodes, and determining the charge transfer electrical resistance based of the determined respective plurality of electrical impedances.

In some embodiments, the target detected and/or quantified by the methods of the present disclosure is at least one pathogen expressing at least one component of the Type III Secretion System (T3SS).

In yet some further embodiments, at least one target binding site and/or moiety used by the methods of the present disclosure is comprised within at least one antibody that recognizes and binds at least one component of the T3SS, or any combination or complex thereof. In more specific embodiments, the antibody or any functional fragments thereof is immobilized to at least one of the working electrode/s used by the methods of the present disclosure.

In more specific embodiments, the antibody used by the disclosed methods recognizes at least one component of T3SS, for example, at least one of the Enteropathogenic Escherichia coli (EPEC) secreted protein A (EspA), EPEC secreted protein B (EspB), and EPEC secreted protein D (EspD), and any combination or complex thereof.

In yet some further embodiments, the antibody used by the disclosed method may be at least one antibody recognizes and binds the EspB protein, or any complex thereof with EspD protein.

In more specific embodiments, the method of the present disclosure may use at least one antibody that recognizes and binds the EspB protein. In more specific embodiments, such antibody comprises a heavy chain complementarity determining region (CDRH) 1 comprising the amino acid sequence GFTFSHYA, as denoted by SEQ ID NO. 6, CDRH2 comprising the amino acid sequence INSNGDST, as denoted by SEQ ID NO. 10, CDRH3 comprising the amino acid sequence ARDRRAGYFDYW, as denoted by SEQ ID NO. 14, and a light chain complementarity determining region (CDRL) 1 comprising the amino acid sequence RDNIGKNY as denoted by SEQ ID NO. 22, a CDRL2 comprising the amino acid sequence RNN as denoted by SEQ ID NO. 26, and a CDRL3 comprising the amino acid sequence SAWDTSLNA as denoted by SEQ ID NO. 30, or any derivative, variant and biosimilar thereof.

In some embodiments, the method disclosed herein is intended for detecting at least one pathogen in a sample. In some embodiments, such pathogen is a bacterial pathogen. In yet some further embodiments, the bacterium is at least one Multiple Drug Resistant (MDR) bacterium.

The present disclosure therefore provides diagnostic biosensor chip devices, kits and methods for detecting T3SS expressing bacteria in a sample. It should be noted that the term “bacterium” or “bacteria” as used herein refers to any of the prokaryotic microorganisms that exist as a single cell or in a cluster or aggregate of single cells. In more specific embodiments, the term “bacteria” specifically refers to Gram negative bacteria, or a Gram-positive bacteria, specifically, a Gram negative bacteria. In some embodiments, the at least one bacterium referred herein may be a gram-negative bacteria.

While the Gram-positive bacteria are recognized as retaining the crystal violet stain used in the Gram staining method of bacterial differentiation, and appear to be purple-colored under a microscope, the Gram-negative bacteria do not retain the crystal violet, making positive identification possible. In other words, the term bacteria apply herein to bacteria with a thin peptidoglycan layer of their cell wall that is sandwiched between an inner cytoplasmic cell membrane and a bacterial outer membrane (Gram-negative).

In yet some other embodiments, the bacterium relevant to the antibody of the invention may be at least one Multiple Drug Resistant (MDR) bacterium.

As used herein, the term “resistance” is not meant to imply that the bacterial cell population is 100% resistant to a specific antibiotic compound, but includes bacteria that are tolerant of the antibiotics or any derivative thereof. More specifically, the term “bacterial resistance gene/s” refers to gene/s conferring about 100%, 90%, 80%, 70%, 60%, 50%, 40%, 30%, 20%, or 10% protection from an antibiotic compound, thereby reversing susceptibility and sensitivity thereof to said antibiotic compound.

Bacteria of particular interest may be any bacteria involved in nosocomial infections or any mixture of such bacteria. The term “Nosocomial Infections” refers to Hospital-acquired infections, namely, an infection whose development is favored by a hospital environment, such as surfaces and/or medical personnel, and is acquired by a patient during hospitalization. Nosocomial infections are infections that are potentially caused by organisms resistant to antibiotics. Nosocomial infections have an impact on morbidity and mortality, and pose a significant economic burden. In view of the rising levels of antibiotic resistance and the increasing severity of illness of hospital in-patients, this problem needs an urgent solution. Common nosocomial organisms include Clostridium difficile, methicillin-resistant Staphylococcus aureus, coagulase-negative Staphylococci, vancomycin-resistant Enteroccocci, resistant Enterobacteriaceae, Pseudomonas aeruginosa, Acinetobacter and Stenotrophomonas maltophilia.

The nosocomial-infection pathogens may be Gram-negative rod-shaped organisms (Klebsiella pneumonia, Klebsiella oxytoca, Escherichia coli, Proteus aeruginosa, Serratia spp.), Gram-negative bacilli (Enterobacter aerogenes, Enterobacter cloacae), aerobic Gram-negative coccobacilli (Acinetobacter baumanii, Stenotrophomonas maltophilia) and Gram-negative aerobic bacillus (Stenotrophomonas maltophilia, previously known as Pseudomonas maltophilia). Among many others Pseudomonas aeruginosa is an extremely important nosocomial Gram-negative aerobic rod pathogen.

“ESKAPE” pathogens may also be of particular interest. As indicated herein, these pathogens include but are not limited to Enterococcus faecium, Staphylococcus aureus, Klebsiella pneumoniae, Acinetobacter baumanii, Pseudomonas aeruginosa, and Enterobacter.

In further embodiments, the bacteria as referred to herein by the invention may include Yersinia enterocolitica, Yersinia pseudotuberculosis, Salmonella typhi, Pseudomonas aeruginosa, Vibrio cholerae, Shigella sonnei, Bordetella Pertussis, Plasmodium falciparum, Chlamydia trachomatis, Bacillus anthracis, Helicobacter pylori and Listeria monocytogens.

In some other specific embodiments, the bacterium referred herein may be a gram negative. In other specific embodiments, the target cells of interest may be any E. coli strain, specifically, any one of O157:H7, enteroaggregative (EAEC), enterohemorrhagic (EHEC), enteroinvasive (EIEC), enteropathogenic (EPEC), enterotoxigenic (ETEC) and diffuse adherent (DAEC) E. coli.

In some further embodiments, the MDR bacterium detected by the diagnostic biosensor chip devices, kits and methods of the present disclosure may be least one of Enteropathogenic Escherichia coli (EPEC) and Enterohemorrhagic Escherichia coli (EHEC). Enteropathogenic Escherichia coli and EHEC are the main bacterial agents associated with diarrhea among children under 5 years old, and both pathogens are able to induce the A/E lesion.

In some embodiments, the MDR bacterium may be Enteropathogenic Escherichia coli (EPEC). In some other embodiments, the MDR bacterium may be C. rodentium.

In more specific embodiments, the MDR bacterium is at least one of Enteropathogenic Escherichia coli (EPEC) and Enterohemorrhagic Escherichia coli (EHEC).

In some further embodiments, the antibody of the biosensor chip device, kits and methods of the present disclosure recognizes and binds at least one component of the T3SS of at least one MDR bacterium, and therefore provides the diagnosis of an MDR bacteria in a sample, or in a subject.

In some further embodiments, the MDR bacterium may be at least one of EPEC and EHEC.

Specifically concerning the EPEC and EHEC bacteria, the hallmark of EPEC and EHEC-induced intestinal pathology is the attaching and effacing (A/E) lesion, whose formation depends on a T3SS encoded within the loci of enterocyte effacement (LEE) and the interplay of many T3SS effectors. Following intimate attachment of the bacteria to the intestinal epithelium, the brush border microvilli are disrupted (effacement), and the bacteria promote formation of actin pedestals that elevate the pathogen above the intestinal epithelium. To attach to the enterocytes, EPEC and EHEC utilize their T3SSs to inject the Translocated Intimin Receptor (Tir) into the host cell, where it inserts into the host cell membrane and binds to the bacterial outer membrane protein intimin. Binding of intimin to Tir induces Tir clustering, initiating a cascade of signaling events that leads to actin polymerization and pedestal formation. This ultimately results in the formation of the A/E lesion. EPEC Tir is tyrosine phosphorylated to recruit the Arp2/3 complex and drive actin polymerization, whereas EHEC Tir is not phosphorylated but, rather, relies on an additional T3SS effector, TccP/EspFU, for Arp2/3 recruitment. Successful pedestal formation requires downregulation of filopodia, which form in response to EPEC/EHEC infection, as well as disruption of the host microtubule network. The T3SS effectors Map (mitochondrion-associated protein), Tir, EspH (153), EspG, and EspG2 mediate these processes. This multifaceted approach allows A/E pathogens to coordinate the formation of A/E lesions and actin pedestals, providing them with a unique niche in the intestine of the infected host.

In some more specific embodiments, the T3SS recognized by the antibody used by the methods of the invention may be an MDR bacterium, in some specific embodiments, such bacteria may be Enteropathogenic Escherichia coli (EPEC).

In yet another embodiment, the bacteria may induce attaching and effacing (A/E) lesion in the subject.

In some further embodiments, the bacteria referred herein may be C. rodentium.

In some further embodiments, the antibody used in the biosensor chip device, kits and methods of the invention may recognize the EspB expressed by the bacteria, as specified above.

In some embodiments, the methods of the invention may use, and thus may be applicable for identifying and/or quantifying of a target in a biological sample or an environmental sample.

The terms “sample”, “test sample” and “specimen”, “biological sample” are used interchangeably in the present specification and claims and are used in its broadest sense. They are meant to include both biological and environmental samples and may include an exemplar of synthetic origin. This term refers to any media that may contain the T3SS expressing bacteria and may include body fluids (urine, blood, milk, cerebrospinal fluid, rinse fluid obtained from wash of body cavities, phlegm, pus), samples taken from various body regions (throat, vagina, ear, eye, skin, sores), food products (both solids and fluids) and swabs taken from medicinal instruments, apparatus, materials), as well as substances in which controlled chemical reactions are being carried out. More specifically, according to certain embodiments, the method of the invention uses any appropriate biological sample. The term “biological sample” in the present specification and claims is meant to include samples obtained from any subject or environmental sources, for example, a mammal subject. It should be recognized that in certain embodiments a biological sample may be for example, blood cells, blood, serum, plasma, bone marrow, lymph fluid, urine, sputum, saliva, feces, semen, spinal fluid or CSF, the external secretions of the skin, respiratory, intestinal, and genitourinary tracts, tears, milk, any human organ or tissue, any sample obtained by lavage, optionally of the breast ducal system, plural effusion, sample of in vitro or ex vivo cell culture and cell culture constituents.

In certain embodiment, the biological sample suitable for the method of the invention may be any one of serum, whole blood sample, urine, saliva, or any fraction or preparation thereof.

In some embodiments, the sample applicable in the biosensor chip device, kits and methods of the invention may be either as naturally obtained from the tested subject or manipulated and prepared. In some embodiments, the body fluid samples may be concentrated samples. In yet some further embodiments, the serum samples may be diluted and as such, different sera concentrations may be used. In some further embodiments the serum concentration may range between about 0.01% and 100%, More specifically, 0.01%, 0.02%, 0.03%, 0.04%, 0.05%, 0.06%, 0.07%, 0.08%, 0.09%, 0.1%, 0.2%, 0.2%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20%, 25%, 30%, 35%, 40%, 45%, 50%, 55%, 60%, 65%, 70%, 75%, 80%, 85, 90%, 95%, 100% or more. In more specific embodiment, the sample concentration may range between about 1% to about 20%, in yet some further particular embodiments, the sample concentration of the sample may be 5%.

It should be further noted that in some embodiments, the diagnostic biosensor chip device, kits and methods of the invention may be also applicable for environmental samples. Environmental samples include environmental material such as surface matter, earth, soil, water, air and industrial samples, as well as samples obtained from food and dairy processing instruments, apparatus, equipment, utensils, disposable and non-disposable items. These examples are not to be construed as limiting the sample types applicable to the present invention. The sample may be any media, specifically, a liquid media that may contain the T3SS expressing bacteria. Typically, substances and samples or specimens that are a priori not liquid may be contacted with a liquid media which is contacted with the biosensor chip device of the invention.

More specifically, by the term “food”, it is referred to any substance consumed, usually of plant or animal origin. Some non-limiting examples of animals used for feeding are cows, pigs, poultry, etc. The term food also comprises products derived from animals, such as, but not limited to, milk and food products derived from milk, eggs, meat, etc.

In some specific embodiments, the present invention encompasses samples of a substance, which is used as a drink. A drink or beverage is a liquid which is specifically prepared for human consumption. Non limiting examples of drinks include, but are not limited to water, milk, alcoholic and non-alcoholic beverages, soft drinks, fruit extracts, etc.

In yet some further embodiments, the method of the invention, that detects, identify and/or quantify at least one pathogen in a sample, is used for the diagnosis of an infectious condition caused by or associated with at least one T3SS expressing pathogen, in a subject. According to these embodiments, the sample used by he disclosed method is at least one sample of the subject.

The present disclosure therefore provides a powerful diagnostic tool for rapid diagnosis of patients suffering from infectious condition caused by, or associated with, at least one bacterium expressing at least one T3SS.

The clinical spectrum of disease caused by T3SS-containing pathogens is remarkably broad. Infection with enteropathogenic and enterohemorrhagic E. coli (EPEC and EHEC, respectively), Shigella, Salmonella, and Yersinia species results in intestinal disease. Yersinia pestis is the causative agent of plague. Salmonella serovar Typhi causes enteric fever. Bordetella causes whooping cough, while the opportunistic pathogen Pseudomonas aeruginosa can cause a variety of problems, including pneumonia, urinary tract infection, wound infection, septicemia, and endocarditis. Chlamydia trachomatis is a common sexually transmitted organism, and Chlamydia pneumoniae causes pneumonia and has been implicated in atherosclerotic disease of blood vessels. Burkholderia pseudomallei causes community-acquired bacteremia and pneumonia. Whether by a direct toxic mechanism or through induction of self-damaging host responses, the virulence of all of these bacteria utilizes T3SSs. Clearly, T3SSs are not restricted to a specific pathogen, tissue, host environment, clinical disease spectrum, or patient population.

In some further embodiments, the diagnostic methods of the invention may be applicable for diagnosing any infection associated with at least one of transient enteritis or colitis, cholecystitis, bacteremia, cholangitis, urinary tract infection (UTI), traveler's diarrhea, neonatal meningitis and pneumonia, or any conditions, symptoms or effects associated therewith.

Thus, in some specific embodiments, the biosensor chip device, kits and methods of the invention may be applicable for transient enteritis. The term “transient enteritis or colitis” relates to an inflammation of the small intestine. It is most commonly caused by food or drink contaminated with pathogenic microbes. Duodenitis, jejunitis and ileitis are subtypes of enteritis which are only localized to a specific part of the small intestine. Inflammation of both the stomach and small intestine is referred to as gastroenteritis. Signs and symptoms of enteritis are highly variable and vary based on the specific cause and other factors such as individual variance and stage of disease. Symptoms may include abdominal pain, cramping, diarrhoea, dehydration, fever, nausea, vomiting and weight loss.

In yet some further embodiments, the biosensor chip device, kits and methods of the invention may be applicable for Cholecystitis. As used herein, Cholecystitis is inflammation of the gallbladder. Symptoms include right upper abdominal pain, nausea, vomiting, and occasionally fever. Often gallbladder attacks (biliary colic) precede acute cholecystitis. Complications of acute cholecystitis include gallstone pancreatitis, common bile duct stones, or inflammation of the common bile duct.

In some further embodiments, the biosensor chip device, kits and methods of the invention may be applicable for Bacteremia. Bacteremia (also bacteraemia) refers to the presence of bacteria in the blood. Bacteria can enter the bloodstream as a severe complication of infections (like pneumonia or meningitis), during surgery (especially when involving mucous membranes such as the gastrointestinal tract), or due to catheters and other foreign bodies entering the arteries or veins (including during intravenous drug abuse). Transient bacteremia can result after dental procedures or brushing of teeth.

Bacteremia can have several important health consequences. The immune response to the bacteria can cause sepsis and septic shock, which has a high mortality rate. Bacteria can also spread via the blood to other parts of the body (which is called hematogenous spread), causing infections away from the original site of infection, such as endocarditis or osteomyelitis.

Still further, in some embodiments, the biosensor chip device, kits and methods of the invention may be applicable for cholangitis. Ascending cholangitis, also known as acute cholangitis or cholangitis, is inflammation of the bile duct, usually caused by bacteria ascending from its junction with the duodenum (first part of the small intestine). It tends to occur if the bile duct is already partially obstructed by gallstones. Characteristic symptoms include yellow discoloration of the skin or whites of the eyes, fever, abdominal pain, and in severe cases, low blood pressure and confusion.

In yet some further embodiments, the biosensor chip device, kits and methods of the invention may be applicable for urinary tract infection. A urinary tract infection (UTI) is an infection that affects part of the urinary tract. When it affects the lower urinary tract it is known as a bladder infection (cystitis) and when it affects the upper urinary tract it is known as kidney infection (pyelonephritis). Symptoms from a lower urinary tract include pain with urination, frequent urination, and feeling the need to urinate despite having an empty bladder. Symptoms of a kidney infection include fever and flank pain usually in addition to the symptoms of a lower UTI. In some cases, the urine may appear bloody.

In certain embodiments, the biosensor chip device, kits and methods of the invention may be applicable for Traveler's diarrhea. Traveler's diarrhea (TD) is a stomach and intestinal infection. TD is defined as the passage of unformed stool (one or more by some definitions, three or more by others) while traveling. It may be accompanied by abdominal cramps, nausea, fever, and bloating. Occasionally bloody diarrhea may occur. Most travelers recover within four days with little or no treatment. About 10% of people may have symptoms for a week. Bacteria are responsible for more than half of cases. The bacteria enterotoxigenic Escherichia coli (ETEC) are typically the most common except in Southeast Asia, where Campylobacter is more prominent.

In yet some more embodiments, the biosensor chip device, kits and methods of the invention may be applicable for Neonatal meningitis. Neonatal meningitis is a serious medical condition in infants. Meningitis is an inflammation of the meninges (the protective membranes of the central nervous system (CNS)) and is more common in the neonatal period (infants less than 44 days old) than any other time in life and is an important cause of morbidity and mortality globally. Symptoms seen with neonatal meningitis are often unspecific that may point to several conditions, such as sepsis (whole body inflammation). These can include fever, irritability, and dyspnea. The only method to determine if meningitis is the cause of these symptoms is lumbar puncture (LP; an examination of the cerebrospinal fluid). The most common causes of neonatal meningitis is bacterial infection of the blood, known as bacteremia (specifically Group B Streptococci (GBS; Streptococcus agalactiae), Escherichia coli, and Listeria monocytogenes). Delayed treatment of neonatal meningitis may cause include cerebral palsy, blindness, deafness, and learning deficiencies.

In some embodiments, the biosensor chip device, kits and methods of the invention may be applicable for Pneumonia. Pneumonia is an inflammatory condition of the lung affecting primarily the small air sacs known as alveoli. Typically, symptoms include some combination of productive or dry cough, chest pain, fever, and trouble breathing. Bacteria are the most-common cause of community-acquired pneumonia (CAP), with Streptococcus pneumoniae isolated in nearly 50% of cases. Other commonly-isolated bacteria include Haemophilus influenzae in 20%, Chlamydophila pneumoniae in 13%, and Mycoplasma pneumoniae in 3% of cases; Staphylococcus aureus; Moraxella catarrhalis; Legionella pneumophila; and Gram-negative bacilli. A number of drug-resistant versions of the above infections are becoming more common, including drug-resistant Streptococcus pneumoniae (DRSP) and methicillin-resistant Staphylococcus aureus (MRSA).

It should be understood that the diagnostic methods disclosed by the invention may be further used for monitoring subjects treated with any therapeutic compound.

More specifically, the diagnostic methods of the invention may be further used form monitoring the extent of infection (or bacterial load) in the treated subject. For such monitoring purpose, the steps of the methods of the invention may be repeated at least one further time for at least one further sample obtained from the subject. In some embodiments, the sample is obtained in another time point and is therefore considered herein as a temporally separated sample. As indicated above, in accordance with some embodiments of the invention, in order to assess the patient condition, or monitor the disease progression, as well as responsiveness to a certain treatment, at least two “temporally-separated” test samples must be collected from the examined patient and compared thereafter in order to obtain the rate of change in the amount of bacteria between said samples, as reflected by the amount of T3SS component (e.g., the EspB protein) measured and determined by the biosensor chip device, kits and methods of the invention. In practice, to detect a change in at least one of these parameters between said samples, at least two “temporally-separated” test samples and preferably more must be collected from the patient.

This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least three days, at least one week, at least two weeks, at least three weeks, at least one month, at least two months, at least three months, at least four months, at least five months, at least one year, or even more.

When calculating the rate of change in the amount of the detected T3SS component (e.g., EspB), one may use any two samples collected at different time points from the patient. In some embodiments, at least one of the samples may be obtained before the initiation of an ani-bacterial therapy, and at least one of the samples may be obtained after the initiation of such therapy. To ensure more reliable results and reduce statistical deviations to a minimum, averaging the calculated rates of several sample pairs is preferable. A calculated or average value of a negative rate of change in bacterial load, as reflected by the amount of the T3SS component (e.g., EspB) in the sample, indicates that the subject exhibits a beneficial response to the treatment; thereby monitoring the efficacy of a treatment.

The number of samples collected and used for evaluation of the subject may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more.

Thus, by providing a diagnostic tool, the present disclosure further provides therapeutic methods involving a diagnostic step. The diagnostic steps therefore provide tailor made methods allowing monitoring the patient for the presence of the T3S pathogen, during the treatment.

Thus, a further aspect of the present disclosure relates to a method of treating, preventing, ameliorating, reducing or delaying the onset of an infection by at least one bacterium expressing at least one T3SS in a subject in need thereof. In more specific embodiments, the method comprising:

In step (a), classifying a subject as infected by the bacteria if the presence of at least one T3SS component is determined in at least one sample of the subject. In some embodiments, determination of the presence of the at least one T3SS component in the sample is performed by, and/or comprising the following steps:
contacting the at least one sample of the subject with at least one working electrode, at least one reference electrode, and at least one counter electrode, or any biosensor chip device or kit comprising these electroeds. In some embodiments, at least one working electrode is connected directly or indirectly to at least one target binding site and/or moiety; determining electrical voltages between the at least one working electrode and the at least one reference electrode in response to electric currents of selected one or more different frequencies applied by the at least one counter electrode;
determining electrical impedances based on a relation between the measured electrical voltage and the electric currents applied at the different frequencies;
determining a charge transfer electrical resistance based on the determined impedances; and
determining presence of the bacterium expressing at least one T3SS in said sample whenever said charge transfer electrical resistance is greater than a predetermined threshold value, thereby classifying said subject as infected by the bacteria.

The next step (b), involves administering to a subject classified as an infected subject in step (a), a therapeutically effective amount of at least one anti-bacterial agent.

In some embodiments, the determination of the presence of the at least one T3SS component in the sample is performed by the method as defined by the present invention.

In yet some other embodiments, the method of the invention may further comprise the step of administering to the subject detected as infected by at least one T3S expressing bacteria, a therapeutically effect amount of at least one anti-bacterial agent.

In some embodiments, the antibacterial agent may be at least one antibiotic agent or any combinations thereof.

In some specific embodiments, the combined diagnostic and therapeutic methods of the invention may be applicable for infections caused by MDR bacteria.

In some more specific embodiments, the bacteria referred to herein may be a gram-negative bacteria.

In yet some other embodiments, the bacteria may be at least one of EPEC and EHEC.

In other specific embodiments, the methods of the invention are applicable for infectious caused by Enteropathogenic Escherichia coli (EPEC).

In some embodiments, the infections relevant to the method of the invention may be associated with at least one of transient enteritis or colitis, cholecystitis, bacteremia, cholangitis, UTI, traveler's diarrhea, neonatal meningitis and pneumonia, or any condition, symptoms or effects associated therewith, as disclosed herein above.

As indicated herein, the invention provides therapeutic methods involving a diagnostic step using the diagnostic biosensor chip device, kits and methods of the present disclosure. A subject diagnosed as infected with at least one T3SS expressing bacteria is treated according to some embodiments of the invention with at least one anti-bacterial agent. It should be further noted that the present disclosure further provides kits comprising the diagnostic biosensor chip device and any associated reagents and kits thereof, and in addition, at least one therapeutic agent, for example, an anti-bacterial compound.

Such agents may include anti-bacterial agent, anti-fungal agent, growth factors, anti-inflammatory agents, vasopressor agents including but not limited to nitric oxide and calcium channel blockers, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGFs), IGF binding proteins (IGFBPs), epidermal growth factor (EGF), platelet derived growth factor (PDGF), neu differentiation factor (NDF), hepatocyte growth factor (HGF), vascular endothelial growth factor (VEGF), heparin-binding EGF (HBEGF), thrombospondins, von Willebrand Factor-C, heparin and heparin sulfates, and hyaluronic acid.

The term “antimicrobial agent” as used herein refers to any entity with antimicrobial activity (either bactericidal or bacteriostatic), i.e. the ability to inhibit the growth and/or kill bacterium, for example Gram negative bacteria. An antimicrobial agent may be any agent which results in inhibition of growth or reduction of viability of a bacteria by at least about 10%, 20%, 30% or at least about 40%, or at least about 50% or at least about 60% or at least about 70% or more than 70%, for example, 75%, 80%, 85%, 90%, 95%, 100% or any integer between 30% and 70% or more, as compared to in the absence of the antimicrobial agent. Stated another way, an antimicrobial agent is any agent which reduces a population of microbial cells, such as bacteria by at least about 30% or at least about 40%, or at least about 50% or at least about 60% or at least about 70% or more than 70%, or any integer between 30% and 70% as compared to in the absence of the antimicrobial agent. In one embodiment, an antimicrobial agent is an agent which specifically targets a bacteria cell. In another embodiment, an antimicrobial agent modifies (i.e. inhibits or activates or increases) a pathway which is specifically expressed in bacterial cells. An antimicrobial agent can include any chemical, peptide (i.e. an antimicrobial peptide), peptidomimetic, entity or moiety, or analogues of hybrids thereof, including without limitation synthetic and naturally occurring non-proteinaceous entities. In some embodiments, an antimicrobial agent is a small molecule having a chemical moiety. For example, chemical moieties include unsubstituted or substituted alkyl, aromatic or heterocyclyl moieties including macrolides, leptomycins and related natural products or analogues thereof. Antimicrobial agents can be any entity known to have a desired activity and/or property, or can be selected from a library of diverse compounds.

In yet some further embodiments, such antibacterial agents may be antibiotic agents. Still further, in some embodiments such antibiotic agent may be at least one beta-lactam antibiotic agent.

The term “β-lactam” or “β-lactam antibiotics” as used herein refers to any antibiotic agent which contains a b-lactam ring in its molecular structure. β-lactam antibiotics are a broad group of antibiotics that include different classes such as natural and semi-synthetic penicillins, clavulanic acid, carbapenems, penicillin derivatives (penams), cephalosporins (cephems), cephamycins and monobactams, that is, any antibiotic agent that contains a β-lactam ring in its molecular structure. They are the most widely-used group of antibiotics. While not true antibiotics, the β-lactamase inhibitors are often included in this group.

β-lactam antibiotics are analogues of D-alanyl-D-alanine the terminal amino acid residues on the precursor NAM/NAG-peptide subunits of the nascent peptidoglycan layer. The structural similarity between β-lactam antibiotics and D-alanyl-D-alanine prevents the final crosslinking (transpeptidation) of the nascent peptidoglycan layer, disrupting cell wall synthesis. Under normal circumstances peptidoglycan precursors signal a reorganization of the bacterial cell wall and, as a consequence, trigger the activation of autolytic cell wall hydrolases. Inhibition of cross-linkage by β-lactams causes a buildup of peptidoglycan precursors, which triggers the digestion of existing peptidoglycan by autolytic hydrolases without the production of new peptidoglycan. As a result, the bactericidal action of β-lactam antibiotics is further enhanced. Generally, β-lactams are classified and grouped according to their core ring structures, where each group may be divided to different categories. The term “penam” is used to describe the core skeleton of a member of a penicillin antibiotic. i.e. a β-lactam containing a thiazolidine rings. Penicillins contain a β-lactam ring fused to a 5-membered ring, where one of the atoms in the ring is sulfur and the ring is fully saturated. Penicillins may include narrow spectrum penicillins, such as benzathine penicillin, benzylpenicillin (penicillin G), phenoxymethylpenicillin (penicillin V), procaine penicillin and oxacillin. Narrow spectrum penicillinase-resistant penicillins include methicillin, dicloxacillin and flucloxacillin. The narrow spectrum β-lactamase-resistant penicillins may include temocillin. The moderate spectrum penicillins include for example, amoxicillin and ampicillin. The broad spectrum penicillins include the co-amoxiclav (amoxicillin+clavulanic acid). Finally, the penicillin group also includes the extended spectrum penicillins, for example, azlocillin, carbenicillin, ticarcillin, mezlocillin and piperacillin. Other members of this class include pivampicillin, hetacillin, bacampicillin, metampicillin, talampicillin, epicillin, carbenicillin, carindacillin, tie arcillin, azlocillin, piperacillin, mezlocillin, mecillinam, pivmecillinam, sulbenicillin, clometocillin, procaine benzylpenicillin, azidocillin, penamecillin, propicillin, pheneticillin, cloxacillin and nafcillin.

β-lactams containing pyrrolidine rings are named carbapenams. A carbapenam is a β-lactam compound that is a saturated carbapenem. They exist primarily as biosynthetic intermediates on the way to the carbapenem antibiotics. Carbapenems have a structure that renders them highly resistant to β-lactamases and therefore are considered as the broadest spectrum of β-lactam antibiotics. The carbapenems are structurally very similar to the penicillins, but the sulfur atom in position 1 of the structure has been replaced with a carbon atom, and hence the name of the group, the carbapenems. Carbapenem antibiotics were originally developed from thienamycin, a naturally-derived product of Streptomyces cattleya. The carbapenems group includes: biapenem, doripenem, ertapenem, imipenem, meropenem, panipenem and PZ-601. β-lactams containing 2, 3-dihydrothiazole rings are named penems. Penems are similar in structure to carbapenems. However, where penems have a sulfur, carbapenems have another carbon. There are no naturally occurring penems; all of them are synthetically made. An example for penems is faropenem.

β-lactams containing 3, 6-dihydro-2H-1, 3-thiazine rings are named cephems. Cephems are a subgroup of b-lactam antibiotics and include cephalosporins and cephamycins. The cephalosporins are broad-spectrum, semisynthetic antibiotics, which share a nucleus of 7-aminocephalosporanic acid. First generation cephalosporins, also considered as the moderate spectrum includes cephalexin, cephalothin and cefazolin. Second generation cephalosporins that are considered as having moderate spectrum with anti-Haemophilus activity may include cefaclor, cefuroxime and cefamandole. Second generation cephamycins that exhibit moderate spectrum with anti-anaerobic activity include cefotetan and cefoxitin. Third generation cephalosporins considered as having broad spectrum of activity includes cefotaxime and cefpodoxime. Finally, the fourth generation cephalosporins considered as broad spectrum with enhanced activity against Gram positive bacteria and β-lactamase stability include the cefepime and cefpirome. The cephalosporin class may further include: cefadroxil, cefixime, cefprozil, cephalexin, cephalothin, cefuroxime, cefamandole, cefepime and cefpirome. Cephamycins are very similar to cephalosporins and are sometimes classified as cephalosporins. Like cephalosporins, cephamycins are based upon the cephem nucleus. Cephamycins were originally produced by Streptomyces, but synthetic ones have been produced as well. Cephamycins possess a methoxy group at the 7-alpha position and include: cefoxitin, cefotetan, cefmetazole and flomoxef.

β-lactams containing 1, 2, 3, 4-tetrahydropyridine rings are named carbacephems. Carbacephems are synthetically made antibiotics, based on the structure of cephalosporin, a cephem. Carbacephems are similar to cephems but with a carbon substituted for the sulfur. An example of carbacephems is loracarbef. Monobactams are b-lactam compounds wherein the β-lactam ring is alone and not fused to another ring (in contrast to most other β-lactams, which have two rings). They work only against Gram negative bacteria. Other examples of monobactams are tigemonam, nocardicin A and tabtoxin.

β-lactams containing 3, 6-dihydro-2H-I, 3-oxazine rings are named oxacephems or clavams. Oxacephems are molecules similar to cephems, but with oxygen substituting for the sulfur. Thus, they are also known as oxapenams. An example for oxapenams is clavulanic acid. They are synthetically made compounds and have not been discovered in nature. Other examples of oxacephems include moxalactam and flomoxef. Another group of β-lactam antibiotics is the β-lactamase inhibitors, for example, clavulanic acid. Although they exhibit negligible antimicrobial activity, they contain the β-lactam ring. Their sole purpose is to prevent the inactivation of β-lactam antibiotics by binding the β-lactamases, and, as such, they are co-administered with β-lactam antibiotics. β-lactamase inhibitors in clinical use include clavulanic acid and its potassium salt (usually combined with amoxicillin or ticarcillin), sulbactam and tazobactam.

It should be appreciated that the present disclosure may further provides a diagnostic-therapeutic kit. Thus, in some embodiments, the biosensor device of the present invention may be provided in a kit together with at least one anti-bacterial agent (e.g. an antibiotic agent) that may provide means for the combined diagnostic and therapeutic method encompassed by the invention. The kit of the present invention may, if desired, be presented in a pack which may contain one or more units of the kit of the present invention.

The terms “treat, treating, treatment” as used herein and in the claims mean ameliorating one or more clinical indicia of disease activity by administering a pharmaceutical composition of the invention in a patient having a pathologic disorder.

The term “treatment” as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The term “prevention” as used herein, includes the prevention or postponement of development of the disease, prevention or postponement of development of symptoms and/or a reduction in the severity of such symptoms that will or are expected to develop, preventing the occurrence or reoccurrence of the acute disease attacks. These further include ameliorating existing symptoms, preventing-additional symptoms and ameliorating or preventing the underlying metabolic causes of symptoms.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the infectious disease caused by a T3SS expressing MDR bacteria described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of a pathologic disorder or an infectious disease and their symptoms slowing their progress, further exacerbation or development, so as to appear later than in the absence of the treatment according to the invention.

More specifically, treatment or prevention include the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

It should be understood that the therapeutic methods of the invention involve any applicable mode of administration. The phrases “systemic administration”, “administered systemically” as used herein mean the administration of a compound, drug or other material other than directly into the central blood system, such that it enters the patient's system and, thus, is subject to metabolism and other like processes. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

Systemic administration includes parenteral injection by intravenous bolus injection, by intravenous infusion, by sub-cutaneous, intramuscular, intraperitoneal injections or by suppositories, by patches, or by any other clinically accepted method, including tablets, pills, lozenges, pastilles, capsules, drinkable preparations, ointment, cream, paste, encapsulated gel, patches, boluses, or sprayable aerosol or vapors containing these complexes and combinations thereof, when applied in an acceptable carrier. Alternatively, to any pulmonary delivery as by oral inhalation such as by using liquid nebulizers, aerosol-based metered dose inhalers (MDI's), or dry powder dispersion devices.

By “topical administration” it is meant that the therapeutic methods disclosed herein may be adapted to any mode of topical administration including: epicutaneous, transdermal, oral, bronchoalveolar lavage, ophtalmic administration, enema, nasal administration, administration to the ear, administration by inhalation.

The invention provides methods for treating infectious diseases caused by bacterial infections. As used herein, “disease”, “disorder”, “condition” and the like, as they relate to a subject's health, are used interchangeably and have meanings ascribed to each and all of such terms.

It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder, condition or pathology causes a second disease, disorder, condition or pathology.

By “patient”, “individual” or “subject” it is meant any organism who may be affected by the above-mentioned conditions, and to whom the prognostic methods herein described are desired, including humans. More specifically, in some embodiments, the biosensor chip device, kits and methods disclosed herein, are applicable for any mammalian subject. By “mammalian subject” is meant any mammal for which the proposed therapy is desired, including human, equine, canine, and feline subjects, most specifically humans.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

### EXAMPLES

### Experimental Procedures

### Bacterial Strains

Wild-type (WT) EPEC O127:H6 strain E2348/69 [streptomycin-resistant] and EPEC null mutants (ΔescN, ΔespB, ΔespD) were used to purify EspB, to evaluate mAb-EspB-B7 binding, and to assess T3SS and translocation activities[8]. WT and T3SS-mutant strains of Citrobacter rodentium DBS100, enterohemorrhagic E. coli (EHEC), and Salmonella enterica serovar Typhimurium were used to assess antibody specificity. Antibiotics were used at the following concentrations: streptomycin (50 μg/mL), ampicillin (100 μg/mL), chloramphenicol (30 μg/mL), and nalidixic acid (50 μg/mL).

### Chemicals, Antibodies, and Other Reagents

Microcystin-LR and the primary monoclonal antibody (MC10E7) were purchased from Enzo Life Sciences (New York, USA). Potassium hexacyanoferrate (III)/(II) (K3[Fe(CN)6]/K4[Fe(CN)6]) were obtained from Merck (Darmstadt, Germany). All solutions were prepared with ultrapure water obtained from a Milli-Q water purifying system (≥18.2 MΩ cm-1, Millipore). The washing buffer solution was 0.01 M phosphate-buffered saline (1×PBS, pH 7.4) containing 0.1M KCl.

### Expression and Purification of Recombinant EspB

EPEC O127:H6 strain E2348/69 deleted for the espB gene (ΔespB) [8] was transformed with a bacterial expression vector encoding His-tagged EspB (EspB-His) and grown overnight in Luria-Bertani (LB) broth supplemented with the appropriate antibiotics. The following steps of the expression and purification of EspB are described herein. More specifically, the overnight culture was diluted 1:50 and grown for 3 hr under T3SS-inducing conditions (pre-heated Dulbecco's modified Eagle's medium [DMEM] in a tissue culture incubator with 5% CO2, statically). These conditions induce the secretion of EspB into the extracellular environment.

Next, 0.5 mM isopropyl-β-d-thiogalactopyranoside (IPTG) was added, and the culture was grown for an additional 4 hr. The culture was centrifuged for 30 min at 12000×g, and the supernatant containing the secreted EspB-His was collected and supplemented with protease inhibitor cocktail of 200 mM phenylmethylsulfonyl fluoride (PMSF) and 1 μM benzamidine. The supernatant was then loaded on a His-Trap HP 1-mL column (GE Healthcare), washed with 50 mM imidazole, and eluted with 500 mM imidazole, according to the manufacturer's protocol. The elution fractions were analyzed by SDS-PAGE and Coomassie staining to identify the fractions that contain the purified protein. The recovered protein was further purified by gel filtration chromatography using a Superose 12 10/300 GL column (GE Healthcare). The peak fractions were collected, frozen in liquid nitrogen and stored at −80° C.

Phage Panning, mAb-EspB-B7 Expression and Purification

A human synthetic-phage library, displaying single-chain variable fragment (scFv), was used to isolate antibodies targeting EspB as described previously [9]. mAb-EspB-B7 V_{H} and V_{L} were cloned in mammalian expression vectors (pcDNA3.4H and pcDNA3.4L encoding the IgG1 heavy and lambda light chain constant regions) by Gibson cloning. The cloned vectors were transformed into E. coli competent cells (XL-1 blue) and were purified using plasmid purification kit (Invitrogen). The vectors were co-transfected into Expi293 expression system (Gibco) according to the manufacturer's instructions. Transfected Expi293 cells were harvested by centrifugation at 2000×g for 10 min at 4° C. and conditioned medium was applied to MabSelect affinity column (GE Healthcare) according to the manufacturer's instructions.

### Enzyme-Linked Immunosorbent Assay (ELISA)

For all ELISA experiments, 96-well ELISA plates were coated with 5 μg/mL of target antigen in PBS and incubated overnight at 4° C. Blocking, washing and detection steps were carried out as described [10] previously. More specifically, EspB coated 96-well plates were blocked with 300 μL/well of 3% [w/v] skim milk in PBS for 1 hr at 37° C. and washed with PBS. mAb-EspB-B7 in blocking solution was added to the first line of the plate and serially diluted throughout the plate. The plate was incubated for 1 hr at room temperature, washed, and incubated with goat anti-human H+L HRP-conjugated secondary antibody in 0.05% PBST (Jackson ImmunoResearch) for 1 hr at room temperature. Plates were then washed and signal was developed using 3,3′,5,5′-tetramethylbenzidine (TMB). The reactions were quenched by 1 M H₂SO₄ and absorbance was measured at optical density (OD) of 450 nm (Epoch, BioTek).

ELISA assays to test mAb-EspB-B7 binding in various conditions were carried out using similar protocol as described above with the following modifications: (i) for binding under various pH conditions, mAb-EspB-B7 was incubated in 0.1 M citric acid buffer pH 7.4, 7.0, 6.6, 5.6, and 4.6 during the binding step; (ii) for binding at various salt concentrations, mAb-EspB-B7 was incubated in 45.6 nM, 68.5 nM, 137 nM, 274 nM, and 411 nM NaCl; and (iii) for assessment of the serum effect on mAb-EspB-B7 binding, the antibody was incubated in 10% goat or horse serum with 1% Tween 20 and 1% human serum during the binding step. Competitive ELISA with peptides was carried out as follows: A 96-well ELISA plate (I) and a 96-well inert Bradford plate (II) were used for each of the peptides examined. The respective scrambled peptides (carrying the same amino acid compositions in a scrambled order), full-length EspB and peptide #78 (SEQ ID NO. 38) were used as positive and negative controls, respectively. Plate I was coated with 3 μg/ml EspB or PBS and incubated overnight at 4° C. Blocking of plate I was performed as described above. mAb-EspB-B7 (15 nM) was pre-incubated with serially diluted concentrations of peptides, starting at 15 μg/mL for 1 hr at room temperature, transferred to plate I, and incubated for 1 hr at room temperature. The remaining steps were performed as described above for regular ELISA.

### Surface Plasmon Resonance (SPR)

Association and dissociation of the EspB-mAb-EspB-B7 complex was monitored by SPR with a Biacore 200 apparatus (GE Healthcare Life Sciences) with EspB immobilized on a CM5 chip (GE Healthcare Life Sciences). SPR experiments were conducted according to the manufacturer protocols. Immobilization of EspB on CM5 chip was carried out by amine coupling chemistry using the following protocol at a flow rate of 10 μL/min and with 20 mM phosphate buffer with 0.15 M NaCl, and 0.005% Tween 20 at pH 5.91 as a running buffer. The chip was first activated by injecting a freshly prepared mixture of 50 mM N-hydroxysuccinimide and 195 mM 1-ethyl-3-(3-dimethylaminopropyl) carbodiimide for 7.5 min, then EspB (2.5 μg/mL in PBS buffer containing surfactant P20, 10 mM HEPES pH 7.4, 150 mM NaCl, and 3 mM EDTA) was injected for 5 min to reach 120 resonance units (RU), and finally the remaining activated carboxylic groups were blocked by injecting 1 M ethanolamine hydrochloride, pH 8.6, for 5 min. The association of mAb-EspB-B7 with EspB was monitored by injecting different concentrations of mAb-EspB-B7 for 4 min at a flow rate of 30 μL/min, and the dissociation was monitored at the end of the antibody injection. To regenerate the chip, 5 mM NaOH solution was used. Data analysis was carried out by fitting the sensorgrams to the steady state model (T200 evaluation software).

### In Vitro Type 3 Secretion Assay

In vitro T3SS assay was carried out as described previously for EPEC and Salmonella.

EPEC strains were grown overnight in LB supplemented with the appropriate antibiotics in a shaker at 37° C. The cultures were diluted 1:40 into pre-heated DMEM (Biological Industries) and grown statically for 6 hr in a tissue culture incubator (with 5% CO2), to an OD of 0.7 at 600 nm (OD600). These conditions simulate host environment and induce T3SS expression. The cultures were then centrifuged at 20000×g for 5 min to separate the bacterial pellets from the supernatants; the pellets were dissolved in SDS-PAGE sample buffer, and the supernatants were collected and filtered through a 0.22-μm filter (Millipore). The supernatants were then precipitated with 10% (v/v) trichloroacetic acid (TCA) overnight at 4° C. to concentrate proteins secreted into the culture medium. The volume of the supernatants was normalized to the bacterial cultures at OD600 to ensure equal loading of the samples. The samples were then centrifuged at 18000×g for 30 min at 4° C., the precipitates of the secreted proteins were dissolved in SDS-PAGE sample buffer, and the residual TCA was neutralized with saturated Tris. The T3SS activity of C. rodentium was determined similarly to that described for EPEC. For EHEC, the inventors cultured double the amount of EPEC (8 mL cultures instead of 4 mL) due to lower amounts of secreted proteins of EHEC relative to EPEC.

### Immunoblotting

Samples were subject to immunoblotting as described previously [8]. Samples were subjected to SDS-PAGE and transferred to nitrocellulose membranes (pore size: 0.45 μm, Bio-Rad) or polyvinylidene difluoride (PVDF, Mercury, Millipore). The blots were blocked for 1 hr with 5% (w/v) skim milk-PBST (0.1% Tween in phosphate-buffered saline), incubated with the primary antibody (diluted in 5% skim milk-PBST for 1 hr at room temperature or overnight at 4° C.), washed, and then incubated with the secondary antibody (diluted in 5% skim milk-PBST, for 1 hr at room temperature). Chemi-luminescence was detected with EZ-ECL reagents (Biological Industries). The following primary antibodies were used: mAb-EspB-B7, diluted 1:1000; mouse anti-EspB (a gift from Prof. Finlay, University of British Columbia), diluted 1:1000; mouse anti-His (Pierce), diluted 1:2000; mouse anti-JNK (BD Pharmingen), diluted 1:1000 in TBS; and mouse anti-actin (MPBio), diluted 1:10,000. The following secondary antibodies were used: horseradish peroxidase-conjugated (HRP)-goat anti-mouse (Abcam Inc.) and HRP-conjugated goat anti-human (Abcam Inc) antibodies.

### Flow Cytometry

EPEC bacteria were grown overnight in LB with the appropriate antibiotics. The cultures were diluted 1:40 and grown under T3SS-inducing conditions for 3 hr. Thereafter, 1×10⁷ bacteria were plated in a 96-U shape well plate and centrifuged at 800×g for 5 min, and the supernatants were removed. Bacteria were incubated with primary antibody (mAb-EspB-B7, 1:100) for 1 hr at room temperature, washed with PBS, and stained using Alexa Fluor 488 goat anti-human IgG secondary antibody (Jackson ImmunoResearch) for 30 min. Samples were washed and resuspended in PBS for analysis. Flow cytometry analysis was performed on Gallios (Beckman Coulter) equipped with 488 nm, 405 nm and 638 nm lasers and a switchable 561 nm laser. Data analysis was performed with Kaluza software (Beckman Coulter).

### Co-Elution of EspB and EspD35-his by Nickel Affinity Chromatography

Co-elution assays were performed as previously described [8]. EPEC ΔespD in the presence or the absence of an EspD-³⁵His expression vector, was grown under T3SS-inducing conditions for 7 hr (0.5 mM IPTG was added after 3 hr to induce protein expression). To evaluate the ability of mAb-EspB-B7 to inhibit the interaction between EspB and EspD, 100 or 200 nM of mAb-EspB-B7 were added to EPEC ΔespD expressing EspD-35His sample. The supernatants, containing secreted EspD-³⁵His and EspB, were collected by centrifugation (20000×g for 5 min) and were passed through a 0.45-μm-pore-size filter. Protease inhibitor solution was added to the samples (200 mM PMSF and 1 μM benzamidine), and they were incubated with Ni-NTA resin while being rotated overnight at 4° C. The samples were then loaded on gravity columns, and the flow-through was collected. The columns were washed three times with 5 mL of washing buffer (30 mM phosphate buffer pH 7.5, 500 mM NaCl, 50 mM imidazole), and proteins were eluted using elution buffer (30 mM phosphate buffer pH 7.5, 500 mM NaCl, 500 mM imidazole). Equal volumes of the supernatant and the eluate samples were precipitated with 10% (v/v) TCA for 1 hr at 4° C., centrifuged (30 min, 16000×g, 4° C.), air dried, and dissolved in SDS-PAGE sample buffer. Supernatants and eluted samples were analyzed by SDS-PAGE and western blotting using mouse anti-His and mouse anti-EspB antibodies, to avoid detection of the human mAB-EspB-B7 antibody.

### Epitope Mapping Using Peptide Array

Peptide microarrays of 15-residues cyclic peptides, derived from the EspB sequence and containing an overlap of 11 residues, were obtained from JPT Peptide Technologies GmbH.

Peptide array analysis was carried out according to the manufacturer protocols. Each microarray included three identical subarrays as technical triplicates. Full-length EspB protein was spotted on the array and used as a positive control, while bovine serum albumin (BSA) served as a negative control. The binding of mAb-EspB-B7 to the peptide array was carried out according to the manufacturer's instructions (www.jpt.com), with minor modifications. Briefly, 20 μg/mL mAb-EspB-B7 (0.1% TBST v/v) were incubated on the peptide microarray for 2 hr at room temperature. The peptide microarray slides were then washed (five times with TBST), incubated with Alexa Fluor 647-affinipure mouse anti-human IgG (Jackson ImmunoResearch) for 45 min at room temperature, washed (five times with TBST and then five times with doubly distilled H2O), and dried. Fluorescence was detected with a GenePix 4000B scanner (Molecular Devices) at a resolution of 10 μm pixel size and analyzed by the Genepix Pro 6.0 analysis software (Molecular Devices). Signals were normalized and plotted to reflect the relative intensities of the fluorescence signals.

### Effector Translocation Activity

Translocation assays were performed as previously described. More specifically, HeLa cells (8×10⁵ cells per well) were infected for 3 hr with EPEC strains that were pre-induced for 3 hr for T3SS activity (pre-heated DMEM, statically, in a CO₂ tissue culture incubator). Cells were then washed with PBS, collected, and lysed with RIPA buffer. Samples were centrifuged at 18000×g for 5 min to remove non-lysed cells, and supernatants were collected, mixed with SDS-PAGE sample buffer, and subjected to western blot analysis with anti-JNK and anti-actin antibodies (loading control). Uninfected samples and the ΔescN mutant strain-infected samples were used as negative controls. To evaluate the ability of mAb-EspB-B7 to inhibit EPEC translocation activity, 400 nM of mAb-EspB-B7 were added to a sample infected with WT EPEC.

### Electrochemical Biosensor Fabrication

Electrochemical biochips were fabricated and biofunctionalized as previously reported. More specifically, as shown by FIG. 10, electrochemical biochips were designed as electrochemical cells (cᵢ) with a three-electrode configuration (working electrode e_{w}, counter electrode e_{c} and a reference electrode eᵣ) and microfabricated on a p-doped Si/SiO₂ substrate (13, with 285 nm thermally grown oxide) by a combination of photolithography (to define the electrodes pattern) and sputtering (gold deposition, Ti/Au 10 nm/90 nm). The wafer-scale fabrication yielded 32 chips each comprising three gold electrodes (100 nm Au) as well as contact pads (13w,13r,13c).

The working electrode diameter was 0.6 mm. On-chip Ag/AgCl reference electrodes (eᵣ) were prepared by electroplating (in an electroplating bath), and the individual chips were finally diced. The generated chips were characterized electrochemically and by scanning electron microscopy. The mAbs were thiolated by its incubation with Traut's reagent at a molar ratio of 1:15 for 1 hr at room temperature followed by washing with 0.1M phosphate buffer pH 5 to remove the unreacted reagent. Thiolated mAbs were then covalently immobilized onto the gold working electrodes (e_{w}) of the chips by drop-casting after thoroughly cleaning the electrodes by immersing 20 min in a solution of 50 mM KOH and 25% H₂O₂ followed by thorough rinsing with Milli-Q water.

### Electrochemical Biosensor Fabrication of Anti Cyanotoxin Chip

Electrochemical biochips were designed as electrochemical cells with a three-electrode configuration (working, counter and reference electrodes), microfabricated on a p-doped Si/SiO₂ substrate (with 285 nm thermally grown oxide) by a combination of photolithography (to define the electrodes pattern) and sputtering (gold deposition). The process flow showing the step-by-step fabrication of electrochemical chips is shown in FIG. 17C. The wafer-scale fabrication yielded 31 chips, each comprising three gold electrodes (100 nm thick Au layer) as well as contact pads. The working electrode diameter was 0.6 mm. On-chip Ag/AgCl reference electrodes were prepared in-house by electroplating and the individual chips were finally diced. The generated chips were characterized electrochemically and by scanning electron microscopy, as shown in FIG. 17D and FIG. 19.

### Biosensor Measurements

Biosensor measurements were based on Electrochemical Impedance Spectroscopy (EIS) recorded by a commercial potentiostat device (BioLogic). The faradaic current response of a routinely employed redox couple (10 mM K₃Fe(CN)₆) found within the measurement buffer, was monitored both by cyclic voltammetry (CV) and EIS. The impedance spectra of the freshly cleaned electrodes were obtained prior and post antibody immobilization, with a potential amplitude of 5 mV at a frequency range of 100 kHz to 10 Hz. The CV was collected within a potential range of −200-600 mV vs. Ag/AgCl at a scan rate of 100 mV/sec. Purified EspB protein solutions (at the concentration of 0, 1, 4, 10, and 250 μg/mL in PBS) were incubated for 10 min on the working electrode and then measured by the EIS method. The specificity of the obtained signals was verified by two control experiments. A negative control that included an unrelated antigen (2 μg/ml of the toxin Microcystin-LR, PubChem CID: 445434) and additionally, a purified EspB antigen (2 μg/mL) without the immobilized mAb (on a nonfunctionalized bare electrode). All Measurements of soluble EspB were repeated 3-6 times for each protein concentration. The charge transfer resistance (R_{ct}) values were obtained by fitting the generated Nyquist plots to a Randles equivalent circuit. The percent change in R_{ct} ratios between the biofunctionalized electrodes and varying EspB concentrations was calculated and averaged from: ΣR_{ct}(EspB)/R_{ct}(mAb)−1.

In order to detect whole bacterial cell suspensions, EPEC WT and ΔespB mutant strains were cultured as described herein above, gently centrifuged (500×g, 5 min) and resuspended in PBS to a concentration of 3×10⁷ cells/mL. Five microliters of bacteria-containing samples were incubated on the biochip electrode for 10 min, the electrode was then rinsed and CV and EIS measurements were taken. The percent change in R_{ct} ratios measured for EPEC WT and ΔespB was calculated and averaged from 20 repeats (five measurements each containing four samples) for each strain. The mean of the averaged ratios and the standard error of the mean were calculated. Differences between the means were statistically significant as indicated by a t-test using an alpha level of 0.05. In order to compare the means of R_{ct} ratios of both strains, standard errors were combined in quadrature.

### Nano Differential Scanning Fluorimetry (NanoDSF)

To assess the thermal stability of mAb-B7, 20 μM mAb-B7 samples were loaded into UV capillaries (NanoTemper Technologies) and analyzed using the nanoDSF Prometheus NT.48. The temperature gradient was set to 1° C./min increase between 15° C. and 95° C. The melting temperatures (Tm) that was derived from protein unfolding was presented by plotting the tryptophan fluorescence at λ=330 nm and λ=350 nm over temperature. The melting temperatures were determined by calculating the maximum of the first derivative and the peak position (at Tₘ) was determined.

### Electrochemical Impedance Spectroscopy (EIS) Measurements

Biosensor measurements were based on Electrochemical Impedance Spectroscopy (EIS) recorded by a commercial potentiostat device (BioLogic, Seyssinet-Pariset, France). EIS was employed to examine the gold electrode before and after modification with the thiolated EspB-specific monoclonal antibody. In a faradaic impedance measurement, a small sinusoidal AC voltage probe is applied (S1 in FIG. 15) while monitoring the current response (S2) at different frequencies. The real (resistive) component of the impedance (determined by the in-phase current response) is plotted against the imaginary (capacitive) component (determined by the out-of-phase current response) with respect to frequency (S3). Both are described by

$${Z}^{′} = {R}_{s} + \frac{{R}_{c t}}{1 + {ω}^{2} {R}_{c t}^{2} {C}_{d l}^{2}} and {Z}^{″} = \frac{{R}_{c t}^{2} {C}_{d l} ω}{1 + {ω}^{2} {R}_{c t}^{2} {C}_{d l}^{2}} ,$$

where Rₛ is the solution resistance, R_{ct} the charge transfer resistance, C_{dl} is the double-layer capacitance, and ω the angular frequency, which is commonly represented in a Nyquist plot. The impedance results of the thus-obtained Nyquist plot are fitted (S4) to an equivalent circuit (Randles circuit) used to interpret the electrochemical system, as shown in the inset of FIG. 12A. An increase in charge transfer resistance (R_{ct}) is attributed to surface adsorption of bound biomolecules (S6).

### The Randles Circuit

To extract the parameter of interest, a generated Nyquist plot is commonly fitted to a model equivalent electronic circuit, Randles circuit. If an analyte affects one of these circuit parameters, then impedance methods can be used for analyte detection. The R_{ct} depicts the opposition experienced to electron movement and it increases in the presence of bound biomolecules. For a one-electron process the R_{ct}, which controls the electron transfer kinetics of Fe(CN)₆^{4-/3-} at the interface of the electrode, can be described by:

$${R}_{c t} = \frac{R T}{{F}^{2} {κ}^{2} C}$$

where R denotes the gas constant, T is temperature, F is Faraday constant, κ^{D} is the electron transfer rate constant and C is the concentration of the electroactive species [I. I. Suni, TrAC—Trends Anal. Chem. 27 (2008) 604-611; K. Hara, et al., Theory, Experiment, (2013). https://doi.org/10.1016/j.snb.2007.02.003]. The semi-circular region represents a slower charge transfer at higher frequencies whereas the straight line describes a faster mass transfer at lower frequencies. Also, a change in Warburg impedance, Z_{w}, which is dominated by mass transfer can occur when the diffusional transport of electroactive species from the bulk solution to the electrode surface is impeded due to the binding of biomolecules and targets onto the electrode [J. Yeh, B. et al., Sensors Actuators, B Chem. 237 (2016) 329-340]. However, both R_{ct} and Z_{w} depend on the concentration of electroactive species and the applied potential [A. Lasia, Electrochemical Impedance Spectroscopy and its Applications—Andrzej Lasia—Google Books, (2014)https://books.google.com/books?id=lWEgBAAAQBAJ&printsec=frontcover&source=gbs_ge_summary_r&cad=0#v=onepage&q&f=false].

The Nyquist plots arising from EIS measurements were fitted to the following Randles circuit from which the parameter of interest, R_{ct}, values were calculated.

### Electrochemical Characterization of Chips

The quality of the electroplated Ag/AgCl quasi reference electrode (RE), and of the whole cell were electrochemically characterized. The RE potential demonstrated a linear dependence on the log of the electrolyte (NaCl) concentration, as expected, following the Nernst equation (FIG. 19).

An example of cyclic voltammetry (CV) for the EC biochip is presented in FIG. 20A, where four different scan rates were used consecutively. The peak heights increased with increasing scan rates, and were linearly proportional to the square root of the scan rates (FIG. 20B), as expected, following the Randles-Sevick equation. In addition, the peak separation was not significantly affected by the scan rate (FIG. 20C).

### Biofunctionalization

Antibodies were thiolated and immobilized to the gold working electrodes.

The electrodes were modified via thiol-modification of primary amines (—NH₂) of the anti-MC-LR antibody to introduce sulfhydryl (—SH) groups, which allowed covalent immobilization of the thiolated antibodies to the gold working electrode. Briefly: 14 mM solution of Traut's reagent (2-Iminothiolane, 2-IT) was prepared by dissolving 2 mg in 1 mL PBS. The solution was diluted to 0.14 mM in 1×PBS. MC10E7 antibody stock was diluted to 20 μg/mL (6×10⁻⁸ M) and mixed with the 2-IT reagent at a ratio of 1:15 antibody: reagent). The reaction mixture was incubated for 1 hour at room temperature. Subsequently, free 2-IT was removed by ultrafiltration using Centricons with a MW cutoff of 50 kDa. A volume of 0.5 mL was centrifuged (14,000 g, 15 minutes) and the retentate was resuspended in 0.5 mL of 0.1 M phosphate buffer pH 5. The number of thiol groups per antibody was quantified using Ellman assay.

More specifically, Antibodies were thiolated in order to obtain a firm immobilization via gold-sulfur covalent bond. Traut's reagent (2-Iminothiolane, 2-IT) reacted with antibody primary amines to yield sulfhydryl groups, according to the mechanism shown in FIG. 21A. Estimation of introduced sulfhydryl groups was performed by Ellman assay. Ellman's Reagent (5,5′-dithiobis-(2-nitrobenzoic acid, or DTNB) is used to quantify the number or concentration of thiol groups in a sample. It is very useful as a sulfhydryl assay reagent because of its specificity for —SH groups at neutral pH, high molar extinction coefficient and short reaction time. DTNB reacts with a free sulfhydryl group to yield a mixed disulfide and 2-nitro-5-thiobenzoic acid (TNB). The target of DTNB in this reaction is the conjugate base (R—S⁻) of a free sulfhydryl group. TNB is the “colored” species produced in this reaction and has a high molar extinction coefficient with a value of 14,150M⁻¹cm⁻¹ at 412 nm. The DTNB reduction reaction and its structure are shown in FIG. 21B. Introduced —SH groups were quantified by reference to the extinction coefficient of TNB following:
C=A/bE; where A=absorbance, b=optical path length (cm), E=molar extinction coefficient, and
C=concentration (molar). Antibodies incubated with Traut's reagent at a ratio of 1:10 and 1:15, yielded an average —SH groups per antibody of 3.63 and 6.7, respectively.

Before immobilization, the electrodes were cleaned by immersing 30 minutes in a solution of 50 mM KOH and 25% H₂O₂ followed by thorough rinsing with Milli-Q water. Following this, the thiolated MC10E7 antibodies were covalently immobilized onto the gold working electrode (WE) by drop-casting (3 μl directly on the working electrode) and incubating for 2 hours at 37° C. Assessment of thiolated antibodies immobilization to the gold working electrode was carried out by fluorescence microscopy analysis. Fluorescently (Cy3)-labeled antibodies were used in this assay. The Cy3-labeled antibody was thiolated according to protocol and incubated on the gold WE for different incubation times. As a control, a non-thiolated Cy3-antibody was used. Incubation was followed by rigorous rinsing of the electrodes.

### Electrochemical Measurements

Biosensor measurements were based on Electrochemical Impedance Spectroscopy (EIS). Impedance spectra were recorded before and after electrode functionalization with thiol-modified antibodies, as detailed below (Electrochemical impedance spectroscopy). Quantitative detection was demonstrated using purified MC-LR solutions. Briefly, Purified MC-LR antigen solutions (in PBS supplemented with 0.05% Tween and 0.05% BSA) at the concentrations of 0.0003, 0.003, 0.03, 0.3, 3, and 30 μg/L were incubated for 30 min on the gold working electrode and then measured by the EIS method. Negative control included a non-specific antibody (anti-EspB antibody raised against the virulent EspB protein in pathogenic E. coli [Y. Hillman, et al., Anal. Chem. 93 (2021) 928-935]. All Measurements of soluble MC-LR were repeated three times for each concentration. The change in charge transfer resistance between the biofunctionalized electrodes and varying MC-LR concentrations was calculated from ratios before and after antigen-capture:
ΔR_{CT}=R_{CT}(MCLR)/R_{CT}(Buffer)−1, averaged from triplicates. Additional measurements were performed using cyanobacteria suspensions. To detect MC-LR secreted by cyanobacteria, whole bacterial cell suspensions of Microcystis aeruginosa PPC 7806 (MC-LR-producing cyanobacteria) and Spirulina sp. (non-toxicogenic cyanobacteria) were analyzed by the label-free immunosensor. Briefly, both samples were cultured, grown, and maintained in BG-11 at a temperature of 24-26° C. and light intensity of 6 μmol photons m⁻²s⁻¹. Cell cultures were sub-cultured into a fresh medium 1 week before the experiment. Prior to each experiment, cell counts were performed to determine cell viability and density. The cell concentrations of Microcystis aeruginosa PPC 7806 and Spirulina sp. were 2.08×10⁷ cells/mL and 4.9×10⁷ cells/mL, respectively. The faradaic current response of a routinely employed redox couple (10 mM K₃Fe(CN)₆) found within the measurement buffer (1×PBS pH 7.4 supplemented with 0.1 M KCl), was monitored both by cyclic voltammetry (CV) and EIS. The impedance spectra of the freshly cleaned electrodes were obtained prior and post antibody immobilization, at a frequency range of 10 Hz to 100 kHz, at a potential of 220 mV, using an alternating voltage of 10 mV. The Nyquist plots arising from Electrochemical impedance spectra were fitted to the Randles circuit, shown below, from which the parameter of interest, R_{ct} was calculated.

### Electrochemical Impedance Spectroscopy

Biosensor measurements were based on Electrochemical Impedance Spectroscopy (EIS) recorded by a commercial potentiostat device (BioLogic, Seyssinet-Pariset, France). EIS was employed to examine the gold electrode before and after modification with the thiolated MC-LR-specific monoclonal antibody (MC10E7). In a faradaic impedance measurement, a small sinusoidal AC voltage probe is applied while monitoring the current response at different frequencies. The real (resistive) component of the impedance (determined by the in-phase current response) is plotted against the imaginary (capacitive) component (determined by the out-of-phase current response) to frequency. Both are described by:

$${Z}^{′} = {R}_{s} + \frac{{R}_{c t}}{1 + {ω}^{2} {R}_{c t}^{2} {C}_{d l}^{2}} and {Z}^{″} = \frac{{R}_{c t}^{2} {C}_{d l} ω}{1 + {ω}^{2} {R}_{c t}^{2} {C}_{d l}^{2}} ,$$

where Rₛ is the solution resistance, R_{ct} the charge transfer resistance, C_{dl} is the double-layer capacitance, and ω the angular frequency, which is commonly represented in a Nyquist plot.

### The Randles Circuit

The Nyquist plots arising from EIS measurements were fitted to the following Randles circuit from which the parameter of interest, R_{ct}, values were calculated:

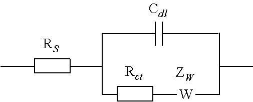

Rₛ—Solution resistance
R_{ct}—Charge transfer resistance
C_{dl}—Double-layer capacitance
Z_{ω}—Warburg Impedance (the angular frequency)

If an analyte affects one of these circuit parameters, then impedance methods can be used for analyte detection. The R_{ct} depicts the opposition experienced to electron movement and it increases in the presence of bound biomolecules. For a one-electron process the R_{ct}, which controls the electron transfer kinetics of Fe(CN)₆^{4-/3-} at the interface of the electrode, can be described by:

$${R}_{c t} = \frac{R T}{{F}^{2} {κ}^{2} C} .$$

where R denotes the gas constant, T is temperature, F is Faraday constant, κ^{D} is the electron transfer rate constant and C is the concentration of the electroactive species [I. I. Suni, Trends Anal. Chem. 27 (2008) 604-611; K. Hara, et al., Theory, Experiment, and, 2013. https://doi.org/10.1016/j.snb.2007.02.003]. The semi-circular region represents a slower charge transfer at higher frequencies whereas the straight line describes a faster mass transfer at lower frequencies. Also, a change in Warburg impedance, Z_{w}, which is dominated by mass transfer can occur when the diffusional transport of electroactive species from the bulk solution to the electrode surface is impeded due to the binding of biomolecules and targets onto the electrode [Yeh, B. et al., Sensors Actuators, B Chem. 237 (2016) 329-340]. However, both R_{ct} and Z_{w} depend on the concentration of electroactive species and the applied potential [A. Lasia, et al., Google Books, 2014].

### MC-LR Detection Based on Indirect Competitive ELISA

The first step is to develop a conventional ELISA, which is required in this study for two reasons: so that it may be properly adjusted to an EC setup, and to be used as a “gold standard”. As shown in FIG. 29 the principle of ic-ELISA for MC detection is based on the recognition of MCs by specific antibodies. Toxins that are present in a sample compete with MCs analogs immobilized on a plate over binding to anti-MC antibodies in solution. The plate is then washed and an HRP-labeled secondary antibody is added. After a second washing step and adding a substrate solution a color signal is generated. The color intensity is inversely proportional to the concentration of MCs present in the sample and is measured using an ELISA reader. The concentrations of the samples are calculated by interpolation using a standard curve. Each calibration point of the standard curve is determined by calculating the mean of the data (n repeats). A standard curve can be obtained by fitting calculated means to Hill equation:

$$a + \frac{( b - a )}{{[ 1 \div ( \frac{c}{X} ) ]}^{d}}$$

Where Ŷ is the expected absorbance at 450 nm at concentration X, a is the minimum asymptote or the absorbance when concentration=0, b=the maximum asymptote or the stabilized absorbance for an infinite concentration, c is the concentration at which 50% of the samples are expected to show the desired absorbance, d is the slope at the steepest part of the curve known as the Hill slope.

### EC Analysis of Enzyme, Substrates, and MC

The enzyme label and substrate used in the ic-ELISA are HRP [Ruzgas et al., Analytica Chimica Acta, 330(2-3), 123-138 (1996)] and TMB (Tetra-methylbenzidine), respectively. In the ECI, however, TMB is substituted with N-acetyl-para-aminophenol (APAP) to avoid electrode fouling. APAP, a common HRP substrate, is not electrochemically active at low potentials. HRP catalyzes APAP oxidation in a pH-dependent manner via a 2-electron and 2-proton process as shown below. This results in N-acetyl para benzoquinone imine (NAPQI) product, which is only stable in its unprotonated form at pH ≥6 [Dahlin et al., Isotopenpraxis, 20(1), 1327-1331 (1984); Kulys et al., Journal of Electroanalytical Chemistry, 321(2), 277-286 (1991)]. NAPQI is electrochemically active enabling its EC determination.

The redox behavior of the substrate APAP and electro-active product NAPQI is analyzed by CV to determine the reduction potential of NAPQI. The redox behavior of the enzyme HRP and the toxin MC-LR are similarly examined.

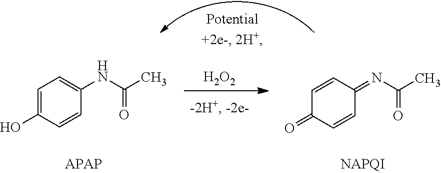

### Development of the Substrate-Mediated Amperometric Immunoassay

The immobilization of MC-LR was based on the formation of self-assembled monolayers (SAM) of the alkanethiol group on gold electrode surfaces. A schematic diagram of the process is illustrated in FIG. 30.

Briefly, the EC chips is washed with successive solutions of ethanol (99.5%), and deionized water (DI) and purged under N₂ gas to remove surface-bound impurities. The gold working electrodes will be preconditioned by drop-casting 1 mM 11-Mercaptoundecanoic acid (11-MUA 95%) prepared in 99.5% ethanol and incubated overnight (16-18 hrs.) at room temperature. Following preconditioning, the 11-MUA modified electrode is rinsed with pure ethanol followed by DI water to remove loosely bound thiol moieties and further dried under a low stream of N₂ gas. For MC-LR binding, the terminal —COOH groups of the 11-MUA-SAM modified electrode will be activated by 0.4 M (3-dimethylaminopropyl)-3-ethylcarbodiimide (EDC) and 0.1 M n-hydroxysuccinimide (NHS) solution for 60 min incubation at room temperature. The EDC-NHS activated electrodes is washed by 1×PBS followed by DI water. 3 μL of 5 μgmL⁻¹ of MC-LR was dropped onto the surface of the MUA/EDC-NHS/GE electrode and incubated for 1 h in the humid chamber to prevent drying of the electrode surface. Then the MC-LR is immobilized covalently by a coupling reaction between the —NH₂ group of the MC-LR and the EDC-NHS-activated MUA moieties on the electrode surface. After MC-LR immobilization the chips (MC-LR/MUA/EDC-NHS/GE) is washed with 1×PBS to remove the loosely bound MC-LR.

Subsequently, the electrodes are washed with 1×PBS buffer containing 0.05% Tween-20 and 0.5% BSA for 2 min to prevent the non-specific binding of the non-targeted protein, thus forming BSA/MC-LR/MUA/EDC-NHS/GE biosensor. Following modification and optimization of the conventional ic-ELISA, it is adapted to the EC system, as illustrated in FIG. 30. This was followed by covering the modified gold electrode (BSA/MC-LR/EDC-NHS/MUA/GE) with 3 μL of mixture of the primary antibody (MC10E7) and HRP-secondary antibody immunoconjugate complexes, incubated at 37° C. for 30 mins. Following a washing step with 1×PBS, three (3) μL of different concentrations of MCs standard solution is immobilized on the 1° Ab-HRP-Ab/BSA/MC-LR/EDC-NHS/MUA/GE modified electrode and incubated for 1 hour at 37° C. MCs bound to free antibodies from the 1° Ab-HRP-Ab mixture bound to the adsorbed MC-LR. Following a washing step with 1×PBS, the HRP substrate (APAP/H₂O₂) is added. Then a potential of −100 mV is applied to allow the enzymatic electro-active product to be reduced on the WE generating measurable current.

### Example 1

Binding Affinity and Specificity o mAb-EspB-B7

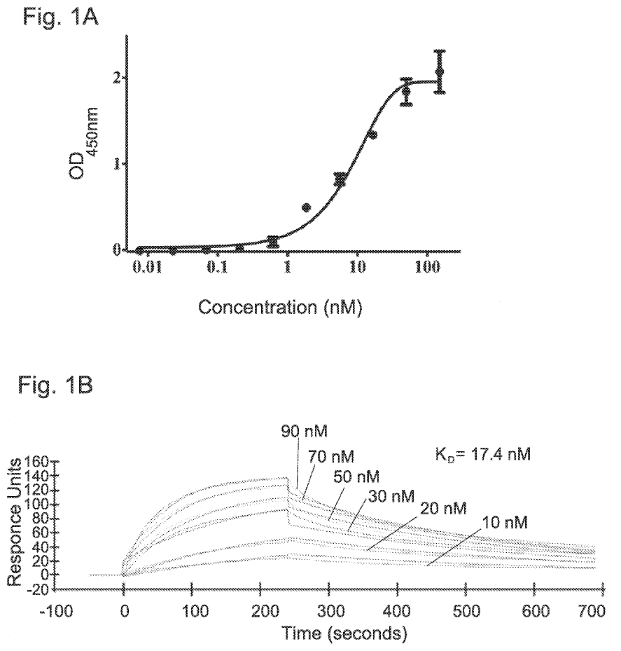

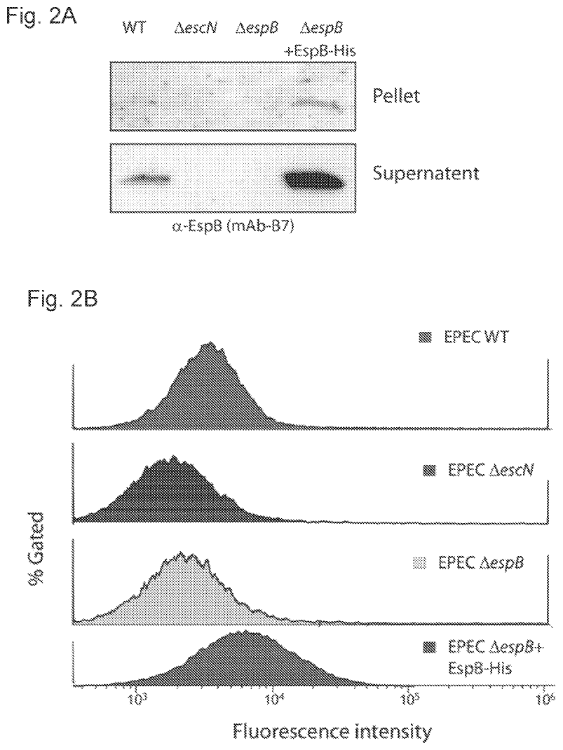

To evaluate the binding affinity of mAb-EspB-B7 to EspB, the inventors performed ELISA (FIG. 1A) and SPR (FIG. 1B) binding assays. It was found that mAb-EspB-B7 binds EspB with high affinity, with a K_{D} value of 17.4 nM. To determine whether mAb-EspB-B7 binds specifically to EspB under native conditions, the binding of mAb-EspB-B7 to WT EPEC, ΔescN (a mutant lacking a functional T3SS), ΔespB (a mutant that does not express and secrete EspB), and ΔespB+EspB-His (an espB null strain that overexpresses plasmid-encoded EspB-His), was examined. The different strains grown under T3SS-inducing conditions were separated into bacterial pellets and supernatants, which were then analyzed by SDS-PAGE and western blot using mAb-EspB-B7 in the detection phase. In agreement with a previous study, a significant secretion of EspB into the bacterial supernatant of WT EPEC was determined, however, not into the supernatants of the ΔescN or ΔespB mutants (FIG. 2A) [Luo, W.; et al., Infect Immun 2006, 74 (2), 810-20]. Moreover, the inventors observed that overexpression of EspB (ΔespB+EspB-His) resulted in a higher expression of the protein within the bacteria (pellet) as well as a higher secretion into the extracellular medium (FIG. 2A).

### Example 2

### Mab-EspB-B7 Binding to EspB in the Assembled T3SS

To examine whether mAb-EspB-B7 can bind to the native protein in the assembled T3SS, flow cytometry was used. For this purpose, the bacterial strains grown under T3SS-inducing conditions were incubated first with mAb-EspB-B7 and then with a secondary antibody conjugated to a fluorophore. As expected, mAb-EspB-B7 binding was detected in WT EPEC and in the ΔespB strain overexpressing EspB, whereas no or minimal binding was detected in the ΔescN and ΔespB mutant strains (FIG. 2B).

### Example 3

Stability of mAb-EspB-B7 Binding Under Various Physiochemical Conditions

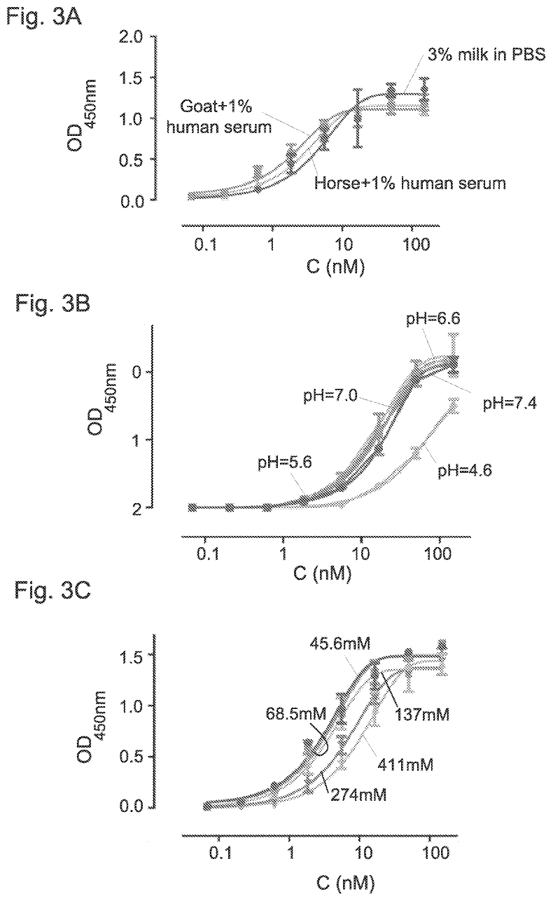

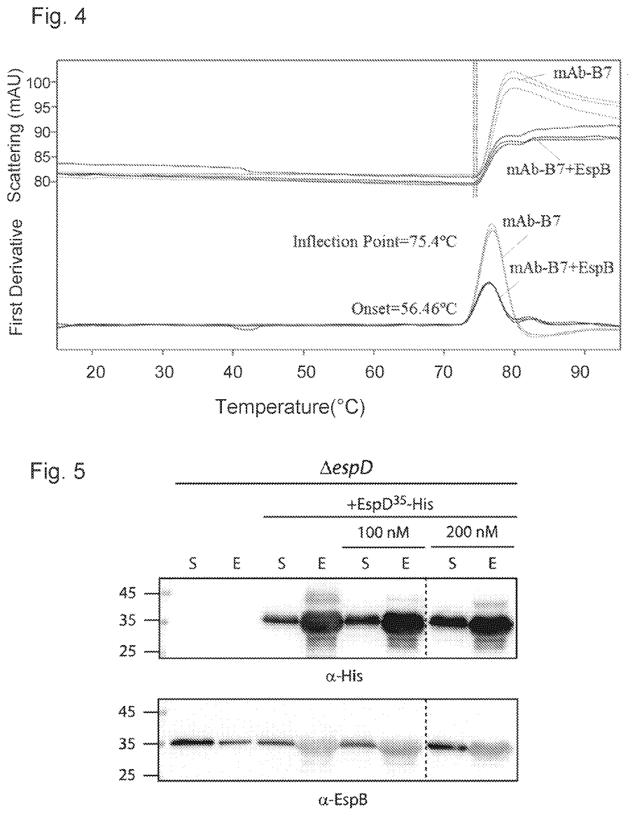

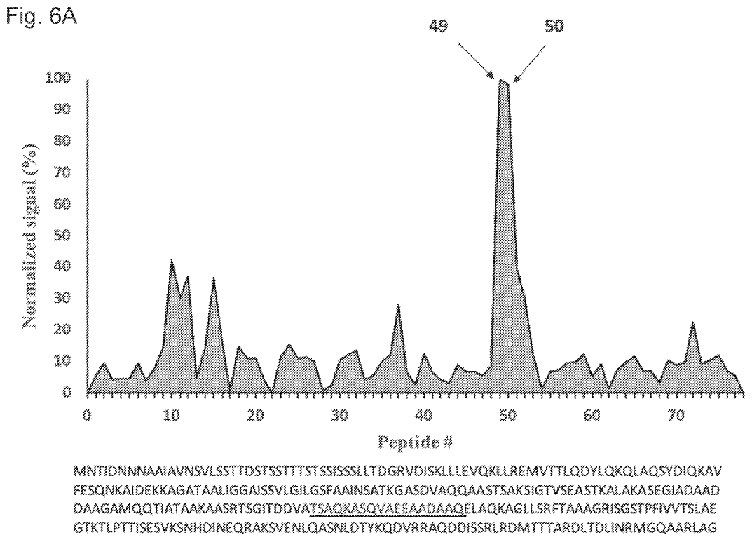

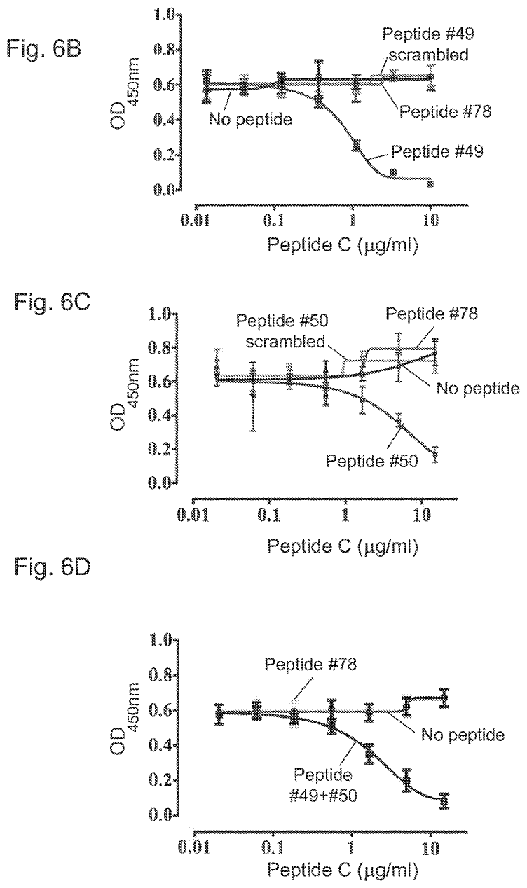

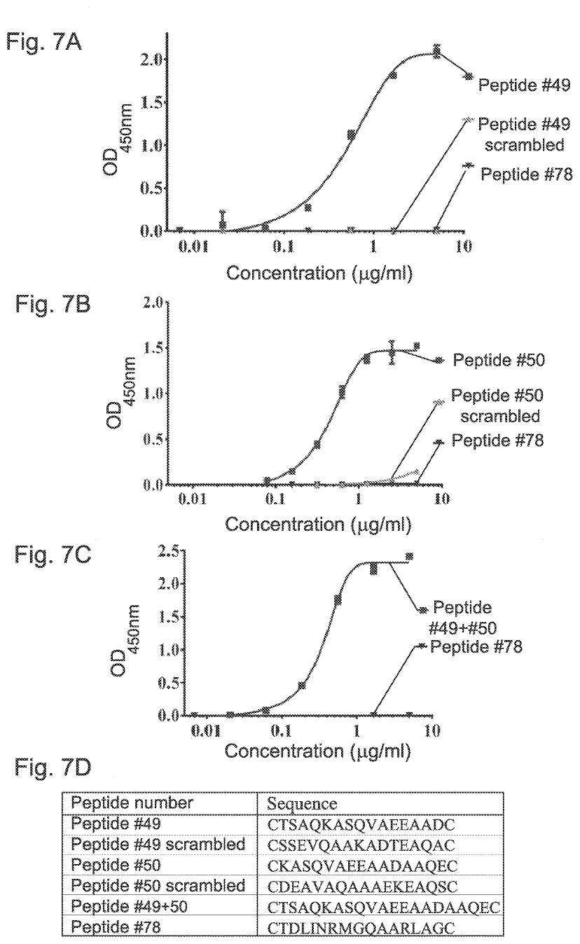

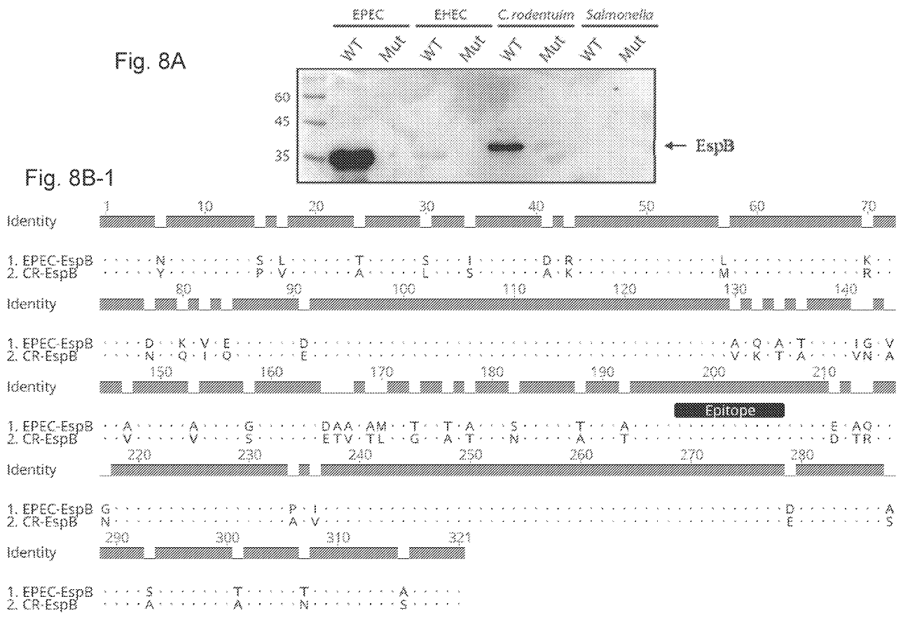

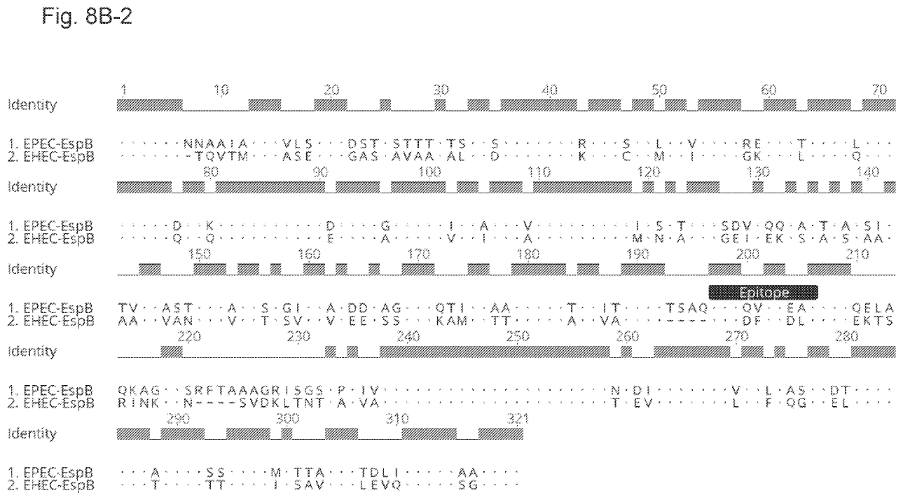

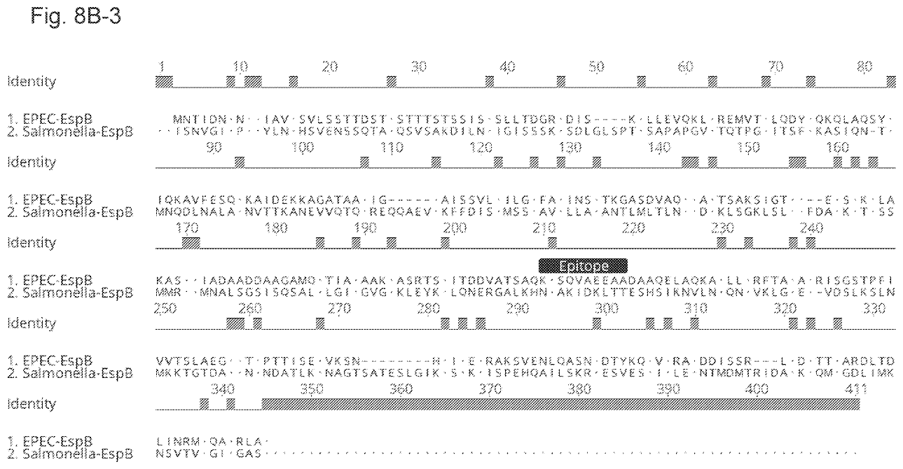

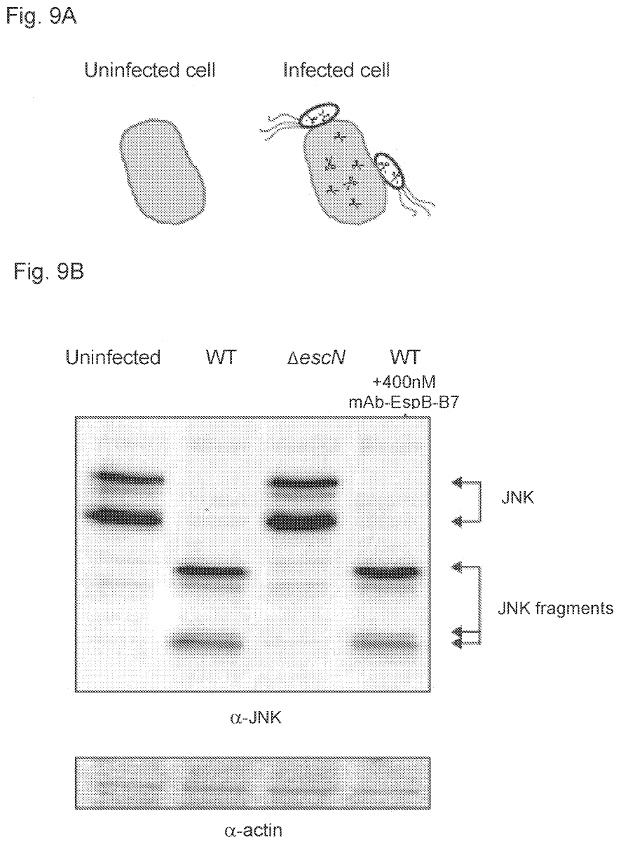

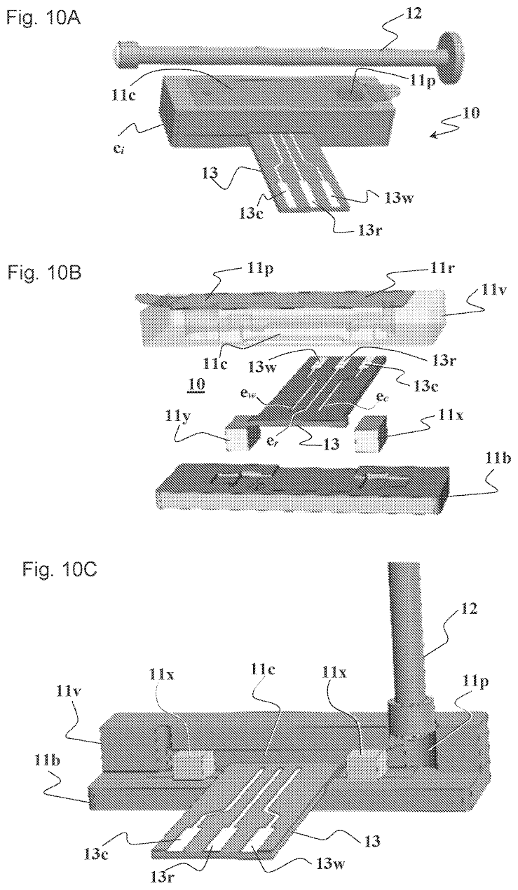

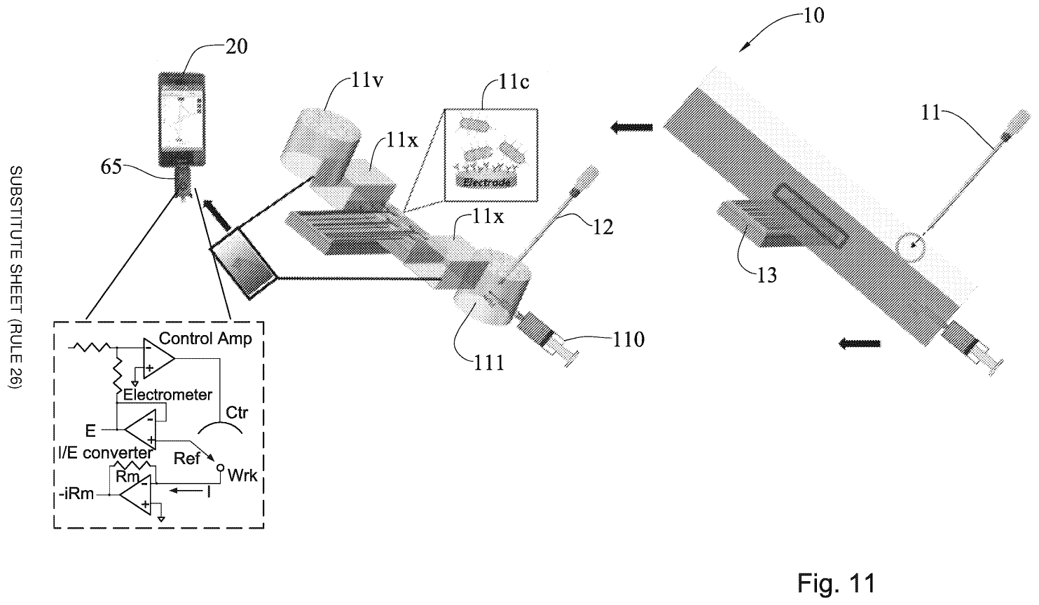

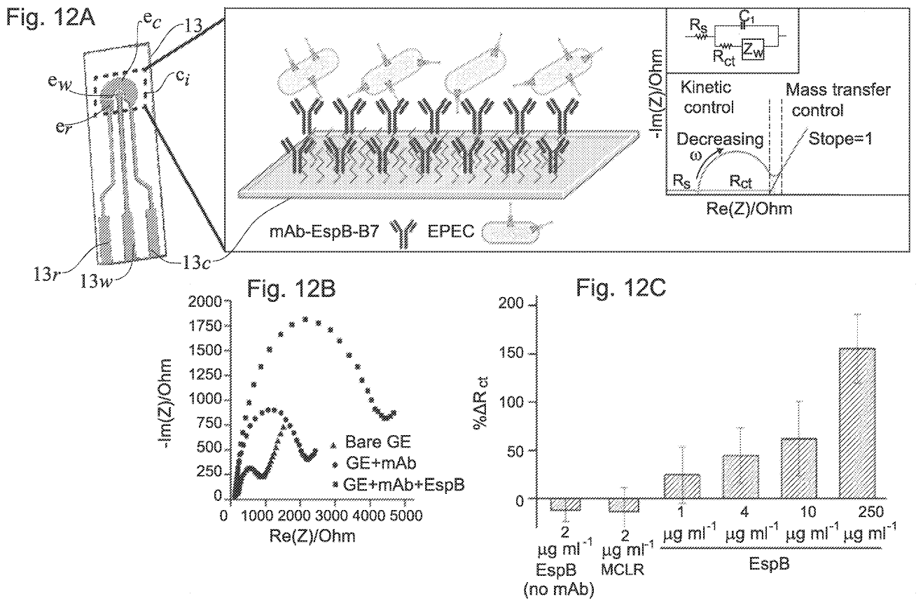

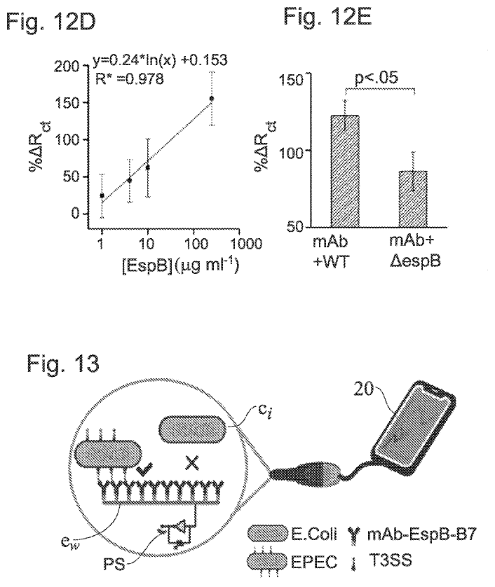

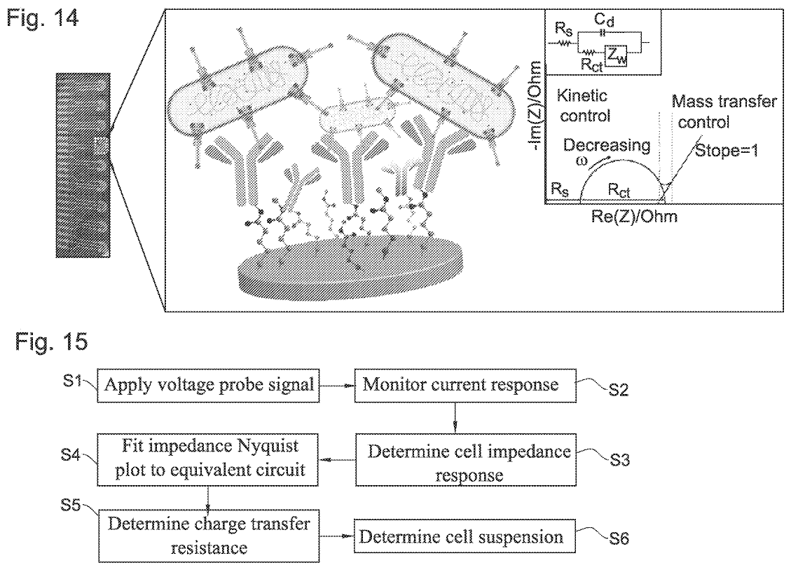

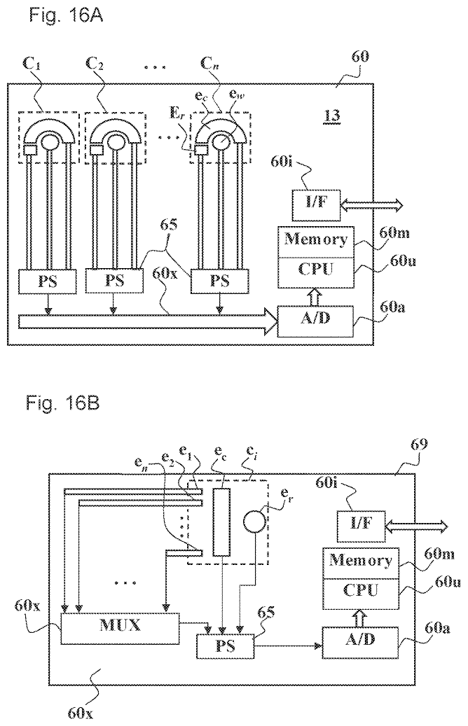

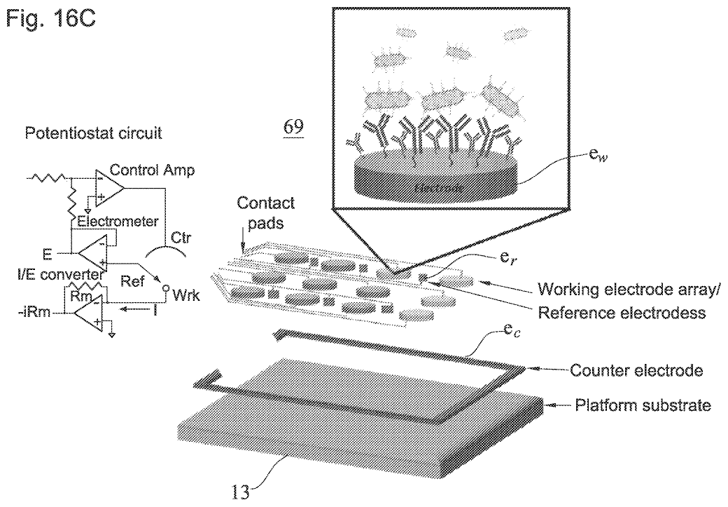

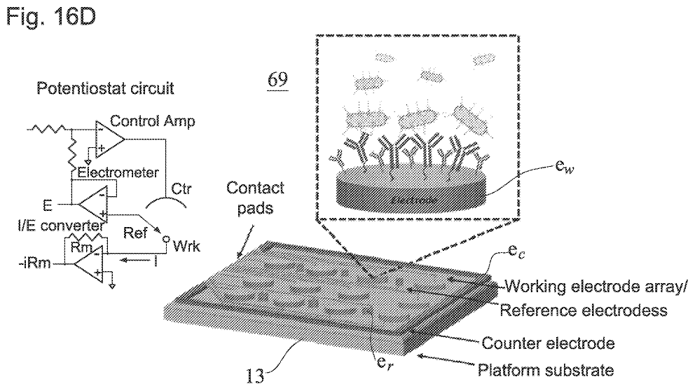

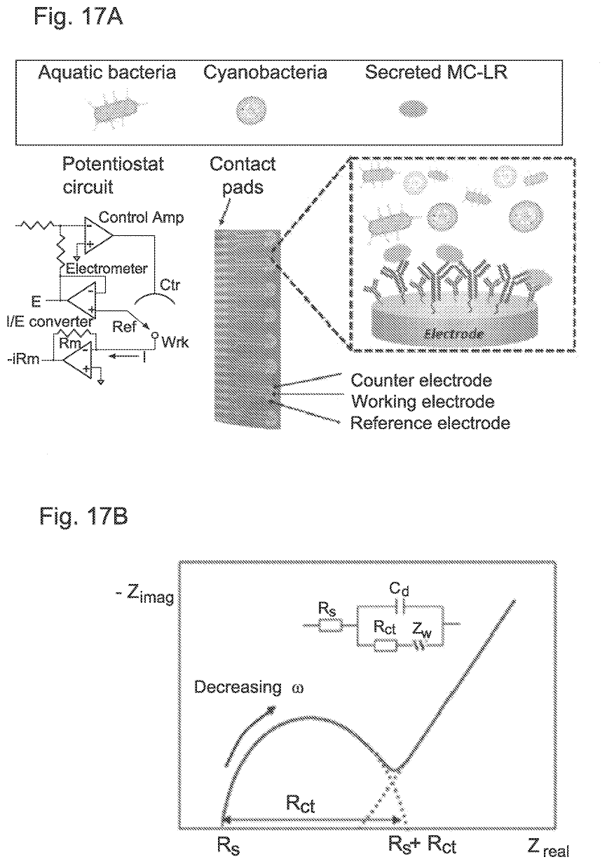

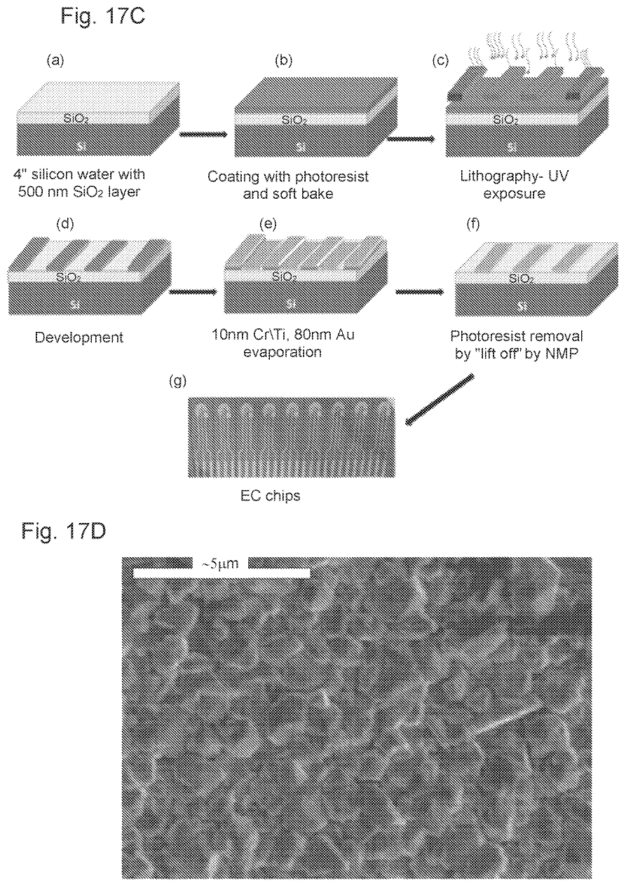

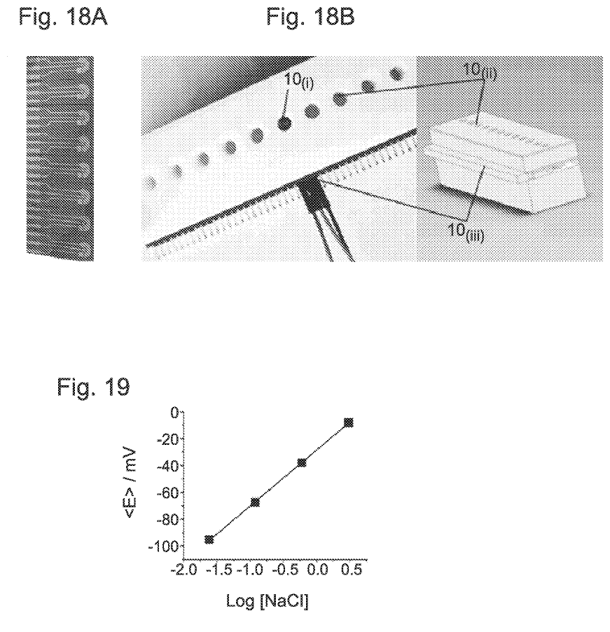

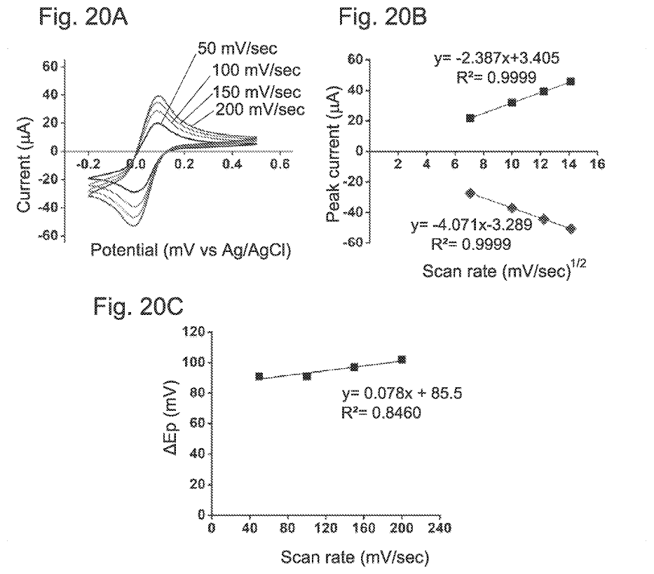

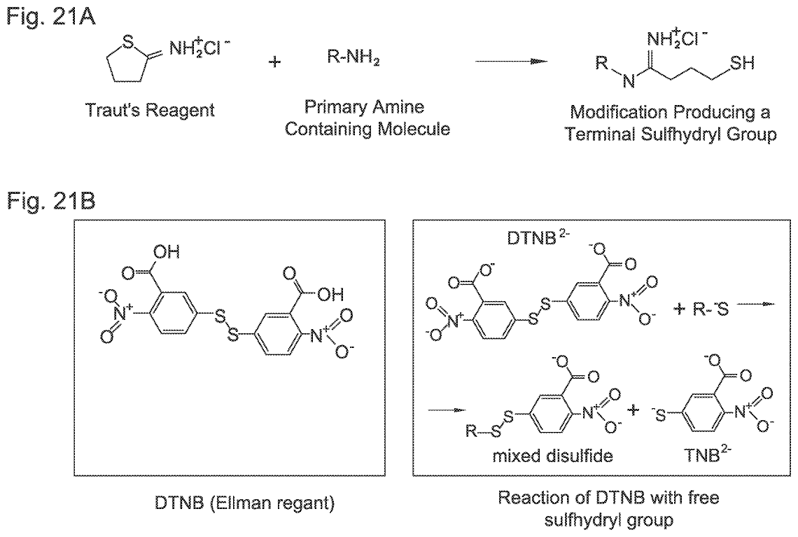

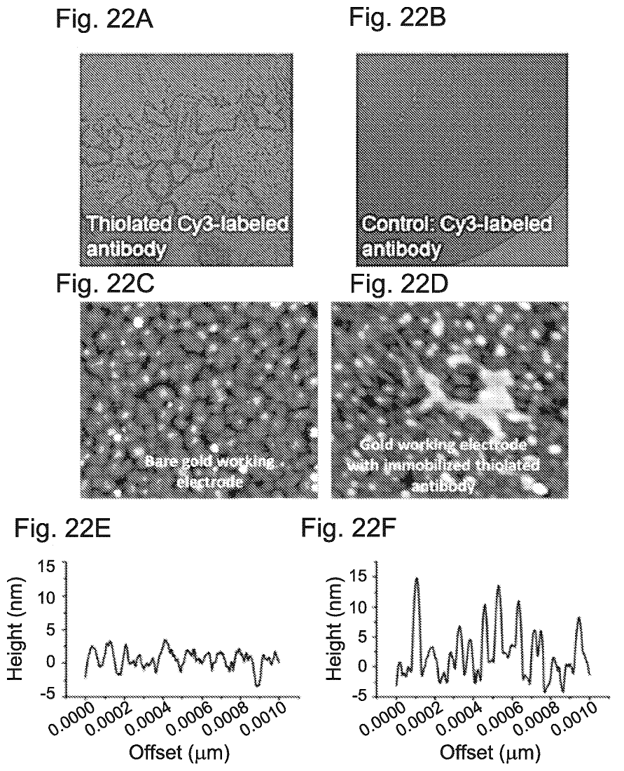

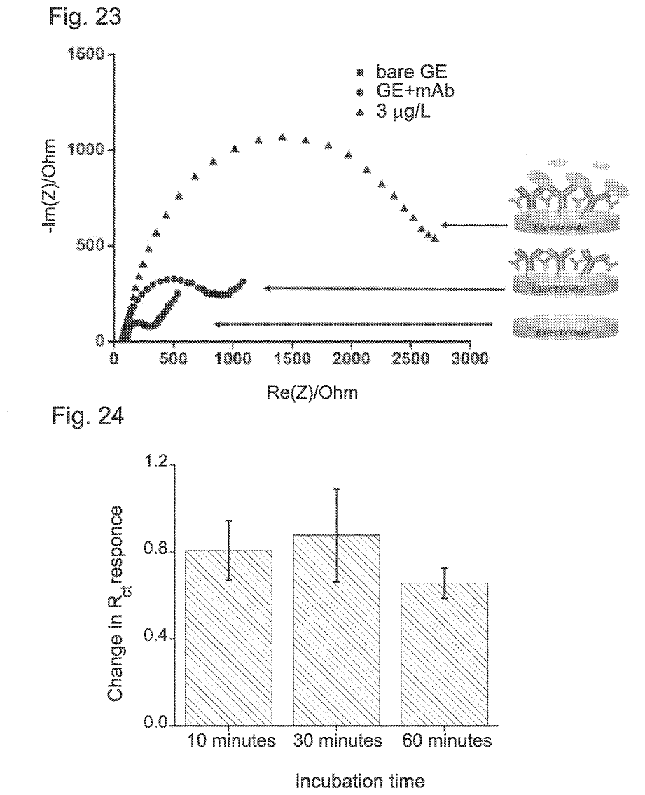

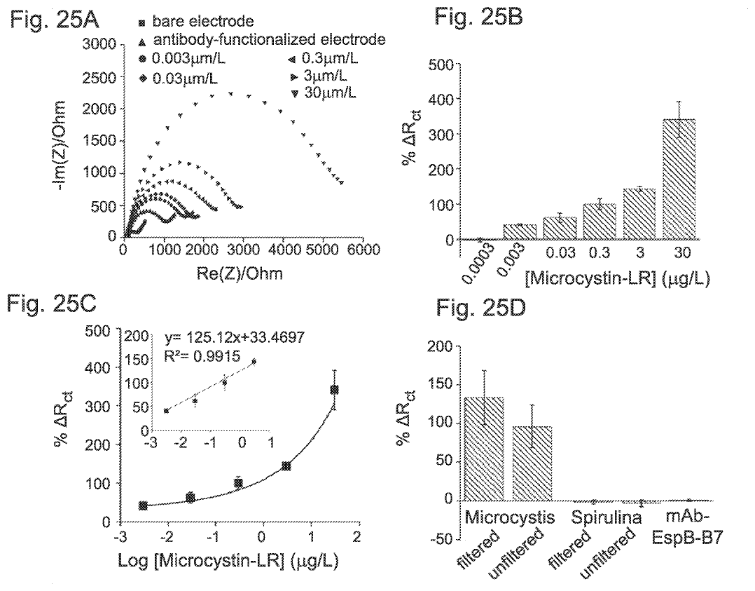

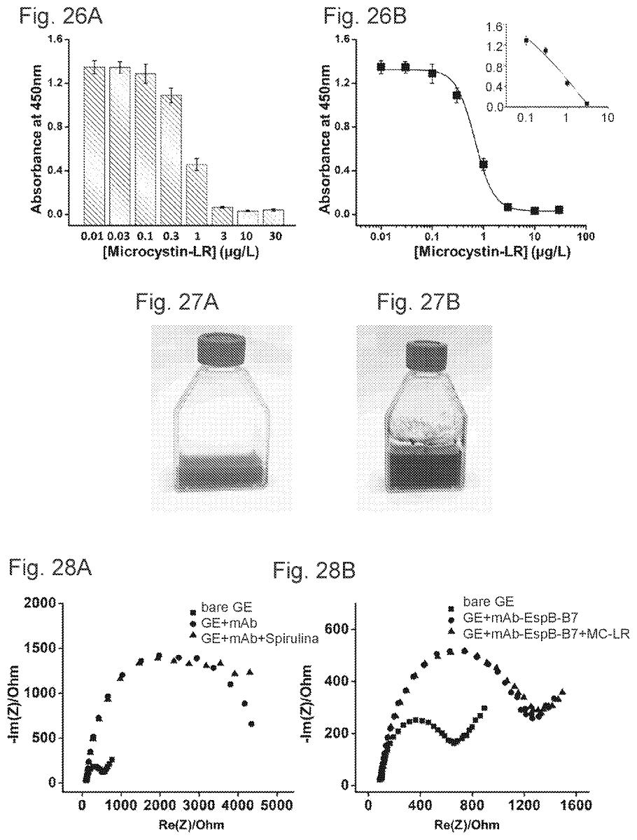

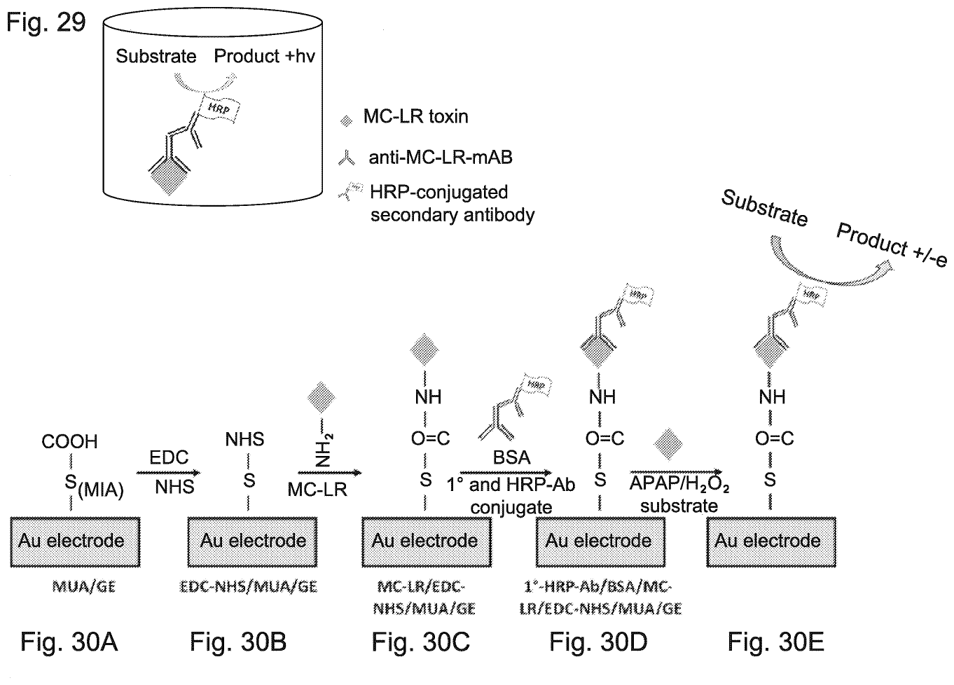

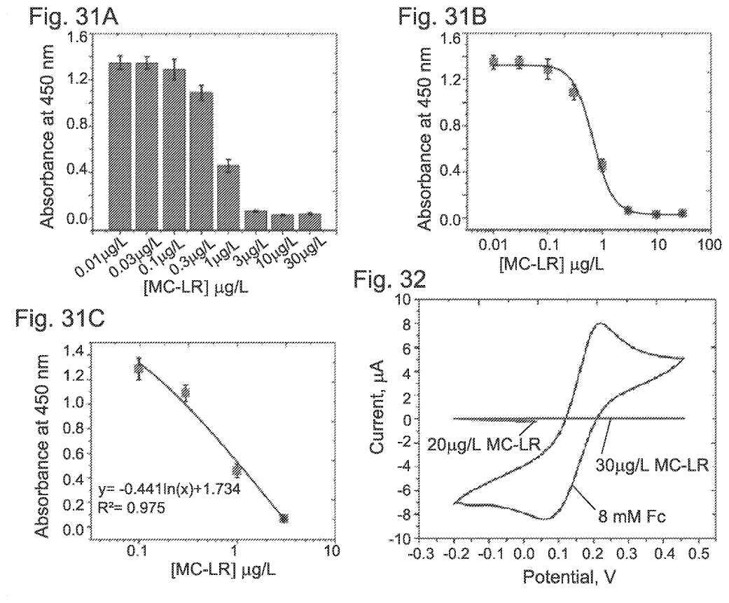

Next, the ability of mAb-EspB-B7 to bind EspB was evaluated under various conditions by ELISA. It was found that incubation of mAb-EspB-B7 in human serum did not compromise the ability of the antibody to bind EspB compared to its binding in a 3% milk solution (FIG. 3A). Examination of mAb-EspB-B7 binding under different pH conditions demonstrated that the binding was essentially not altered under a wide range of pH values (5.6-7.4), with the exception of pH 4.6, at which (as expected) there was a reduction in binding capacity (FIG. 3B). Interestingly, testing mAb-EspB-B7 across a wide range of NaCl concentrations demonstrated that only increased salt concentrations (>250 mM) affected the binding capacity of the antibody (FIG. 3C). Finally, to assess the thermal stability of mAb-EspB-B7, the melting temperature of the antibody was determined, both alone and in complex with purified EspB, by using nanoDSF. The melting temperatures of 75.4° C. and 82° C. for mAb-EspB-B7 alone and in complex with EspB, respectively (FIG. 4), indicated high mAb stability.

### Example 4

Non-Interference of mAb-EspB-B7 with the EspB-EspD Interaction

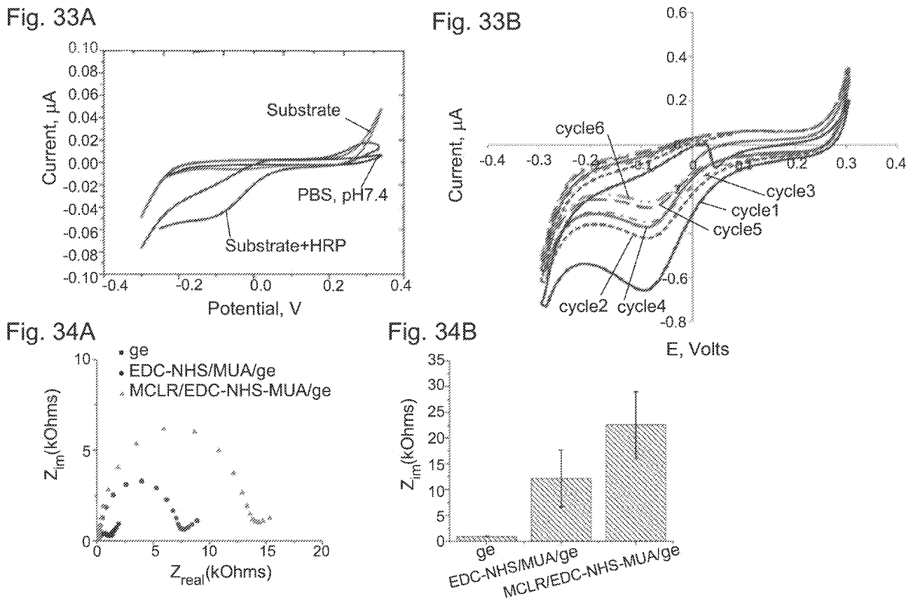

The EspB protein is found in a complex with another T3SS protein, called EspD, within the assembled T3SS. To confirm that the mAb-EspB-B7 epitope is exposed following EspB-EspD interaction, the ability of EspB to co-elute with EspD in the absence or the presence of mAb-EspB-B7 (100 or 200 nM), was evaluated. As observed in FIG. 5, the presence of mAb-EspB-B7 did not affect the co-elution of EspB with EspD, suggesting that mAb-EspB-B7 does not interfere with the EspB-EspD interaction. Low non-specific binding of EspB to the Ni-NTA beads was observed in the negative control (a sample that did not express EspD-³⁵His).

### Example 5

mAb-EspB-B7 Epitope Mapping

To identify the exact epitope of mAb-EspB-B7 within the EspB protein, a peptide array of 78 cyclic peptides that covers the full sequence of EspB (321 residues long), was designed. Each peptide was 15 residues long, with an overlap of 11 residues between the peptides. Recombinant EspB (full-length) served as a positive control, while BSA served as a negative control. Incubation of mAb-EspB-B7 with the peptide array revealed that mAb-EspB-B7 bound mostly to two cyclic peptides within the array, namely, to peptides #49 (positions 193-207) and #50 (positions 197-211), which have the sequences TSAQKASQVAEEAAD (SEQ ID NO. 33) and KASQVAEEAADAAQE (SEQ ID NO. 35) of the EspB protein, respectively (FIG. 6A). To confirm that this epitope is indeed recognized by mAb-EspB-B7, the following peptides were synthesized: peptide #49; peptide #50; a peptide that comprises the combined sequences of peptides #49 and #50 (TSAQKASQVAEEAADAAQE) (SEQ ID NO. 37); peptide #78 (SEQ ID NO. 38), which was not detected by the mAb-EspB-B7 and was therefore suitable as a negative control; and two peptides with scrambled sequences of peptides #49 and #50. Competitive ELISA between full-length EspB and the cyclic peptides revealed that pre-incubation of peptides #49, #50 or #49+#50 (1 μg/ml) (SEQ ID NOs. 33, 35, 37, respectively), with mAb-EspB-B7 completely abolished the ability of the antibody to bind full-length EspB (FIG. 6B-6D). To determine whether the competitive effect is derived directly from the binding of mAb-EspB-B7 to the peptides, the ability of mAb-EspB-B7 to recognize and bind these peptides was assessed. As observed in FIG. 7, mAb-EspB-B7 can bind peptides #49, #50 (SEQ ID NOs. 33, 35) and the combined peptide (#49+#50) (SEQ ID NOs. 37), while no binding was detected for the scrambled peptides or for peptide #78 (FIG. 7A-7C). These results confirm that the main mAb-EspB-B7 epitope is the KASQVAEEAAD sequence of the EspB protein (peptide sequences are presented in FIG. 7D) (SEQ ID NO. 39).

### Example 6

mAB-EspB-B7 Specificity Towards EPEC EspB Homologs

To assess the specificity of mAb-EspB-B7 toward EspB homologs in other bacterial pathogens and its potential to be used for detection of bacteria related to other infectious diseases, bacterial cultures grown under T3SS-inducing conditions were centrifuged, and supernatants and pellets were analyzed by SDS-PAGE and western blotting using mAb-EspB-B7. The following WT bacteria and T3SS-mutant strains were cultured: EPEC; enterohemorrhagic E. coli (EHEC), which causes a more severe disease than EPEC in humans; C. rodentium, an EPEC-related mouse pathogen; and Salmonella enterica serovar Typhimurium, which utilizes two T3SSs for virulence. The strongest signal was observed for the WT EPEC supernatant; a significant, but less strong, signal was also detected for C. rodentium, and an even less strong signal, for EHEC (FIG. 8A). However, no signals were detected in the supernatants of Salmonella or any of the T3SS mutant strains. Sequence alignment of EPEC and C. rodentium EspB proteins revealed high conservation between the proteins and full conservation of the mAb-EspB-B7 epitope sequence. Sequence alignment between EPEC and EHEC EspB proteins revealed 80% similarity at the mAb-EspB-B7 epitope region (FIG. 8B).

### Example 7

Effect of mAb-EspB-B7 on Bacterial-Host Cell Interaction

To examine the ability of mAb-EspB-B7 to directly interfere with the bacterial infection of host cells, the translocation activity of WT EPEC was examined in the presence or absence of mAb-EspB-B7. For that purpose, HeLa cells were infected with EPEC strains (WT and ΔescN) and the cleavage pattern of JNK was examined, a host protein that is cleaved by a translocated EPEC effector known as NleD (FIG. 9A). WT EPEC caused extensive degradation of JNK, relative to the uninfected sample and the samples infected with the ΔescN mutant strain (FIG. 9B). The inventors found that, HeLa cells infected by WT EPEC in the presence of a high concentration of mAb-EspB-B7 (400 nM) showed translocation activity similar to that of WT EPEC infection with no addition of antibody. These results suggest that while mAb-EspB-B7 was capable of binding EspB with high affinity and specificity, this binding did not interfere with the protein function and did not inhibit the T3SS translocation activity in an ex vivo model.

### Example 8

Development of mAb-EspB-B7-Based Biosensor and its Application in Detecting Purified EspB Protein and EspB-Presenting Bacteria

The potential of the developed mAb-EspB-B7 in diagnostic applications may be exploited by integrating it with an electrochemical biosensor. In particular, impedimetric immunosensors show great promise in rapidly detecting low concentrations of target antigens within a highly simplified testing setup [Randviir, et al. Analytical Methods 2013, 5 (5), 1098-1115; Siddiqui, S. et al. Biosens Bioelectron 2012, 35 (1), 284-290]. A general scheme of the disclosed device is shown in FIG. 11. FIG. 11 shows an electrochemical chip device providing an electrode arrangement within contact with a sample. The sample is provided by a sample collector and may be pushed into a measurement chamber using a syringe/plunger. The inventors sought to demonstrate this potential by constructing a miniature electrochemical biochip functionalized with mAb-EspB-B7, as illustrated in FIG. 12A working electrode of the electrode arrangement is pre-treated by biofunctionalization with mAb-EspB-B7. Impedance measurement between the electrodes provides data indicative of agents bound to the binding site. The impedance measurements may be represented by Nyquist plots that were fitted to an equivalent model circuit from which charge transfer resistance (R_{ct}) values were obtained. The EIS was recorded for the electrodes before and after antibody immobilization and these were compared with measurements taken after a 10 min incubation of purified EspB protein at varying concentrations. Using the Nyquist plot (FIG. 12B) the effect of mAb-EspB-B7 immobilization on the R_{ct} was observed. In a bare electrode, the resistance to charge transfer is small and impedance is dominated by mass transfer (diffusion of the electroactive species), the so-called Warburg impedance, which is evident in low frequencies [17]. The Warburg impedance is considerably decreased following mAb immobilization as mass transfer is no longer a significant factor. Instead, the contribution of R_{ct} to the impedance is now largely dominant, as an insulating layer of biomolecules is attached to the surface. Following a brief incubation of 250 μg/mL purified EspB solution, a significant increase in R_{ct} was observed, which is directly correlated with the bound antigen concentration further adding to the resistive component of the impedance. It was found that the addition of purified EspB protein affects the R_{ct} in a dose-dependent manner, enabling the distinction between different concentrations (FIG. 12C). In addition, incubation of a non-specific antigen (microcystin toxin) showed no effect on the measured R_{ct}. Similarly, no effect was observed from purified EspB protein directly incubated on a bare electrode (FIG. 10C), indicating that the change in R_{ct} reflects the specific binding of EspB to the mAB-EspB-B7 antibody. The relative change in R_{ct} exhibits an exponential dependence on concentration, as seen in FIG. 12D.

To examine the applicability of mAb-EspB-B7-based electrochemical bio-sensing in detecting whole bacteria harboring T3SS (and present EspB), WT EPEC and ΔespB mutant strains were grew under T3SS-inducing conditions and incubated bacterial samples on the biochip. As shown in FIG. 12E, higher R_{ct} values were consistently recorded for EPEC WT (mean R_{ct} change=1.22±0.09) compared to the ΔespB mutant strain (mean R_{ct} change=0.86±0.12). The minimal binding of the ΔespB strain was likely due to nonspecific adsorption. It should be noted that following centrifugation and lack of T3SS-inducing conditions, shedding of the complex is likely to occur, consistent with the western blot analysis shown in FIG. 2A. Nevertheless, the obtained signals were shown to be significantly higher (36%±15) in response to EPEC WT compared to the ΔespB strain (p=0.03). Overall, these results demonstrated the potential of the biosensor of the present disclosure, to detect EspB, both in its secreted form as well as an integral component of the assembled T3SS complex in the context of whole bacteria.

### Example 9

### Preparation and Characterization of the Impedimetric Immunosensors

Impedimetric immunosensors show great promise in rapidly detecting low concentrations of target antigens within a highly simplified testing setup. The inventors sought to demonstrate this diagnostic potential by developing a miniature electrochemical biochip, integrating it with a monoclonal antibody (mAb) targeted against the microcystin MC-LR, and applying the developed immunosensor in the rapid detection of low MC-LR concentrations. A schematic illustration of the developed biochip is presented in FIG. 17A.

Multiple electrochemical cells were fabricated and functionalized to enable real-time detection of MC-LR from contaminated water samples. Electrochemical Impedance Spectroscopy (EIS) measurements yielded Nyquist plots that were fitted to an equivalent circuit from which charge transfer resistance (R_{ct}) values were obtained, as shown in FIG. 17B.

The developed biochip, comprising a three-electrode electrochemical cell, was fabricated by a robust process optimized for wafer-scale manufacturing with a yield of ˜80%, as shown in Supplementary FIG. 17C, and assembled into a custom-designed, Polytetrafluoroethylene (PTFE) measurement platform, shown in FIG. 18. An on-chip Ag/AgCl quasi-reference electrode was also electroplated to enable miniaturization and consequently, high-throughput measurements (shown in FIG. 17D). Chips were characterized electrochemically prior to experiments, as shown in FIGS. 19 and 20.

Multiple biochips were simultaneously interrogated by the measurement platform, which also provides electrical contacts for the biochips and connects to a potentiostat device. The designed platform further enables hydrodynamic measurements.

Impedimetric immunosensors are based on immobilized antibodies to detect antigens using EIS on a solid-state electrode. The immobilization strategy of antibodies is of critical significance in the development because it determines the orientation of the antibody on the electrode's surface [N. G. Welch, et al., Biointerphases. 12 (2017)]. The immobilization approach used by the present disclosure is based on the direct covalent attachment of thiolated antibodies to a gold electrode surface. The thiolation reaction was optimized to obtain an average of ˜6 —SH group per antibody by tuning the ratio of reagent to antibody. This fine-tuning enables control of the level of thiolation and ensures that antibody molecules are introduced with a sufficient number of thiols allowing their immobilization. Estimation of introduced sulfhydryl groups was performed by Ellman assay that is used to quantify the number or concentration of thiol groups in a sample. Further details including the thiolation mechanism and Ellman assay are described in the experimental procedure section and in FIG. 21. Gold surfaces can be readily reacted with the sulfur head of thiolated molecules enabling the immobilization of biorecognition molecules [M. P. Chatrathi, et al., Biosens. Bioelectron. 22 (2007) 2932-2938]. An assessment of the immobilization efficiency was carried out by fluorescence microscopy analysis, using a fluorescently (Cy3)-labeled thiolated antibody compared with non-thiolated antibody. Fluorescence microscopy images shown in FIG. 22A confirm the immobilization of antibodies to the gold electrode. Electrode surface characterization by AFM, as shown in FIG. 22B, provides further indication for the immobilization of antibodies.

Finally, this direct approach to electrode functionalization is advantageous compared to well-established self-assembled monolayer (SAM) generation methods since it involves a straightforward preparation and avoids complete electrode passivation often attained with SAM. Furthermore, this functionalization procedure can be readily scaled up as it is compatible with microarray printing technology.

To measure the effect of immobilized antibodies on the R_{ct}, impedance spectra were recorded and analyzed before and after antibody immobilization, and these were compared with measurements taken after 30 minutes of incubation with MC-LR, as shown in FIG. 23. It should be noted, that a 10 min incubation was found to significantly affect the recorded impedimetric signal, as shown in FIG. 24. Using the Nyquist plots, we observed the effect of antibody immobilization on the R_{ct}. In a bare electrode, the R_{ct} is small and impedance is dominated by the diffusion of the electroactive species, the so-called Warburg impedance, which is evident in low frequencies [A. Lasia, Electrochemical Impedance Spectroscopy and its Applications—Andrzej Lasia—Google Books, 2014]. Following antibody immobilization, the Warburg impedance is no longer a significant factor. Instead, the contribution of R_{ct} to the impedance becomes largely dominant, as an insulating layer of biomolecules is attached to the surface. Incubation with a solution containing 3 μg/L MC-LR resulted in a further increase in R_{ct} as the bound toxin further adds to the resistive component of the impedance.

### Example 10

### Evaluating the Sensitivity and the Dynamic Range of the Device

The sensitivity and dynamic range of the device were measured by briefly immersing biochips in MC-LR solutions with concentrations spanning six orders of magnitude, from 0.0003 μg/L to 30 μg/L, as shown in the Nyquist plots of FIG. 25A. The addition of MC-LR was found to affect R_{ct} in a dose-dependent manner, clearly seen in FIG. 25B, with a positive correlation observed between MC-LR concentration and R_{ct} response. The calibration curve of the R_{ct} response exhibited an exponential dependence (R²=0.9812) (FIG. 25C). Nonlinear calibration curves have been previously reported in impedimetric biosensors[47,48]. The inset in FIG. 25C shows a linear response (R²=0.9915) to the log of MC-LR concentrations ranging between 0.003 μg/L to 3 μg/L, with 0.003 μg/L yielding a mean R_{ct} increase of 40.8% (±3.4) while 3 μg/L affected a mean R_{ct} increase of 143.3% (±10.1).

When developing new sensors for diagnostic assays it is paramount to compare the results with these obtained from assays that are considered the gold standard in the field. In the case of microcystin detection, ELISA (enzyme-linked immunosorbent assay) is routinely employed by different water suppliers to ensure safe drinking water. In an orthogonal study, the inventors have developed an indirect competitive ELISA, as shown in FIG. 26, using the same MC-LR antigen and mAb that were used in the impedimetric immunosensor. The detection limit was found to be higher by nearly two orders of magnitude compared to that obtained by the biosensor (see FIG. 26A, 26B) and the dynamic range (see FIG. 26B, inset) was significantly narrower, indicating the improved sensitivity of electrochemical biosensing compared with ELISA.

### Example 11

### Assessing the Applicability of the Biosensor

The applicability of the immunosensor was further demonstrated by detecting MC-LR in bacterial suspensions of Microcystis aeruginosa PPC 7806 and Spirulina sp., used as models for contaminated water. These samples represent highly complex matrices containing various aquatic bacteria, biomolecules and cell debris, as evident by the images shown in FIG. 27. Both raw and filtered Microcystis aeruginosa PPC 7806 samples (2.08×10⁷ cells/mL) demonstrated an increased impedimetric response, as shown in FIG. 25D. Higher R_{ct} values were consistently recorded for filtered samples (mean R_{ct} change=1.33±0.77), compared to unfiltered samples (mean R_{ct} change=0.85±0.46), both relative to the antibody-modified electrode (FIG. 25D). Incubation with the non-CT-producing Spirulina suspension (4.9×10⁷ cells/mL), used as a negative control, did not affect the recorded impedance, as shown in FIG. 25D. In addition, no response was observed when MC-LR was incubated on electrodes functionalized with a non-specific antibody, mAb-EspB-B7 (that targets the virulent EspB protein in pathogenic E. coli [Y. Hillman, et al., Anal. Chem. 93 (2021)]). The Nyquist plots of these control measurements, shown in FIG. 28, clearly demonstrate the specificity towards MC-LR, lending further support to the feasibility of the detection method of the present disclosure.

It should be noted that the specificity towards MC-LR, as reported here, should more accurately be regarded as specificity towards the prominent MC variants. all commercial antibodies developed against MC-LR demonstrate variable cross-reactivities with other MC variants, particularly MC-RR and MC-YR, as they target either the 4-arginine in these isomers, or the Adda group common to all MCs [M. G. Weller, et al., Anal. Sci. 17 (2001) 1445-1448; A. Zeck, et al., Analyst. 126 (2001); J. Sheng, et al., Front. Environ. Sci. Eng. China. 1 (2007) 329-333; J. W. Sheng, et al., Anal. Chim. Acta. 603 (2007) 111-118; T. Vinogradova, et al., Talanta. 84 (2011) 638-643; R. A. Halvorson, Raman Spectroscopy, (2011) 9273-9280]. Consequently, since they cannot distinguish between these three common isomers, immunodiagnostic assays typically report results as ‘MC-LR equivalents’, MC-LR being the most toxic and most commonly encountered form [F. Gurbuz, et al., Turkey, Sci. Total Environ. 407 (2009) 4038-4046; X. Su, et al., Ecol. Indic. 89 (2018) 445-454; R. P. Rastogi, et al., current overview, (2014) 215-249]. This is also the accepted measure for departments of environmental protection and water providers around the world that routinely use commercial ELISA kits to monitor MC-LR concentrations. Thus, the specificity of this new generation of immunosensors is expected to be similar to available technologies.

### Example 12

### MC-LR Biosensor for Electrochemical Indirect Competitive ELISA

The inventors next developed a n substrate-mediated amperometric immunoassay for detecting the MC-LR toxin. The Electrochemical (EC) immunodetection of Microcystin-LR (MC-LR) is performed by an EC indirect competitive (ic) ELISA. Substrate-mediated amperometric detection of MC-LR is performed whereby the level of surface-bound enzyme immunoconjugate following catalysis of an electroactive substrate is evaluated by a chronoamperometric detection using various standard MC-LR concentrations and raw cyanobacteria culture samples. The design and fabrication of a biosensing system that enables amperometric immunodetection of MC-LR is presented in the experimental procedure section. This system has been further characterized as detailed herein.

### MC-LR Detection Based on Indirect Competitive ELISA

The ic-ELISA, measured in eight repeats, and a standard curve are shown in FIGS. 31A and 31B. The primary antibody MC10E7 dilution was 1:3,000 (333 ng/ml); secondary antibody dilution was 1:4,000 (200 ng/ml). The vertical error bars represent the standard deviation (n=8). The coefficient of variation was less than 10%. The detection limit of MC-LR was attained at 0.03 μg/L with an upper limit of 3 μg/L, as shown in FIG. 31B. The quantitative detection range was from 0.1 μg/L to 3 μg/L, showing a linear fit, as shown in FIG. 31C.

### EC Characterization of MC-LR

The EC properties of MC-LR were studied by CV at a potential range of 0.2 V to 0.5V for MC-LR concentrations: 20 μg/L and 30 μg/L. The voltammograms are shown in FIG. 32 reveal that MC-LR is intrinsically not electroactive at the measured potential range.

### EC Analysis of Enzyme and Substrate

The reaction of HRP with acetaminophen (APAP) is characterized electrochemically. The indirect measurement correlate the signal from the electro-reduction of APAP to the concentration of the antigen. A CV was carried out to determine the potential for the electro-reduction of NAPQI and to verify that in this potential, APAP and H₂O₂ are both electrochemically inert. At the applied potential window (−0.3 V to +0.3 V) no APAP oxidation. APAP/H₂O₂ is not electroactive in these low potentials (FIG. 33). The EC characterization of NAPQI was performed by first generating it via the reaction of HRP with APAP (in the presence of H₂O₂). The data presented in FIG. 33 show that a distinct cathodic current peak occurs at a potential of E=−100 mV, which reflects NAPQI's EC reduction. This potential value is high enough to function in the diffusion-controlled region and is still small enough to ensure minimal interference in solution and background signals from possible electro-active species. Under these conditions, these scans simulate an irreversible process, which is characterized by diminishing peak currents, as observed above, following the equation: iₚ=(2.99×10⁵)α^{1/2}AC₀D₀^{1/2}υ^{1/2} where A is in cm² (electrode area), C⁰ is in molar (bulk concentration), D⁰ is in cm²/s (diffusion coefficient), υ is in V/s (scan rate) and α is the transfer coefficient. The peak current is still proportional to the bulk concentrations but is expected to be lower in height (depending upon the value of a), comparing to the behaviour of reversible systems according to the Randles-Sevcik equation. This behaviour was observed in the first three cycles and the next three cycles.

### EC of MC-LR and its Immobilization Using Self Assembled Monolayer (SAM)

FIG. 34A shows the results of Faradic impedance spectroscopy on the bare GE, the activated MUA-modified gold electrode with EDC/NHS, and the MC-LR toxin immobilization on the activated surface, in the presence of the redox couple Fe(CN)₆^{3-/4-} measured at the formal potential of the redox couple. The formal potential value of 100 mV was determined from the CV curve of the bare gold working electrode. The impedance plot of the bare gold electrode is characterized by a small semi-circle at a high-frequency domain, corresponding to an interfacial charge transfer mechanism. The semicircle represents a parallel combination of the charge-transfer resistance, R_{ct}, and the double-layer capacitance, C_{DL}, whereas the linear response is associated with mass transport processes. The impedance spectra of the bare gold electrode were fitted with the Randles equivalent circuit to determine the electrical properties of the bare GE. The circuit includes the background solution resistance, Rₛ, the resistance to charge transfer, R_{ct}, and the Warburg impedance, Z_{W}, which results from the diffusion of the redox couple from the bulk of the solution to the electrode interface.

After preconditioning the bare GE with 1 mM of MUA solution for 16 hrs. at room temperature, and activating the COOH end groups with EDC/NHS chemistry, the diameter of the semi-circle corresponding to the charge transfer resistance is significantly increased. This is attributed to the physical barrier of the assembled layer that prevents the access of the redox couple. Following immobilization with the MC-LR toxin, a further increase in the R_{c}t was observed because of an additional barrier created towards the access of the redox probe to the GE surface (FIG. 34B).

1-48. (canceled)

49. A biosensor chip system usable for identifying and/or quantifying and/or monitoring at least one target in a sample; the system comprises at least one of: at least one first and at least one second chip devices; wherein:
said first chip device comprises a first plurality of electrodes connectable to at least one electronic device; wherein at least one of said electrodes is a working electrode, said working electrode is connected directly or indirectly to at least one target binding site and/or moiety, wherein said target binding site and/or moiety specifically binds said at least one target or any component thereof, and wherein said first plurality of electrodes is configured for electrochemical impedance spectroscopy (EIS) analysis of said sample; and
said second chip device comprises:
a second plurality of electrodes connectable to at least one electronic device; wherein at least one of said electrodes is a working electrode, said working electrode is connected directly or indirectly to said target or any component thereof, and wherein said second plurality of electrodes is configured for electrochemical voltammetry or amperometry analysis of said sample.

50. The biosensor chip system according to claim 49, further comprising a packaging assembly configured to sealably enclose said electrodes portion of the substrate and define at least one measurement chamber encompassing said electrodes, optionally, wherein the biosensor chip system comprising said first and second chip devices, and wherein respective pluralities of electrodes of said first and second chip devices are positioned in respective first and second separated measurement chambers.

51. The biosensor chip system according to claim 50, further comprising at least one inlet for introducing said sample into said measurement chamber; and
at least one inlet filter for selectively passing said sample from said inlet into said measurement chamber, optionally, the biosensor chip system comprising an outlet formed in the packaging assembly and at least one outlet filter for selectively passing sample material from the measurement chamber to said outlet.

52. The biosensor chip system according to claim 50, wherein at least one of:
(a) the packaging assembly comprises a base portion configured to receive the electrodes portion of the substrate, and a cover portion having an open cavity and configured to sealably attach to said base portion over said electrodes portion of the substrate and define the measurement chamber by its open cavity;
(b) wherein the first and second plurality of electrodes of at least one of said first and second chip devices comprises at least one working electrode, at least one counter electrode configured to vary electrical potential and enable current transmission into said measurement chamber, and at least one reference electrode for measuring electrical voltage between said at least one working electrode and said at least one reference electrode; and
(c) said at least one electronic device comprises one or more potentiostat circuitries connected at said one of first and second chip devices.

53. The biosensor chip system according to claim 49, wherein said at least one electronic device comprises a plurality of potentiostat circuitries, said system comprising a plurality of measurement chambers comprising at least one first measurement chamber associated with said first chip device and at least one second measurement chamber associated with said second chip device, each of said measurement chambers comprises at least three of the plurality of electrodes defining a working electrode, a reference electrode, and a counter electrode, and is associated with respective potentiostat circuitries electrically connected to the at least three electrodes of its respective measurement chamber, optionally, wherein said plurality of potentiostat circuitries comprises at least one first potentiostat circuitry associated with electrodes of said first chip device and configured for operating electrochemical impedance spectroscopy (EIS), and at least one second potentiostat circuitry associated with electrodes of said second chip device and configured for operating at least one of voltammetry and amperometry measurement.

54. The biosensor chip system according to claim 49, wherein said system comprises a plurality of one or more first chip devices and one or more second chip devices located in a plurality of separated measurement chambers, the respective pluralities of electrodes comprises a plurality of working electrodes, reference electrode, and counter electrodes, and wherein the device comprises a potentiostat circuitry and a multiplexer device configured to selective transfer signals between the respective pluralities of electrode to said potentiostat circuitry.

55. The biosensor chip system according to claim 49, wherein said target is at least one small molecule compound, optionally, wherein at least one of:
(a) said small molecule compound is at least one toxin;
(b) said toxin is at least one cyanotoxin;
(c) wherein said cyanotoxin is at least one of: at least one cyclic peptide, at least one alkaloid and at least one lipopolysaccharide, or any combinations thereof; and
(d) said cyanotoxin is at least one cyclic peptide, said cyclic peptide is at least one microcystin (MC), and at least one nodularin (NOD).

56. The biosensor chip system according to claim 55, wherein small molecule compound is at least one toxin; and wherein at least one of:
(a) said toxin is at least one microcystin, said microcystin is at least one of Microcystin-leucine-arginine (MC-LR), Microcystin-arginine-arginine (MC-RR), Microcystin-tyrosine-arginine (MC-YR), and Microcystin-leucine-alanine (MC-LA), and any combination, derivatives and variants thereof; and
(b) wherein said microcystin is Microcystin-LR (MC-LR), or any derivatives and variants thereof.

57. The biosensor chip system according to claim 49, wherein at least one of:
(i) said at least one working electrode of said first chip device is connected directly or indirectly to at least one antibody that specifically binds said at least one cyanotoxin; and
(ii) said at least one working electrode of said second chip device is connected directly or indirectly to said at least one cyanotoxin.

58. The biosensor chip system according to claim 49, wherein said sample is an environmental sample or a biological sample.

59. A kit comprising:
(a) at least one biosensor chip system as defined in claim 49, usable for identifying and/or quantifying and/or monitoring at least one target in a sample; the system comprises at least one of: at least one first and at least one second chip devices; wherein:
said first chip device comprises a first plurality of electrodes connectable to at least one electronic device; wherein at least one of said electrodes is a working electrode, said working electrode is connected directly or indirectly to at least one target binding site and/or moiety, wherein said target binding site and/or moiety specifically binds said at least one target or any component thereof, and wherein said plurality of electrodes is configured for electrochemical impedance spectroscopy (EIS) analysis of said sample; and
said second chip device comprises:
a second plurality of electrodes connectable to at least one electronic device; wherein at least one of said electrodes is a working electrode, said working electrode is connected directly or indirectly to said target or any component thereof, and wherein said plurality of electrodes is configured for electrochemical voltammetry or amperometry analysis of said sample;
optionally, said kit comprises at least one of:
(b) at least one control sample and/or control standard value;
(c) instructions for use.

60. A method for identifying and/or quantifying and/or monitoring at least one target in a sample, the method comprising at least one of:
(a) performing an electrochemical impedance spectroscopy (EIS) analysis of said sample, comprising:
(i) contacting with said sample a first plurality of electrodes comprising at least one working electrode and at least one reference electrode, or any first chip device or system comprising the same, wherein said at least one working electrode is connected directly or indirectly to at least one target binding site and/or moiety;
(ii) applying voltage signal between said at said least one working electrode and said at least one reference electrode, and determining electrical current between said electrodes in response to said voltage signals for a selected number of one or more signal frequencies; and
(iii) determining relations between electrical current response and voltage signal for said one or more signal frequencies; and determining electrical impedance between the at least one working electrode and the at least one counter electrode;
wherein impedance variation being indicative of presence and/or quantity of said at least one target in said sample; and/or
(b) performing an electrochemical voltammetry or amperometry analysis of said sample, comprising:
(i) contacting with said sample a second plurality of electrodes comprising at least one working electrode and at least one reference electrode, or any second chip device or system comprising the same, wherein said at least one working electrode is connected directly or indirectly to said at least one target or any component thereof; and wherein said sample further comprises at least one first binding molecule specific for said at least one target, and at least one second binding molecule specific for said first binding molecule, wherein said second binding molecule comprises at least one labeling moiety that comprises and/or produces at least one electroactive product;
(ii) applying voltage signal between said at least one working electrode and at least one reference electrode and determining electrical current through said at least one working electrode in response to varying voltage signal; and
(iii) determining peak current value, said peak current value is inversely indicative of presence and/or quantity of said at least one target.

61. The method according to claim 60, wherein said sample is subjected to an electrochemical impedance spectroscopy (EIS) analysis, and wherein said method further comprising processing electrical impedance determined based on one or more voltage signal frequencies for determining charge transfer electrical resistance between the at least one working electrode and the at least one counter electrode, and determining presence of said at least one target in said sample whenever said charge transfer electrical resistance is greater than a predetermined threshold value, optionally, wherein determining the charge transfer electrical resistance comprises determining an electrical circuit model representing charge transfer between the electrodes, said electrical circuit may comprise capacitance model connected in parallel to inductance model and charge transfer electrical resistance model, thereby allowing to determine charge transfer electrical resistance in accordance with total impedance of the circuit.

62. The method according to claim 60, wherein said sample is subjected to an electrochemical voltammetry or amperometry analysis, and wherein said method further comprises applying said peak current value determined for said sample on a predetermine standard curve for determining concentration of said at least one target in said sample.

63. The method according to claim 62, wherein at least one of:
(a) said at least one labeling moiety of said at least one second binding molecule, produces at least one electroactive product;
(b) said labeling moiety comprises at least one enzyme that catalyzes the conversion of at least one substrate into at least one electroactive product;
(c) wherein said enzyme is at least one of horseradish peroxidase (HRP), and alkaline phosphatase (ALP), optionally, at least one of:
(i) said enzyme is HRP that catalyzes the oxidation of at least one substrate, wherein at least one of said substrate is acetaminophen; and
(ii) wherein said method comprises the step of providing said sample with an effective amount of acetaminophen.

64. The method according to claim 62, wherein at least one of:
(a) said at least one labeling moiety of said at least one second binding molecule comprises at least one electroactive product, preferably, said labeling moiety is at least one Ferrocene molecule; and
(b) said at least one first binding molecule is at least one primary antibody specific for said at least one target, and wherein said at least one second binding molecule, is at least one secondary antibody specific for said primary antibody.

65. The method according to claim 60, wherein said target is at least one small molecule, optionally, said small molecule is at least one toxin, said toxin is at least one cyanotoxin, said cyanotoxin is at least one of: at least one cyclic peptide, at least one alkaloid and at least one lipopolysaccharide, or any combinations thereof, wherein said cyanotoxin is at least one cyclic peptide, said cyclic peptide is at least one microcystin (MC), and at least one nodularin (NOD), said microcystin is at least one of Microcystin-leucine-arginine (MC-LR), Microcystin-arginine-arginine (MC-RR), Microcystin-tyrosine-arginine (MC-YR), and Microcystin-leucine-alanine (MC-LA), and any combination, derivatives and variants thereof.

66. The method according to claim 60, wherein at least one of:
(a) said at least one working electrode of said first chip device is connected directly or indirectly to at least one antibody that specifically binds said at least one cyanotoxin; and/or said at least one working electrode of said second chip device is connected directly or indirectly to said at least one cyanotoxin; and
(b) said sample is an environmental sample or a biological sample, optionally, said environmental sample comprises at least one sample obtained from natural or artificial water reservoir, reclaimed water, and wastewater treatment and sewage treatment.

67. A method of treating, preventing, ameliorating, reducing or delaying the onset of a disorder associated with exposure to at least one toxin in a subject in need thereof, the method comprising:
(a) classifying a subject as exposed to said toxin if the presence of said at least one toxin is determined in at least one biological sample of said subject, or in at least one environmental sample associated with said subject, wherein determination of the presence of said at least one toxin in said sample is performed by at least one of:
(I) performing an electrochemical impedance spectroscopy (EIS) analysis of said sample, comprising:
(i) contacting with said sample a first plurality of electrodes comprising at least one working electrode and at least one reference electrode, or any first chip device or system comprising the same, wherein said at least one working electrode is connected directly or indirectly to at least one target binding site and/or moiety;
(ii) applying voltage signal between said at said least one working electrode and said at least one reference electrode, and determining electrical current between said electrodes in response to said voltage signals for a selected number of one or more signal frequencies; and
(iii) determining relations between electrical current response and voltage signal for said one or more signal frequencies; and determining electrical impedance between the at least one working electrode and the at least one reference electrode;
wherein impedance variation being indicative of presence and/or quantity of said at least one target in said sample; and
(II) performing an electrochemical voltammetry or amperometry analysis of said sample, comprising:
(i) contacting with said sample a second plurality of electrodes comprising at least one working electrode and at least one reference electrode, or any second chip device or system comprising the same, wherein said at least one working electrode is connected directly or indirectly to said at least one target or any component thereof; and wherein said sample further comprises at least one first binding molecule specific for said at least one target, and at least one second binding molecule specific for said first binding molecule, wherein said second binding molecule comprises at least one labeling moiety that comprises and/or produces at least one electroactive product;
(ii) applying voltage signal between said a least one working electrode and at least one reference electrode and determining electrical current through said at least one working electrode in response to varying voltage signal; and
determining peak current value, said peak current value is inversely indicative of presence and/or quantity of said at least one target;
thereby classifying said subject as exposed to said toxin; and
(b) administering to a subject classified as an infected subject in step (a), a therapeutically effective amount of at least one anti-toxin agent and/or additional therapeutic agent, optionally, wherein said toxin is cyanotoxin, preferably, MC-LR, and wherein said disorder associated with exposure to said MC-LR is at least one of liver damage, renal failure and neoplastic disorders and optionally, wherein the determination of the presence of said at least one toxin in said sample is performed by the method as defined by claim 60.

68. A method for identifying and/or quantifying at least one cyanotoxin in a sample, the method comprising:
contacting said sample with at least one working electrode, at least one reference electrode, and at least one counter electrode, or any biosensor chip or kit comprising said electrodes, wherein said at least one working electrode is connected directly or indirectly to at least one cyanotoxin binding site and/or moiety;
measuring electrical voltages between said at least one working electrode and said at least one reference electrode in response to electric currents of different frequencies applied between said at least one working electrode and said at least one reference electrode;
determining electrical impedances based on the measured electrical voltage and the electric currents applied at the different frequencies;
determining a charge transfer electrical resistance based on the determined impedances; and
determining presence of said at least one cyanotoxin in said sample whenever said charge transfer electrical resistance is greater than a predetermined threshold value.
